(19) United States
(12) Patent Application Publication
(10) Pub. No.: US 2024/0285782 A1
(43) Pub. Date: Aug. 29, 2024

(54) METHODS FOR USING BIOADHESIVE AND STERIC INTERACTIONS OF COPOLYMERS WITH AT LEAST TWO MOIETIES TO MINIMIZE ADVERSE EFFECTS MEDIATED BY EXTERNAL INFLUENCES ON CELL, TISSUE, ORGAN SYSTEM, AND ORGANISM BIOLOGY

(71) Applicant: Calm Water Therapeutics LLC, Rochester, NY (US)

(72) Inventor: David M. Kleinman, Rochester, NY (US)

(21) Appl. No.: 18/654,815

(22) Filed: May 3, 2024

Related U.S. Application Data
(63) Continuation of application No. PCT/US22/79406, filed on Nov. 7, 2022.
(60) Provisional application No. 63/276,321, filed on Nov. 5, 2021.

(51) Int. Cl. A61K 47/59; A61K 9/00; A61K 38/12; A61K 47/60; A61P 27/02

(52) U.S. Cl. CPC A61K 47/595; A61K 9/0051; A61K 38/12; A61K 47/60; A61P 27/02

(57) ABSTRACT

Methods for using bioadhesive and stearic interactions specific to copolymers with at least two moieties, to minimize adverse effects mediated by external influences on cell, tissue, organ system, and organism biology. Multifunctional copolymers have bioadhesive properties driven by electrostatic and hydrophobic interactions and passivation though hydrophilic moieties. These copolymers are useful for reducing rates of viral infectivity in target cells, and in reducing host morbidity and can be coforulated. These copolymers are useful for reducing ADC related, oncology therapy related, preservative related, systemic-pharmaceutical-therapy related, and/or topical ophthalmic active-pharmaceutical-ingredient related toxicity which can be manifest as a corneal epithelial toxicity. Methods for enhancing efficacy through coformulations with mucopenetration enhancing technologies and in coformulations with other actives that inhibit macropinocytosis allow for increasing efficacy in patient treatment approaches described herein. Coformulations with other actives ingredient will have therapeutic utility and prove beneficial in various settings. Formulations of these multifunctional copolymers for topical ophthalmic use are safe and well tolerated. Epithelial cells and the ocular surface including limbal stem cell and precursor corneal epithelial cells are treated with these copolymers to confer utility and patient benefit. Corneal nerves are similarly protected and corneal nerve cell damage is mitigated. Contact lenses provide an alternative method for delivering protective copolymers to the eye in these settings.

### PRIORITY

This application is a continuation of International Patent Application No. PCT/US2022/079406, filed Nov. 7, 2022, which claims the benefit of U.S. provisional application No. 63/276,321, filed Nov. 5, 2021, the contents of which are incorporated herein in their entireties.

### SEQUENCE LISTING

The instant application contains a Sequence Listing that is being submitted via Patent Center and is hereby incorporated by reference in its entirety. Said ASCII copy, created on Nov. 7, 2022, is named 3905-11302-seq-listing.xml and is 4 kb in size.

### FIELD OF THE INVENTION

The present invention provides a method for addressing serious problems currently encountered in the biomedical field by utilizing bioadhesive and passivation copolymers (containing a charged or hydrophobic moiety and a passivation hydrophilic moiety) to improve cellular, tissue, organ and mammalian health. Specifically, the field of this invention relates to the prevention, attenuation, reduction, or treatment of viral infections. And specifically, the field of this invention relates to the prevention, attenuation, reduction, or treatment of drug related toxicities associated with antibody-drug conjugates (ADCs) and their toxic payloads. “ADC” will be used frequently herein to represent an antibody-drug conjugate therapeutic. Enclosed herein are methods to improve the delivery of bioadhesive and passivation copolymers to at risk tissues (through for example, mucopenetration enhancement), extended-release approaches using contact lenses, and coformulations with other active agents that may help confer benefits to such cells, tissues, organs and mammals that are at risk from adverse events associated with drug products and at risk of viral infection.

ADCs are complex molecules composed of an antibody linked to a biologically active cytotoxic (anticancer) payload or drug. Antibody-drug conjugates may be types of bioconjugates and immunoconjugates. “ADC” can refer to a host of different antibody-drug conjugates herein. ADCs combine targeting capabilities inherent to monoclonal antibodies with the cancer-fighting ability of cytotoxic agents. They are often designed to discriminate between healthy and diseased cells or tissue.

The viral infection specifically of focus here is SARS-COV-2 infectivity. However, the key development is that the effect is useful for new viruses such that stearic and electrostatic interactions can inhibit the infectivity of a novel virus into a cell, tissue or organism for which host immunity has not yet developed or for which targeted antibody or antiviral therapy are not yet available. In other words, this effect is broad, effective and nonspecific. New viruses are amenable to treatment with this approach.

The ADC toxicity amelioration method is often related to off target entry of ADCs into non neoplastic cells and the adverse events associated with inhibiting cellular processes mediated by the ADC payload. At times, however, the nonneoplastic cells may express a target for the ADC allowing for its entry and interaction with these cells as well. Neoplastic cells are cancer cells. The ADCs are used to treat cancer or oncologic disease of one or many organ systems or cell types, including but not limited to renal cell cancers, leukemias, lymphomas, myelomas, lung cancer, prostate cancer, uterine or cervical cancer, breast cancer, bladder cancer, colon cancer, esophageal cancer, liver cancer, Hodgkin's disease, ovarian cancer, pancreatic cancer, rectal cancer, skin cancer, small bowel cancer, solid tumors, stomach cancer, white blood cell cancers, urethral cancer, mesenteric lymphadenitis. Any or all cancers here named and those unnamed can be combined with any claim for a specific invention with an ADC targeting such a cancerous cell or tissue.

Specifically, macropinocytosis mediated toxicity is inhibited, reduced, and limited via the steric and electrostatic interferences that take effect at the molecule-cellular-tissue (inclusive or isolated) level where the surface of a cell or tissue interacts with its local microenvironment. Cells include, but are not limited to limbal stem cells, transient amplifying cells, transient amplifying cells daughter cells, basal epithelial cells, wing cells, and corneal epithelial cells and differentiated corneal epithelial cells.

Importantly, other active pharmaceutical ingredients and excipients in topical ophthalmic drug products (and in some systemic formulations) can cause ocular irritation, discomfort, limit the use of a drug product, or cause corneal injury as manifested on slit lamp exam with or without fluorescein staining, and protection can be afforded in these settings by inventions disclosed herein.

Types of ocular toxicity that are iatrogenic, or medical therapy induced, are well known. For example, ocular preservatives can lead to ocular adverse events. Furthermore, some excipients can as well (higher concentrations of poloxamers, for example), and antiviral and antibiotic agents can have a direct toxicity on the corneal epithelial cells as another example.

Multifunctional graft copolymers have been identified as explained herein as a method to ameliorate, mitigate, reduce in frequency or severity, or otherwise reduce these drug product-, API-, or excipient-induced ocular adverse events. Other active agents that may be helpful in these settings are described as well.

The primary underlying polymeric structure most useful (but not the only useful embodiment) in both settings is a cationic graft copolymer. The base structure includes a cationic backbone and grafted hydrophilic side chains. A prime example of such a polymer is poly(L)lysine graft (poly)ethylene glycol. Other molecular structures also accomplish the interactions necessary to confer benefits. Other polymers can use charge, hydrophobic and passivation moieties to accomplish these effects and are addressed herein. The treatment method is through the application to a cell, tissue, organ, organism, or mammal of the aforementioned polymer (whether in solution or not) in an amount and for a duration effective to accomplish the intended beneficial effect. Other polymer configuration with charge, hydrophobicity, and passivation properties including many varieties are contemplated with similar efficacy in ADC protection to corneal cells.

Thus, there is a need to reduce the severity and risks related to new viral disease spreading in human populations and affecting human health.

Further there is a need to reduce the negative impacts related to antibody-drug conjugates, specifically corneal epithelial toxicity.

Inventions disclosed herein thus include methods to treat, mitigate, prevent, reduce, inhibit, or otherwise confer benefit in the setting of topical or exogenous chemicals, excipients, particles, solutes, active pharmaceutical ingredients (APIs) or other materials that induce ocular, conjunctival, corneal, or precursor cell or progenitor cell injury, damage or cell death. Disruption to the normal physiology of the cornea, tears, and ocular surface is also seen in some settings of toxicity. Described herein are methods that provide benefit in these settings as well. Mucopenetration enhancement, tissue penetration enhancement, extended release, and combination drug products can confer additional benefit and are described.

### BACKGROUND OF THE INVENTION

Cationic graft copolymers have demonstrated utility in coating non-biologic surfaces in vitro, in coating medical devices, and for treating dry eye. One example of an effective cationic graft copolymer is Poly(L-lysine)-graft-poly(ethylene glycol)(PLL-g-PEG). PLL-g-PEG is a water soluble co-polymer consisting of a poly(L-lysine) backbone and poly(ethylene glycol) side chains (Sawhney et al. Biomaterials 1992 13:863-870). The PLL chain, which carries multiple positive charges, spontaneously adsorbs onto negatively charged surfaces while PEG is a hydrophilic polymer which serves as a non-binding domain. The PEG moiety passivates a surface while the PLL moiety adheres via electrostatic interaction at the cell membrane or on other charged components of antibodies, viruses, or viral proteins. PLL-g-PEG has been used to passivate in vitro surfaces, experimentally coat medical devices, and has been used as an eye drop for dry eye to lubricate the eye and stabilize the tear film. This invention which goes far beyond the treatment of dry eye is the first identified approach to directly ameliorating ADC corneal toxicity due to tubulin inhibitors among other cytotoxic payloads.

The role of cationic graft copolymers, in particular PLL-g-PEG, in inhibiting viral infectivity and or ADC-related toxicity has neither been considered, studied or reduced to practice prior to this invention. Similarly, effective polymeric molecules have also neither been studied or reduced to practice to Applicant's knowledge in these regards prior to this invention. There are multiple configurations of cationic graft copolymers effective in inhibiting viral infectivity and/or ADC-related toxicity. These alternative molecular approaches are considered and addressed herein.

Methods are also disclosed herein to enhance the efficacy of multifunctional graft copolymers in protecting against on target or off target ADC corneal toxicity, as well as toxicity from other systemically delivered therapies with corneal toxicities (or non-ADC toxicities as well). One approach is the use of enhancers to allow for better trans-mucous and transepithelium delivery such as mucopenetration technology or mucolytics, either or several in combination with multifunctional graft copolymers. Another approach is to utilize a wide variety of sized multi-functional graft copolymers (groups of polymers with lower polydispersity indexes or a wide polydispersity index) to allow some molecules to better penetrate the roughly 50-micron thick corneal epithelium and the conjunctiva, including the perilimbal conjunctiva. Furthermore, disclosed herein are inventions and their embodiments for reducing medicamentosa and exogenous causes for ocular surface irritations including epitheliopathies. Preservatives and some APIs can cause corneal or ocular surface adverse events, and locally applied multifunctional graft copolymers can reduce, mitigate, prevent, treat or inhibit these pathologic changes associated with the application of local or systemic therapies. Methods are described herein to coformulate multifunctional graft copolymers with other actives that may help protect corneal epithelial cells from drug-associated toxicity including ADC induced toxicity.

### SUMMARY OF THE INVENTION

An aspect of the present invention relates to a method for preventing or reducing viral infectivity and viral load exposure and thereby decreasing morbidity and mortality with viral infections such as SARS-COV-2 and other viruses, in particular novel viruses. Charged graft copolymers are safe and effective when applied to at-risk cells and tissues to prevent or reduce infectivity.

An aspect of the present invention relates to a method for preventing or reducing ADC-related drug toxicity to off-target cells and tissue. In particular, the invention is a method to reduce the severity of corneal epithelial cell toxicity in the setting of ADC therapy. Charged graft copolymers are safe and effective when applied to at risk cells and tissues to prevent or reduce ADC-related corneal toxicity. Exposure of human corneal epithelial cells including basal epithelial cells as well as their precursors, wing cells and superficial epithelial cells to the cytotoxic payload carried by an ADC are reduced through the utilization of a treatment with cationic graft copolymers. The exposure of epithelial cells to an effective amount of the graft copolymer in solution is effective at reducing the severity of corneal epithelial toxicity. Precursor cells include any cell that further differentiates or alters into a terminally differentiated cell; included, but not limiting are limbal stem cells and their daughter cells, progenitor cells, wing cells, transient amplifying cells (including early and late), limbal epithelial stem cells, limbal mesenchymal cells (including early and late), basal cells, migrating superficial squamous cells and migrating terminally differentiated cells also benefit from protective therapy. In some embodiments, keratocytes, stromal cells, stroma, Descemet's membrane, and endothelial cells are protected from toxicity. Langerhans cells, melanocytes, fibroblasts and nerve cells are protected. Basement membranes are protected from damage as well. These cells may or may not utilize macropinocytosis. For those that do, there exist specific embodiments directed at these cells to prevent or reduce epitheliopathies. The reduction in severity can be noted on examination findings as a reduction in the microcyst-like epithelial keratopathy, less severe superficial punctate staining, lower rates of epithelial abnormalities, fewer ophthalmic adverse events, reduced rates of decreased visual acuity, fewer complaints of ocular irritation and blurry vision. Reducing ocular toxicity associated with other systemic or local treatments to the eye is also an aspect of the invention including toxicities or adverse events caused by small molecules and biologics.

One aspect of the invention disclosed herein is a method for decreasing viral infectivity by treating tissues that are involved with transfection with an effective amount of a graft or block copolymer with either cationic, hydrophobic, or anionic moieties and a hydrophilic passivation moiety. In one embodiment of the method, the copolymer is PLL-g-PEG. In another embodiment of the method, the graft co-polymer of said formulation comprises a cationic backbone and side chains that are water soluble and non-ionic. In another embodiment of the method, the block co-polymer of said formulation comprises at least one cationic block and at least one water soluble and non-ionic block. In another embodiment of the method, the block co-polymer of said formulation comprises at least one block which is hydrophobic and at least one block which is water soluble and anionic, cationic or non-ionic. In any of the above embodiments, the biological surface to which the formulation of copolymer is administered is a mucous membrane selected from ocular mucosa, oral mucosa, nasal mucosa and respiratory tract mucosa, respiratory tract epithelium, genitourinary mucosa, gastrointestinal mucosa of a subject. In any of the above embodiments, the biological surface to which the formulation of copolymer is administered is a surface of an eye. In any of the above embodiments, the viral infection is selected from coronaviruses, influenzas viruses, Ebola viruses, and novel viruses transmitted through mucous membrane exposure, including, but not limited to the virus is SARS-COV-2. In any of the above embodiments, the graft co-polymer or block co-polymer of said formulation comprises 0.001 to 40% of said formulation. In any of the above embodiments, the graft co-polymer or block co-polymer of said formulation comprises 0.1 to 10% of said formulation. In any of the above embodiments, the passivation effect is based on interference with the SARS-Cov-2 spike protein and ACE2 receptor on at-risk cells. In any of the above embodiments, the therapeutic effect is a general steric inhibition.

In a second aspect of the invention there is disclosed herein a method to decease the adverse events associated with antibody-drug conjugate usage where an off-target uptake pathway that is causing damage to nonneoplastic cells, by applying an effective amount of a copolymer with electrostatic and steric mediating properties applied to cells that are affected by said toxicity. In one embodiment of this aspect, the copolymer is selected from cationic graft, cationic block, hydrophobic graft, hydrophobic block, anionic graft, and anionic block copolymers. In one embodiment of this aspect, the copolymer is formulated in one or more of the following approaches: powders, solutions, suspensions, topical preparations, intravenous preparations, oral preparations, oral rinses, nasal sprays, eye drops. In one embodiment of this aspect, the percentage of the copolymer solution is at minimum 0.01% by weight. In one embodiment of this aspect, the percentage of the copolymer solution is at maximum 40% by weight for solutions and suspensions. In one embodiment of this aspect, the copolymer is PLL-g-PEG. In one embodiment of this aspect, the copolymer is selected from the list of combinations described in the application supra.

In one component of the ophthalmic aspects of this invention, benefit can be seen with topical ophthalmic therapy with co-polymer containing (prescription or not prescription) ophthalmic drug products and/or over-the-counter formulations containing the co-polymer's described herein at a frequency of once daily use (one drop per day per eye), twice daily (two drops per day per eye), thrice or three times daily (three drops per day per eye), four times daily (four drops per day per eye), or up to every hour or more frequent dosage. Dosage may be infrequent as well, at one time per day per affected eye or less. Dosing may be as needed or pro ra nata, as well. The range of dosing frequency needs may be patient and ADC dependent.

In another aspect of the invention there is disclosed herein, a method to decrease ADC related corneal epithelial toxicity by administering a copolymer with bioadhesive and passivation components to cells at risk of off target drug uptake. In one embodiment of this aspect, the copolymer is applied to corneal and conjunctival epithelial cells. In one embodiment of this aspect, the copolymer is PLL-g-PEG. In one embodiment of this aspect, the formulation is a solution for delivery to the subconjunctival space.

In another aspect of the invention there is disclosed herein, a method to decease the adverse events associated with antibody-drug conjugate usage in a human whereby an effective amount of a copolymer with electrostatic and steric mediating properties is applied to cells that are involved in said adverse events. In one embodiment of this aspect, the copolymer is selected from cationic graft, cationic block, hydrophobic graft, hydrophobic block, anionic graft, and anionic block copolymers. In one embodiment of this aspect, the copolymer is selected from polymers in this disclosure herein.

In another aspect of the invention there is disclosed herein, a method to decease the adverse events associated with topical ophthalmic therapy that leads to ocular adverse events such as ocular adverse events mediated by excipients, preservatives, or APIs in a mammal whereby an effective amount of a graft or block copolymer with electrostatic and steric mediating properties is applied to cells that are involved in said adverse events. In one embodiment of this aspect, the copolymer is selected from cationic graft, cationic block, hydrophobic graft, hydrophobic block, anionic graft, and anionic block copolymers. In one embodiment of this aspect, the copolymer is selected from polymers in this disclosure herein.

In another aspect of the invention there is disclosed herein, a method to decrease microcyst-like epithelial toxicity associated with cytotoxins cleaved from ADCs by applying to corneal epithelial cells an effective amount of a copolymer with bioadherence and passivation properties, including a graft or block copolymer with either cationic, hydrophobic, or anionic moieties and a hydrophilic moiety which may serve as a passivation moiety. In one embodiment of this aspect, the copolymer is PLL-g-PEG.

In another aspect of the invention, corneal nerve toxicity is mitigated through these approaches.

In another aspect of the invention there is disclosed herein, a method for decreasing rates and severity of ocular adverse events associated with ADC use by delivering to the eye a copolymer with bioadhesive and passivation moieties including a graft or block copolymer with either cationic, hydrophobic, or anionic moieties and a hydrophilic passivation moiety, prior to initiation of systemic ADC therapy.

Ocular adverse events may include but are not limited to corneal epithelial cell death, superficial punctate keratopathy or keratitis, corneal scarring, corneal infections, micro-cyst like keratopathies, corneal cell injury, corneal cell apoptosis, ocular irritation, ocular foreign body sensation, blurred vision, painful eyes, trouble with vision-related tasks, photophobia, keratopathies, and corneal epitheliopathies.

In another aspect of the invention there is disclosed herein, a method for improving signs and symptoms of ocular adverse events associated with ADC use by delivering to the eye a copolymer with bioadhesive and passivation moieties, including a graft or block copolymer with either cationic, hydrophobic, or anionic moieties and a hydrophilic passivation moiety, after initiation of systemic ADC therapy. Signs and symptoms include(s) visual acuity-related symptoms, visual blur, irritation, redness, and eye-exam findings, but is not limited to these specific signs and symptoms. Vision will be better in co-polymer formulation treated eyes.

In another aspect of the invention there is disclosed herein, a method for reduction of a superficial punctate keratopathy or keratitis associated with ADC use by delivering to the eye a copolymer with bioadhesive and passivation moieties, including a graft or block copolymer with either cationic, hydrophobic, or anionic moieties and a hydrophilic passivation moiety, associated with the initiation of systemic ADC therapy. Deliver may be before, concurrent to, or after initiation of ADC therapy. The benefit is similar for this topical ophthalmic therapy when used with other cornea-toxic drugs mentioned herein. In other words, the specification herein supports claims of benefit for this co-polymer approach broadly, addressing systemic or topical drug or drug products with known or anticipated cornea-toxicity or ocular adverse events particularly those associated with ADC.

An important aspect of this set of inventions is the combination of these methods with tissue or mucopenetration enhancement technologies, extended release approaches, and co-treatment with other actives that may confer benefit, in some cases in coformulations.

Reducing risks of corneal infection are a benefit to co-polymer-based topical therapy in this setting.

In another aspect of the invention there is disclosed herein, a method to reduce ADC uptake into corneal epithelial cells (whether in culture, laboratory models, or in vivo) by exposing said cells to a copolymer with bioadhesive and passivation moieties, including a graft or block copolymer with either cationic, hydrophobic, or anionic moieties and a hydrophilic passivation moiety, prior to exposure to the ADC.

In another aspect of the invention there is disclosed herein, a method to reduce ADC uptake into corneal epithelial cells by exposing said cells to a copolymer with bioadhesive and passivation moieties, including a graft or block copolymer with either cationic, hydrophobic, or anionic moieties and a hydrophilic passivation moiety, after exposure to the ADC.

In another aspect of the invention there is disclosed herein, a method to reduce ADC uptake by macropinocytosis (or pinocytosis more generally) into corneal epithelial cells by exposing said cells to a copolymer with bioadhesive and passivation moieties, including a graft or block copolymer with either cationic, hydrophobic, or anionic moieties and a hydrophilic passivation moiety, prior to or subsequent to exposure to the ADC, using a formulation with an effective percentage based on weight/weight calculations of said copolymer.

In another aspect of the invention there is disclosed herein, a method to reduce ocular adverse events associated with ADCs by treating a patient with a copolymer with bioadhesive and passivation moieties, including a graft or block copolymer with either cationic, hydrophobic, or anionic moieties and a hydrophilic passivation moiety, using an amount to be efficacious. Treatment is local in some embodiments. Local treatment includes topically applied, subconjunctivally delivered, intracamerally delivered, periocular or intraocular delivery. In some embodiments, systemic or dermatologic delivery is beneficial such as for thrombocytopenia prevention or reduction or for peripheral neuropathy reduction.

In another aspect of the invention there is disclosed herein, a method of using copolymers demonstrating electrostatic and stearic interactions at the cellular level, including a graft or block copolymer with either cationic, hydrophobic, or anionic moieties and a hydrophilic passivation moiety, to minimize adverse effects caused by exposure of those cells to factors including SARS-Cov-2, novel viruses, viruses in the setting of an epidemic, ADCs with cytotoxic payloads which can lead to human pathology and morbidity.

In another aspect of the invention there is disclosed herein, a method to reduce ocular toxicity due to systemic exposure of a human to ADCs with tubulin disruptors as the payload by treating the eye with an effective amount of cationic graft copolymer formulation, including a graft or block copolymer with either cationic, hydrophobic, or anionic moieties and a hydrophilic passivation moiety. In one embodiment of this aspect, the cationic graft copolymer is PLL-g-PEG. In one embodiment of this aspect, the treatment is through an eye drop formulation. In one embodiment of this aspect, the formulation is unpreserved. In one embodiment of this aspect, the cationic graft copolymer is PLL-g-PEG in a concentration in an eye drop formulation in a weight/weight range from 0.01% to 5%. In one embodiment of this aspect, less toxic preservatives selected from: sodium perborate, stabilized oxychloro complex, disappearing preservatives, hydrogen peroxide-based preservatives.

In another aspect of the invention there is disclosed herein, a method to reduce dose holds and dose reductions of ADCs in the treatment of human malignancy by reducing corneal adverse events and mitigating ocular safety concerns through a PLL-g-PEG eye drop formulation applied to the eye in an effective amount in an at-risk patient. In another embodiment of this invention, ocular toxicity can be mitigated with local treatment with a copolymer, including a graft or block copolymer with either cationic, hydrophobic, or anionic moieties and a hydrophilic passivation moiety, when said toxicity is due to tear secretion of a cytotoxin and secondary effects on the corneal epithelium. Another aspect is utilizing methods described herein to reduce the risk of poor outcomes or a worse course due to corneal drug related toxicity selected from the effects of: decreasing rates of epithelial erosions, decreasing rates of corneal ulcers, decreasing rates of corneal epitheliopathy, decreasing rates of punctate epitheliopathy, decreasing rates of superficial corneal changes, decreasing rates of corneal stromal inflammation or other changes, decreasing rates of secondary bacterial infection.

For the purposes herein, “adversely affected” may mean but is not limited to any alteration from normal physiology and cell or tissue or organ or organism function, including but not limited to reduced cell survival, initiation of apoptosis, reduced proliferative capacity for cells that normally divide, reduced and improper cell adhesion and migration, alterations in adherence and junctional connections including tight junctions, inflammatory responses to cells or tissue, changes to cell metabolism and catabolismcell bystander damage, changes that lead to pain or discomfort or visual changes or other functional changes for the organ, tissue, or organism, changes that lead to tear film irregularities, changes that lead to fluorescein dye uptake or other vital dyes that are used in epithelial cell assessments (lissamine green, Rose Bengal), damage to the eye, and alterations affecting normal cell heath. Adverse effect(s), adverse event(s), and “adversely affected” are similar and can be used in similar contexts when grammatically, clinically, and technically appropriate. Adverse events include but are not limited to medical problems, cellular health problems, tissue health problems, organ health problems, or organism health problems that happen(s) during treatment with a drug, pharmaceutical or other therapy. Ophthalmic and corneal adverse events in particular are described in detail below. Drug and pharmaceutical may be used interchangeably herein.

For purposes herein, passivation moieties may be and typically are considered hydrophilic moieties, however, alternative moieties may passivate with properties where hydrophilicity does not dominate.

In another embodiment of the invention disclosed herein, an antibody is designed and directed at an ADC with associated corneal toxicity, where the antibody further comprises a passivation moiety, and is delivered to the eye in an amount effective to reduce ocular adverse events such that the antibody with a passivation moiety specifically and directly interacts with a molecular component of the ADC interfering with the activity and/or binding of the ADC with off-target cells, including corneal cells.

Another embodiment of this invention is a method to nonspecifically inhibit corneal toxicity resulting from adverse drug effects where the inhibition is mediated by nonspecific interactions (electrostatic or hydrophobic) of a copolymer described herein, including a graft or block copolymer (a multifunctional copolymer) with either cationic, hydrophobic, or anionic moieties and a hydrophilic passivation moiety, with a cytotoxic agent, and thereby reducing through the effect of passivation moieties the adverse effects of drugs with deleterious effects on the cornea. In some embodiments, the drug with adverse corneal events is an ADC; in other embodiments the drug or pharmaceutical is a small molecule. The pharmaceutical may be a biological, an antibody, and antibody-drug conjugate, a large molecule or a small molecule. A small-molecule drug is any organic compound that affects a biologic process with a relatively low molecular weight, below 900 Daltons. A large-molecule drug is any organic compound that affects a biologic process with a relatively low molecular weight, above 900 Daltons. A large molecule may be an organic compound, a pegylated compound, a protein, a biologic, or a peptide. Peptides may be small or large molecules depending on the molecule weight of the molecule. A biologic or biologic drug in the setting of a pharmaceutical herein is a product that is produced from living organisms or contains components of living organisms.

In some embodiments the corneal toxic drug is selected from the group of: Cationic amphiphilic drugs, amiodarone, aminoquilones, Chloroquine, hydroxychloroquine (Plaquenil), amodiaquine, mepacrine, Tafenoquine, thorazine, tamoxifen, NSAIDs, ibuprofen, indomethacin, naproxen, benoquine, atovaquone, suramin, tilorone, perhexiline maleate, gentamicin, tobramycin, clarithromycin, ciprofloxacin, clofazimine, gold salts, vandetanib, osimertinib, small molecules may be selected from kinase inhibitors, erlotinib, and cytarabine, but not limiting. Corneal vortex keratopathy, corneal deposits, epitheliopathies, verticillata, corneal lines, Hudson Stahl lines, crystal like deposits, stromal inflammation, and corneal opacifications may be reduced or prevented. Limbal stem cell deficiency may be prevented for pharmaceutical agents associated with that limbal stem cell dysfunction toxicity. Pannus, corneal neovascularization, conjunctival epithelialization of the cornea may be reduced or prevented. Without being bound to any particular method, the copolymer passivates the cytotoxic agent found in tears and/or reaching limbal stem cells from the perilimbal circulation.

In another aspect, a method to decease the adverse events associated with any drug's corneal toxicity by treating cells at risk with the copolymer or a solution bathing those cells.

In another aspect, a method to decrease adverse events associated with antibody-drug conjugate usage in the setting of an off-target uptake pathway (or without such an off-target pathway where the toxicity is more direct-either through target uptake, cell receptor similarities to ADC binding sites, or warhead entrance into at risk cells) that is causing damage to nonneoplastic cells, by applying an effective amount of a copolymer to a solution bathing such cells.

The fundamental methodology of decreasing adverse events in cells including corneal cells, associated with their exposure to an antibody-drug conjugate is exposure via solution or suspension, prolonged or acute, of (or to) cationic graft copolymers to the eye and/or corneal cells including precursor epithelial cells, transient amplifying cells, basal epithelial cells, and differentiated epithelial cells, and stem cells (and daughter cells) via topical, local, or systemic approaches (in certain cases) to limit uptake, including macropinocytotic uptake of the ADC. PLL-g-PEG is particularly safe and effective in this setting. Other similar approaches are considered.

Copolymers and multifunctional graft copolymers of the current invention have a bioadhesive moiety as described above (cationic, anionic, hydrophobic) and a passivating moiety (hydrophilic, sometimes chemically inert, where “chemically inert” means without an ability to covalently or electrostatically or using hydrophobicity to significantly react with cells or proteins) such that interaction of viruses and ADCs with the cell compromised by infection or toxicity, respectively, is reduced. All moieties can have varying MW sizes, and combination polymers can be utilized. Formulations with multiple copolymer structures can be utilized. Polydispersity among polymers is known and anticipated in manufacturing and does not reduce effectiveness.

U.S. Pat. Nos. 9,884,074; 9,295,693; 9,283,248; 9,005,596; 8,802,075 discuss graft copolymers and uses for these multifunctional copolymers and are incorporated herein by reference. US patent 2004/0181172 A1 was reviewed; it discusses tear collection for analyzing tears.

Method steps of the invention include to apply the copolymers with bioadhesive and passivation moieties to at risk cells and tissue.

A preferred mode of the invention is through the application of PLL-g-PEG to at risk cells and tissues, preferably before exposure, although its use after exposure confers benefits in reducing severity of adverse findings (secondary epithelial cell damage or repeated exposures or infection or reinfection may be reduced) and reducing the extent of adverse events (minimizing continued uptake by the ADC).

Without being bound to a particular mechanism for ADCs, on-target toxicity includes an antibody/receptor mediated uptake into an epithelial cell that may lead to adverse events that can be also be reduced in this mode (in a local environment particularly). Embodiments of this invention address this approach to ameliorating or mitigating ADC toxicity as well. For example, a copolymer passivates an ADC locally to reduce on-target as well as off target toxicity if a corneal epithelial cell expresses a receptor or protein target on the cell surface that initiates cell uptake. Off target is when the antibody component of the ADC does not directly bind to a receptor or epitope on cell types showing ADC-associated toxicity.

Cells may engulf or pinocytose ADCs through off target interaction, or in some cases, on target receptor interactions in cell types that are not the intended cells for the systemic targeted therapy, and this invention allows an approach to mitigate toxicity in these settings. In fact, in unintended effects on cells that express a target for an ADC, both methods of ADC uptake may coexist (macropinocytosis, and on target binding and internalization). On target binding to an epitope, protein, or antigen expressed by a noncancerous (or corneal, limbal, or conjunctival epithelial cell) may be present because the receptor is also expressed on a cancer cell and the targeting ADC therapy was felt to have benefits in targeting that epitope. For example, a future ADC may target a receptor typically or periodically or rarely or occasionally by a corneal, conjunctival, epithelial, limbal, or transient amplifying, or wing, or basal cell of the eye and show a series of adverse events or ocular toxicity associated with both on target and off target (or macropinocytotic) uptake Note, that ADC uptake need not be macropinocytotic in some embodiments. Fc receptor binding may too play a role, and multifunctional graft copolymers can decrease this Fc interaction. Fc interaction includes but is not limited to Fc gamma receptor FcγR Fc epsilon receptor (FcεR) and Fc alpha receptor (FcαR), and FcμR. These receptors bind IgG, IgE, IgA, and IgM. There are multiple subtypes of these receptors. Multifunctional graft copolymers can interfere with uptake through these receptor binding modalities. Moreover, tisotumab vedotin-tftv, which has significant ocular toxicity) targets tissue factor. Typically, TF (or CD142) is the initiator of blood coagulation and is found on platelets, monocytes, macrophages, and endothelial cells. It is a target for metastatic cervical oncology therapies (the ADC tisotumab vedotin-tftv). Pterygia show tumor-like characteristics, such as proliferation, invasion, and epithelial-mesenchymal transition, and pterygium are found on the surface of the eye. Conjunctival pterygium cells have been shown to express tissue factor, thus implying that there may be some on target interaction with the surface of the eye as well. In some states of corneal health and disease corneal or conjunctival cells may express tissue factor or CD142 to some degree, and this possible on target mechanism allowing another route for some tisotumab vedotin-tftv entry into cells may be through ocular tissue CD142 binding. In these situations, multifunctional graft copolymers limit macropinocytotic entry into cells which represents a contribution to toxicity as well as inhibit CD142 binding and entry into cells, due to copolymer bioadhesive, passivating, and steric interference modalities. Multifunctional graft copolymers are still effective in these cases, especially since the exposure can be modulated by the copolymer delivered topically and locally at the eye. By treating just the eye, systemic efficacy is not affected, and corneal adverse events are mitigated. In fact, the patient will be better able to tolerate tisotumab vedotin-tftv in this setting, thereby improving systemic efficacy while enhancing drug safety. Cetuximab and panitumumab are anti-EGFR antibody/biologics, ocular toxicity associated with these agents and secondary or similar ADCs built off these or similar EGFR binding sites may be mitigated by multifunctional graft copolymers. Non-canonical EGFR endocytosis for the delivery of ADCs into cells can be inhibited in non-target tissue with the local application of a multifunctional graft copolymer. An anti-EGFR (epithelial growth factor receptor), for example, ADC may gain entry into corneal epithelial, conjunctival, or ocular surface cells in or near the front of the eye including limbal stem and daughter cells. Mirvetuximab soravtansine targets folate receptor alpha (FRα), and this receptor can be found on cancerous epithelial cells but has also been noted on rabbit corneal epithelial cells. Mirvetuximab soravtansine has been shown in a clinical report to cause an ocular toxicity. Noncancerous cells that are damaged or killed or exhibit toxicity when exposed to an ADC, whether or not such cells do or may from time to time express an epitope or receptor target for the ADC may show reduced toxicity, damage or rates of cell death when treated with a multifunctional graft copolymer. There may be other toxic drugs with similar and multiple methods of entry into corneal or conjunctival cells, and these potentialities do not change the underlying opportunity for multifunctional graft copolymers to inhibit ADC-related corneal toxicity, or toxicities seen with other biologics or small molecules.

In one embodiment of this invention, a co-polymer eye drop containing formulation may be provided as a kit with a supply of one or more months of eye drop delivery systems possibly along with a chemotherapeutic agent at time of initiation, or consideration for ADC therapy. Kits and eye drops may be delivered to the patient by mail or similar delivery services and may be refilled online.

In some embodiments, the copolymer is supplied as a pharmaceutical composition in eye drop bottles, multi-dose preservative free bottles, standard three-piece bottles, unit dosers/blow fill and seal containers. Eye drop bottle fill volumes are between 1 ml and 30 ml typically. Blow fill and seal containers of various sizes with fills from 0.1 mL to 1 mL (0.5 mL fill, 0.3 mL fill, 0.4 mL fill, 0.7 mL fill) are examples for unit-doser fills. Aerosols, sprays, misters, mechanized or electronic spray bottles, pump spray bottles, liquids for mouthwashes, liquids for consumption, powders, concentrated solutions for dilution, and other commercially available systems to provide consumer goods with pharmaceutical ingredients. A kit can be sold with an eye drop, nasal spray, and mouth rinse. A kit can include a supply of eye drops with an ADC for oncologic or other disease care.

Advantages include that there are currently no methods to reduce the risk of infection by application of a protective material to the mucous membranes, and that is needed in the arsenal against viral disease.

Advantages include that besides supportive care such as warm compresses, bandage contact lenses, and ocular lubricants, there are no therapies that reduce the risk of ADC corneal toxicity based on reducing ADC/epithelial cell interaction.

Based on the results of the trial showing protection of corneal epithelial cells from ADC related damage using graft copolymers such as PLL-g-PEG and other multifunctional graft copolymers, these molecules can demonstrate similar results for the protection of corneal epithelial cells in broader applications. That is the major component of this patent application, all though there are other follow-on inventions and embodiments contained herein.

### BRIEF DESCRIPTION OF THE DRAWINGS

FIG. 1 as expected shows a SARS-COV-2 viral particle in the presence of respiratory epithelial cells. 1. Respiratory epithelial cell nucleus. 2. Respiratory epithelial cell. 3. Cell villi. 4. ACE2 receptor. 5. SARS-COV-2 viral particle RNA. 6. SARS-COV-2 viral particle spike protein. 8. The virus binds to the ACE2 receptor. 9. The virus enters the cell. 10. The SARS-COV-2 virus.

FIG. 2 as expected shows a SARS-COV-2 viral particle in the presence of respiratory epithelial cells and a cationic graft copolymer and the interference imparted by the co-polymer blocking ACE2 related viral cell entry. Although Figure depicts a respiratory epithelial cell, other epithelial cells that may be infected with SARS-COV-2 may be substituted. 1. Respiratory epithelial cell nucleus. 2. Respiratory epithelial cell. 3. Cell villi. 4. ACE2 receptor. 5. A SARS-COV-2 viral particle RNA. 6. SARS-COV-2 viral particle spike protein. 7. Cationic graft copolymer. 11. The virus does not enter the cell because of the interfering effect of the cationic graft copolymer (which is PLL-g-PEG in some embodiments).

FIG. 3 as expected shows an antibody drug conjugate in the presence of corneal epithelial cells. 1. ADC 2. Antibody component. 3. Toxic payload. 4. Corneal epithelial cell. 5. Corneal epithelial cell microvilli. 6. Initiation of macropinocytosis. 7. Completion of macropinocytosis. 8. ADC engaging with macropinocytotic process where ADC is about to be captured by corneal epithelial cell from the extracellular fluid. 9. ADC with toxic payload inside epithelial cell and in lysosome, where the payload will be cleaved from the ADC and released into the cell where it will damage or kill the cell.

FIG. 4 as expected shows an antibody drug conjugate in the presence of corneal epithelial cells and a cationic graft copolymer (which is PLL-g-PEG in one embodiment), note the corneal epithelial cell may be a transient amplifying cell, wing cell, basal epithelial cell, or limbal epithelial stem cell. Drawing, for simplicity, depicts a surface epithelial cell. 1. ADC 2. Antibody component. 3. Toxic payload. 4. Corneal epithelial cell. 5. Corneal epithelial cell microvilli. 6. Initiation of macropinocytosis. 7. Completion of macropinocytosis. 8. PLL-g-PEG on corneal epithelial cell surface. 9. PLL-g-PEG in solution. 10. PLL-g-PEG adhering to ADC at several locations. 11. Lack of ADC with toxic payload inside epithelial cell. Cell injury and death is prevented.

FIG. 5 as expected shows the benefit of cationic graft co-polymer eye drop treatment on an eye in the presence of systemically administered ADC. 1. A schematic of the front of the eye. 2. Schematic of the cornea. 3. Superficial punctate keratitis on the surface of the cornea. 4. Microcyst-like changes in the corneal epithelium noted after a patient has been treated with systemic ADC for cancer. 5. Eye drop containing PLL-g-PEG being administered to the eye of a patient receiving systemically administered ADC for cancer. 6. PLL-g-PEG in solution in the eye drop. 7. Cornea with much healthier appearing surface in ADC treated patient and PLL-g-PEG eye drop treated patient. 8. Fewer microcyst-like changes in PLL-g-PEG eye drop treated patient. 9. Less superficial punctate keratitis in PLL-g-PEG eye drop treated patient, despite ADC on board systemically.

FIG. 6 ADC uptake in human corneal epithelial cells in presence of increasing amounts of PLL-g-PEG. Dose response of PLL-g-PEG in reducing ADC uptake in human corneal epithelial (HCE-T) cells is demonstrated.

FIG. 7 Experimental design of ADC uptake study.

FIG. 8 Pilot evaluation comparing PLL-g-PEG with EIPA which is not viable as an intervention because it is toxic (positive control).

FIG. 9 Plots of fluorescence intensity indicating ADC uptake in corneal epithelial cells

FIG. 10 Bar graphs of fluorescence intensity indicating ADC uptake in corneal epithelial cells

FIG. 11 Bar graphs of log transformation indicating ADC uptake in corneal epithelial cells

FIG. 12 Cell surface-bound and internalized cytosolic ADC was detected by green fluorescence

FIG. 13 as expected shows an antibody drug conjugate and a small molecule inhibitor of macropinocytosis in the presence of corneal epithelial cells and a cationic graft copolymer (which is PLL-g-PEG in one embodiment), note the corneal epithelial cell may be a transient amplifying cell, wing cell, basal epithelial cell, or limbal epithelial stem cell. Drawing, for simplicity, depicts a surface epithelial cell. 1. ADC 2. Antibody component. 3. Toxic payload. 4. Corneal epithelial cell. 5. Corneal epithelial cell microvilli. 6. Initiation of macropinocytosis. 7. Completion of macropinocytosis. 8. PLL-g-PEG on corneal epithelial cell surface. 9. PLL-g-PEG in solution. 10. PLL-g-PEG adhering to ADC at several locations. 11. Lack of ADC with toxic payload inside epithelial cell. 12 Small molecule macropinocytosis inhibitor. 13 Lack of ADC with toxic payload inside epithelial cell and presence of macropinocytosis inhibitor with different mechanism to prevent epithelial cell uptake of ADC than PLL-g-PEG. Cell injury and death is prevented.

FIG. 14 as expected shows the benefit of cationic graft co-polymer eye drop treatment on an eye in the presence of systemically administered ADC. 1. A schematic of the front of the eye. 2. Schematic of the cornea. 3. Superficial punctate keratitis on the surface of the cornea. 4. Microcyst-like changes in the corneal epithelium noted after a patient has been treated with systemic ADC for cancer. 5. Eye drop containing PLL-g-PEG being administered to the eye of a patient receiving systemically administered ADC for cancer. 6. PLL-g-PEG in solution in the eye drop. 7. Cornea with much healthier appearing surface in ADC treated patient and PLL-g-PEG eye drop treated patient. 8. Fewer microcyst-like changes in PLL-g-PEG eye drop treated patient. 9. Less superficial punctate keratitis in PLL-g-PEG eye drop treated patient, despite ADC on board systemically. 10. Mucopenetrating agent in coformulation eye drop to enhance therapeutic effect of PLL-g-PEG getting through the mucin of the eye into corneal epithelial cells and deeper into the corneal tissue so as to benefit limbal stem and daughter cells, and corneal nerves and basal epithelial cells and transient amplifying cells and wing cells.

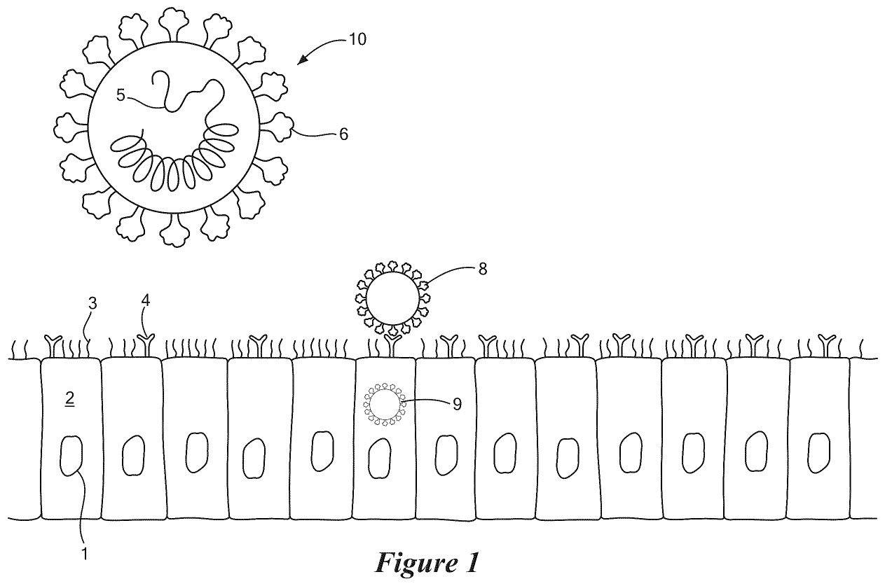

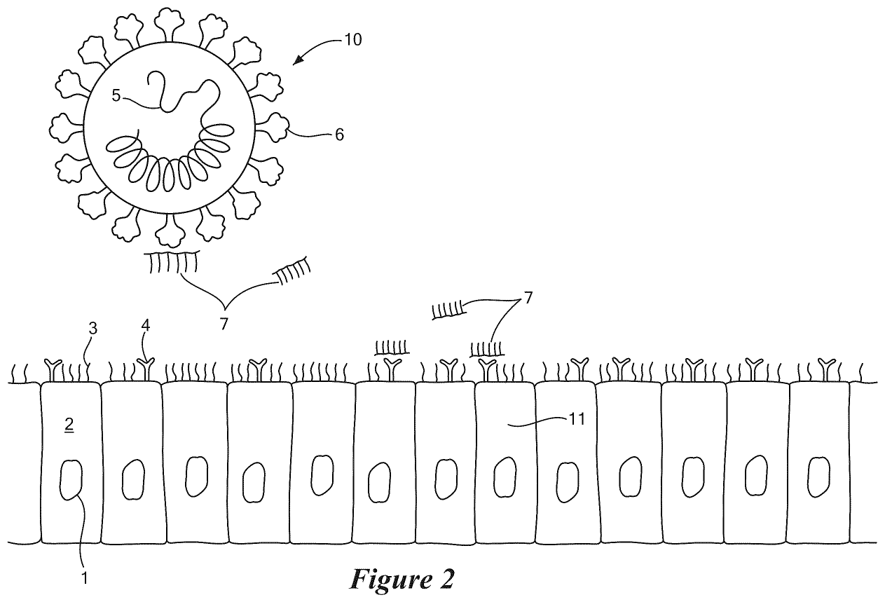

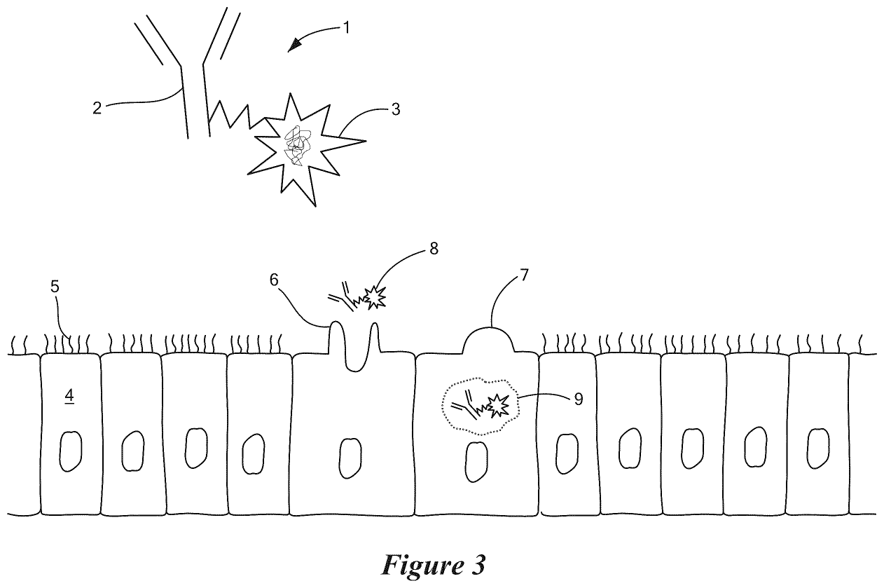

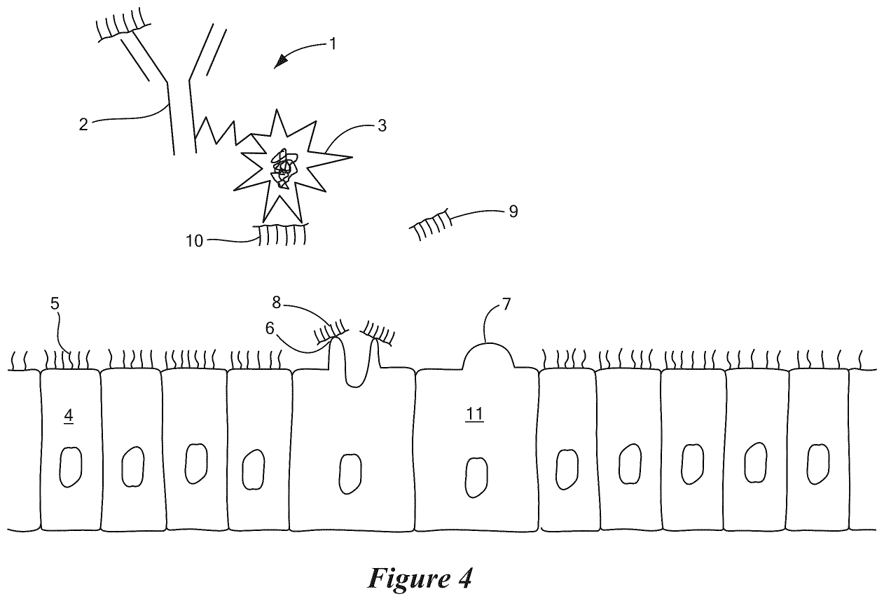

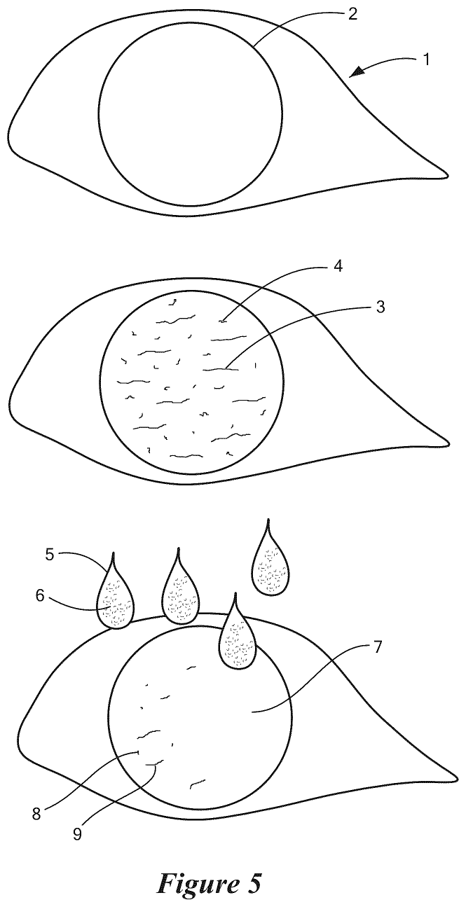

### DETAILED DESCRIPTION OF THE INVENTION

It has now been found that a graft co-polymer having a positively charged moiety and a hydrophilic moiety or a block co-polymer having a positively charged or moiety and a hydrophilic moiety are effective in two important aspects of human health.

First, these polymers are effective at reducing viral infectivity and infection severity if infection occurs by contact with epithelial surfaces, as well as other exposure methods. It is known that viral dose may relate to the severity of an infection on a dose-response type basis. Even if an infection does occur, reducing the number of viral infections in cells will have beneficial effects on disease course (an intervention that is 100% effective is rare).

Second, these polymers are effective at reducing antibody-drug conjugate (ADC) toxicity on nonneoplastic cells. These polymers are able to interfere with off-target cell uptake of ADCs with cytotoxic payloads into cells that do not express the ADCs target receptor. In some embodiments and with certain ADCs interfering with on target receptor uptake is accomplished as well, especially in the local setting where there is no meaningful negation of efficacy targeting other non-ophthalmic tissues. The mechanisms or=f electrostatic and steric interference remain. Through topical exposure, these polmers also can reduce uptake by locally treating tissue to passivate the ADC. (Copolymer may be in the lining mucosal solution or extracellular space to interfere with off target ADC uptake.) Specifically, regarding off target uptake these cationic graft or cationic block copolymers interfere with macropinocytosis of an ADC by a human corneal epithelial cell, including a basal epithelial cell, limbal stem cell, basal stem cell, wing cell, or superficial epithelial cell. This reduction of exposure of the internal cell body, or cytoplasm, including lysosomes, and in some embodiments to tubulin forming components provides a benefit to the cell and organism.

Without being bound to theory, the method of interference is that the graft or block copolymers passivate the surface of the cell to be affected and/or the ADC or virus such that cell uptake is prevented or reduced. The cationic graft copolymer or other polymeric embodiments interact with a biological surface and/or the surface of a virus or an ADC. Interference occurs at the cell membrane and on the biologic in some embodiments.

The key discovery is the ability of a cationic graft copolymer such as PLL-g-PEG to be effective in this setting. It is safe and well tolerated, and highly efficacious. There are multiple embodiments of PLL-g-PEG that are effective, and these polymers are tunable. In some embodiments alternative polymeric structures are effective including block and dendrimer polymers—both types can also be considered multifunctional copolymers.

Definitions (other and additional definitions are embedded in text as appropriate)

“Decrease” or “reduce” means: to make smaller or less in amount, degree, or size. This reduction as applied herein may be understood by those skilled in the art. In addition, for various embodiments, reduced or decreased means a numerical improvement vs. a comparative treatment group that can serve as an untreated control as applicable. The numerical reduction or decrease may be 1%, 3%, or 5% vs. the untreated control group in some embodiments. The reduction may at least 5% in some embodiments. The reduction may be at least 10% in some embodiments. The reduction may be at least 20% in some embodiments. The reduction may exceed 20% in other embodiments

By “off target uptake” is meant the entry of an ADC into a cell using a mechanism different or not completely dependent on the antibody—cell receptor interaction for which the targeted therapy was designed. “On target” is when the ADC binds a receptor on a non-neoplastic cell.

“New viruses” can mean new to a host (human or otherwise), or not encountered in humans before.

A host is the living being that the bacteria, virus, protozoan, or other disease-causing microorganism normally resides in.

“Biological Surface” means surface of a cell, tissue, bodily organ, whether it be exposed to the external environment, or internal to the body. For example, the surface of the eye includes the epithelial cell covered cornea and conjunctiva, as well as the posterior tenons layer and sclera; the epithelial layers of the gastrointestinal tract or the skin are included, membranes such as mucous membranes, including oral, nasal, respiratory, and vagina mucous membranes. Other surfaces include the capsules of organs such as the spleen and liver, and the outermost aspect of bone, cartilage, and muscle. Copolymers described herein may also interact at the surface of the virus and ADC to confer benefit.

“Formulation” means a solution, suspension, powder, spray, rinse, eye drop, administered to a cell, tissue, organ, or mammal to be treated that includes the necessary components to enable the beneficial effect from the graft or block copolymer to take place. A formulation may or may not include an active pharmaceutical ingredient. For the purposes of this invention, the graft copolymer may or may not be considered an active pharmaceutical ingredient by regulatory authority terminology. The eye drop formulations may have various percentages of the copolymers (an effective range), may have a pH between 3.9 and 9.9, may have an osmolality between 150 and 400, may have a viscosity between 1.0 and 15 cP. Viscosities can go much higher in some embodiments. Formulations are safe for subconjunctival injection. Coformulations have more than one agent or molecule that confer treatment benefit in the settings described herein

“Microtubule disruption” is considered a cytotoxic effect of particular interest herein. Examples of these “cytotoxic agents” include but are not limited to MMAF (Monomethyl auristatin F (MMAF) is an antitubulin agent agent that inhibits cell division by blocking the polymerization of tubulin), MMAE (monomethyl auristatin E), DMF (dimethylformamide), Maytansine, aurastatins, DM4 (Ravtansine) andDM1 (Mertansine). Other cytotoxic agents are listed elsewhere, some in combination with antibodies. All are included, and unknown cytotoxic agents as well, are included in various embodiments.

The “cytotoxic payload” may also be called a “warhead”. Linkers are used to join the antibody with the warhead. Linkers are cleaved by intracellular and sometimes extracellular enzymes. In particular, linkers are cleaved in lysosomes releasing the warhead within the cell. Extracellular fluid or enzymes in tears may in some instances cleave a linker a release a warhead. Warheads may thus directly enter cells or, warheads may leak out of cells and cause a bystander effect (damage cells nearby that did not uptake the ADC). Any linkers, and there are many (both known and unknown) are considered in embodiments of this invention. Linkers nay be specific to cells and treatment situations.

As used herein, the term “pharmaceutical composition” refers to a composition in which an active agent is formulated together with one or more pharmaceutically acceptable carriers. The composition is suitable for administration to a human or animal subject. The active agent is present in unit dose amount appropriate for administration in a therapeutic regimen that shows a statistically significant probability of achieving a predetermined therapeutic effect when administered to a relevant population.

When discussing target binding, the term epitope can be used to reflect a protein structure, receptor, or antigen present or expressed on a mammalian cell surface that an ADC may or may not bind directly to.

Nonneoplastic cells are also considered noncancerous cells.

Formulations of copolymers in the present invention include embodiments using many types of formulations including gels, lotions, creams, ointments, sprays, wipes, salves. Formulations may be preserved or unpreserved. Formulations may be monophasic or multiphasic. Without limitations, phasic formulations have varying amounts of different components to affect efficacy and duration of activity. Some formulations have prolonged activity, others have shorter activity. In general, a single application provides at some meaningful duration of time protection. A “meaningful duration of time” for the purposes herein means up to 0.1 minute, up to 0.5 minute, up to 1 minute, up to 15 minutes, up to 30 minutes, up to one hour, up to two hours, up to three hours, up to four hours, or up to six to twelve hours typically. In some embodiments, duration is longer than 12 hours. Extended-release formulations will increase the duration of action. The duration of activity value can be shorter or longer regarding viral protection and ADC toxicity reductions, as determined in development. For methods of ADC toxicity attenuation and mitigation, dosing may range from daily to hourly depending on the formulation and/or payload of the ADC, patient status, and underlying condition, and formulation. The use of these approaches in vitro (as well as in vivo) has practical value and may be shorter or longer depending on the in vitro model.

Animal data for other uses with small interfering RNA molecules has shown PLL-g-PEG can be tolerated intravenously. PLL-g-PEG is well tolerated locally and has thus particular value herein. By “tunable” it is meant that a cationic graft copolymer can be prepared, and remain useful, in various ways. For example, the length of the PLL chain, the graft ratio to mer in the polymer, and the length of the hydrophilic side chain are tunable. By way of example, the PLL-g-PEG molecules utilized in the experimental proof, or reduction to practice for this invention include, PLL (15,000 to 30,000 Daltons)31 graft (3.5 ratio)−PEG (5,000). Alternative molecule weights and graft ratios can be equally, more so, or slightly less effective. For the purposes of this invention, all PLL-g-PEG variations are effective and are referred to as PLL-g-PEG. Mimetics can be similarly tunable. Because PEG is a hydrophilic molecule, it has been used to passivate microscope glass slides. Polyethylene glycol has a low toxicity and is used in a variety of products. The polymer is used as a lubricating coating for various surfaces in aqueous and non-aqueous environments.

Regarding specifics of the PLL-g-PEG. Polylysine (PLL) facilitates the attachment of proteins and cells to surfaces. The PLL may be larger than 30,000 Daltons even up to 60,000 Daltons or higher or it may be smaller than 15,000 Daltons or even 5,000 Daltons or below in some embodiments. Ranges of polydispersity are acceptable, as well, at times even desired. The polydispersity index is a measure of the heterogencity of a sample based on size or the breadth of the molecular weight distribution. Polydispersity can occur due to size or MW distribution of polymers in a sample. A Polydispersity index (PDI) of 1.0 to 5.0 is considered acceptable. In some embodiments, the PDI range is 1.0 to 2.0. In other embodiments, the range is 1.0 to 3.0. In certain embodiments, the PDI range is 1.0 to 3.5. Sometimes, PLL can be reported as a single, average molecular weight (e.g. 20,000 Daltons). Larger or smaller PLL molecules are effective in this setting. The preferred configuration in one embodiment is a mean PLL molecular weight between 10,000 and 40,000, but larger or smaller PLL molecular weights are effective as are smaller sizes to enhance transcorneal or transconjunctival movement. Polydispersity indexes can vary and still be effective. The number of lysine (L lysine, D Lysine, alpha, or epsilon polylysine but not limited to) repeat units can be 50 to 200 in some embodiments, longer or shorter (fewer or more mers) are acceptable. In some embodiments, the number of lysine repeat units is 135. In certain embodiments, the number of lysine repeat units is 100 to 150. The grafting ratio is optimally 3.5 or 4 PLL:PEG, however a reasonable preferred range is 2 to 6. Above 6 to 7, 8, 9, or 10 is acceptable in some embodiments. A preferred embodiment is a PLL:PEG graft ratio of 2.5 to 4.5. The upper limit is simply the point at which tolerability is reduced. It is anticipated efficacy will decrease as the graft ration falls below 2, however, 1.1 is probably the lower limit of optimal benefit from the mechanism with the cationic PLL chain utilizing charge for bioadherence. Hydrophobic chains and other configurations may have different success rates and are also tunable. The hydrophilic moiety, in this case PEG can also have different molecular weights. PEG 5,000 Daltons is one preferred embodiment, but PEG 2,000 is also effective. Ranges of PEG molecular weights that in this specific molecule's use are effective include PEG 1000 to PEG 20,000. Ranges and variable polydispersities are acceptable (1.0 to 2.0). The molecular weight of PEG can also be represented by the number of ethylene glycol repeat units. In one embodiment, the number of ethylene glycol repeat units is 22 to 250. In some embodiments, the number of ethylene glycol repeat units is between 50 to 150. In some embodiments the number of ethylene glycol repeat units is greater than 250. These factors are tunable, and the same or similar effectiveness can be seen with carrying PLL size, graft ratio, and PEG size. Optimal, but not limiting, configurations are described. Mixtures of different base PLL-g-PEG copolymers are acceptable. Multiple different linkers of mPEG to PLL are acceptable. Mimetics, as outlined herein can be addressed similarly with graft ratio, backbone size, and hydrophilic/passivation moiety size.

A cationic graft copolymer is a multifunctional graft copolymer.

Multifunctional graft copolymer topical ophthalmic formulations can protect corneal epithelial cells, the cornea, and/or the ocular surface generally. Any aspect of the ocular surface can be protected using these multifunctional graft copolymers in including PLL-g-PEG. When multifunctional graft copolymers are used, the term includes PLL-g-PEG as one embodiment, and other cationic backbone polymers with hydrophilic or non-ionic side chains as well, without limiting the breadth of the invention. Furthermore, in the situations where applicable, block copolymers can be utilized and are identified and claimed herein for all interventions and approaches. Additionally, included herein are either in isolation or alone in formulation or in combination with multifunctional graft copolymers include other biocompatible molecules that can be utilized to enhance the protection of corneal epithelial cells from ADCs such as mucopenetrating technologies including polymers, bioadsorbance or absorbance enhancers, or tissue penetrators. Mucopenetration enhancement can help drive multifunctional graft co-polymers more deeply into corneal and conjunctival tissue to help promote treatment benefit or efficacy. Likewise, this approach of including penetration enhancers in a formulation for the eye can be used to increase the penetration of other active agents that may have anti-macropinocytotic or protective effects on the corneal, conjunctival or limbal stem or daughter cells and thereby enhance efficacy or beneficial effect. To be clear, coformulations with tissue penetration enhancers with both a multifunctional graft protein and another active agent that can reduce ADC toxicity, or either alone is one embodiment of this invention.

Mucopenetration enhancers are defined herein as a molecule or set of molecules that help multifunctional graft copolymers penetrate deeper into corneal or conjunctival tissue. Mucopenetration can be considered tissue penetration in some cases for alternative, but similar embodiments. Tissue penetration enhancers may be used synonymously herein as the cornea and conjunctiva are key tissues under discussion and these tissues have mucin on their superficial surface. Mucopenetration enhancers are mucopenetrating technologies and vice versa. Bioadsorbance or absorbance enhancers are mucopenetration enhancers in some situations as used herein. Mucpenetrating technologies may be considered mucopenetrating agents. “Mucopenetrant” can be considered a mucopenetration technology, mucous membrane penetration technology, mucin or tissue penetration enhancer, mucopenetrating agent or a tissue penetrating technology, as applicable.

Furthermore, there are set of therapeutic agents shown to have an ability to inhibit the process of macropinocytosis. These molecules were studied for various reasons, and those with clinical potential for safety and efficacy include: auranofin, flubendazole, imipramine, itraconazole, phenoxybenzamine, terfenadine and vinblastine. In addition, protein kinase c is activated in macropinocytosis and nicotinamide adenine dinucleotide phosphate oxidase (NADPH) oxidase 2 can mediate components of macropinocytosis. Families of molecules not limited to the above-named compounds may have inhibitory effects on macropinocytosis that could serve useful in treating or preventing corneal and conjunctiva toxicity associated with ADCs. Not only are the above-mentioned molecules potential novel therapeutics in this regard, but other molecules, even those as yet undiscovered and those with known systemic or oral bioavailability issues. For example, local topically delivered agents with poor bioavailability or off target toxicities may show potential with topically delivered agents. These agents may be used alone or in combination with the host of polymeric approaches to inhibiting macropinocytosis or otherwise protecting corneal and conjunctival cells with local delivery. Combination formulations with more than one active (e.g. a multifunctional graft copolymer and an alternative molecule showing inhibitory activity, or other cytoprotective activities in the setting of ADC induced ophthalmic toxicity) are an embodiment of this invention.

Topical sodium thiosulfate, via mechanisms of protection against cyanide poisoning and those for effects on dry eye (reducing the expression of molecules associated to inflammation), may have benefit in ADC-related toxicity. The use of multifunctional graft copolymer with sodium thiosulfate in combination to reduce ADC related corneal toxicity, is an embodiment of this invention for corneal protection from ADC toxicity. It can be considered a coformulation.

Different therapeutics via different mechanism are embodiments of this set of invention whereby approaches to mitigating corneal and ocular toxicity associated with ADC, small molecule or biologic toxicities. One of these approaches is in the use of peroxisome proliferator-activated receptor gamma and peroxisome proliferator-activated receptor alpha. Peroxisome proliferator-activated receptor (PPAR) agonists can help protect corneal epithelial cells and corneal nerves in the setting of ADC toxicity. Including a PPAR agonist in a coformulation or use independently in this setting of mitigating toxicity is an embodiment. Another potential method to mitigate ocular adverse events associated with oncologic therapy includes targeting proteinase-activated receptor (PAR)-1 and −2. These receptors potentially modulate inflammation and serve as a target for a corneal therapy in this regard.

There are certain molecules, such as saponin, Triton X-100 and Tween-20, that permeabilize cell membranes. Likewise, there are molecules that can help molecules penetrate mucin or mucous membranes. There are similarly molecules that can help molecules penetrate epithelial tissue. Nonlimiting examples include mucous penetrating nanoparticles, selectively sized nanoparticles (200-400 nm) with noncovalent coatings. with, two key attributes to help the drug particles, and in this case multifunctional graft copolymers, penetrate through mucus barriers and significantly increase exposure to the corneal epithelial cells and to cross the conjunctiva and epithelium of the cornea. For example, a formulation with some or all of the following may prove beneficial: glycerin, sodium citrate dihydrate, Poloxamer 407, sodium chloride, EDTA disodium dihydrate, citric acid, and water for injection. Other pluronics or polaxamers can be substituted here. Likewise, surfactants can be added to formulations to assist in efficacy and target tissue engagement. Similarly, acetylcysteine can be used in the current embodiments of the invention to help thin and loosen mucus to allow the multifunctional graft copolymers to get deeper into ocular tissue and enhance efficacy. Other molecules that can enhance multifunctional graft copolymers efficacy in corneal and conjunctival cell and tissue protection, including limbal stem cells and daughter cells are claimed. In no means limiting, additional potential molecules can be used in combination with or prior to or just after instillation with a multifunctional graft copolymer. Exemplary studies demonstrate clinical benefit of the combination approach. The protease of C1 esterase inhibitor (StcE) can be used in this setting. Other microbial derived products are contemplated and included herein. These and other approaches include: enzymatic degradation, proteolytic enzyme usage, trypsin, papain, and bromelain (which can be naturally found in the digestive tract, papaya, and pineapple, and there by provide opportunities to leverage natural products and natural ingredients), other mucolytics, approaches that decrease mucous viscosity, DNAase, disulfide bond cleavage, charge shielding (in addition to and including what multifunctional graft copolymers can do), increased electrostatic repulsion, can be used. In addition to enzymatic degradation, other chemical methods are utilized to alter mucus structure to allow for both better drug delivery and treatment of diseases. In addition to mucolytics, expectorants, and mucokinetic agents are an embodiment herein with the use of multifunctional graft copolymers to help minimize the mucous as a barrier to efficacious protection of corneal cells, conjunctival cells, precursor cells in the topical ophthalmic setting. There are also similar opportunities for gastrointestinal, oral, nasal, respiratory, vaginal, and other mucous membrane interventions to help protect epithelial cells and tissues in these categories from toxic exogenous effects and to decrease adverse events and mitigate disease. N-acetylcysteine (NAC), a mucolytic, reduces mucin cross-linking by cleaving disulfide bonds (108), which constitute important intermolecular and intramolecular mucin cross-links. NAC decreased mucus viscosity, increased pore size, and increased the diffusion rate, S-carboxymethlycysteine (carbocisteine), affects sialyltransferase activity in goblet cells resulting in increased sialylated mucin, Phosphatidylglycerol distearoyl, phosphatidylglycerol dipalmitoyl. Also included are chemical agents able to disrupt ionic interactions to alter mucin cross-links. Included are chelation of calcium ions approaches from mucus to results in the rapid swelling, hydration, and dispersion mucin. Calcium chelators bicarbonate and ethylene glycol-bis(β-aminoethyl ether)-N,N,N′,N′-tetraacetic acid (EGTA) can reduce viscosity of mucin aggregates and help disperse of mucin fibers. Guaifenesin and guanidinium hydrochloride are also considered herein. Urea may be used herein and other chaotropic agents including carbamide, and other synonyms and similar structures. Carboxymethylcellulose and polysorbate-80 can be included in formulations and treatments to enhance the effects of multifunctional graft copolymers for protective approaches. Thiolated chitosans can be used as well and are claimed as an embodiment. Chondroitin sulfate/dextran, polycarbophil/edetate disodium dihydrate and sodium chloride, and a host of other co-formulating opportunities as are described and claimed herein. Iontophoresis and electrical stimulation can also help large molecules permeate more deeply into tissue. Molecules and approaches described in Carlson T L, Lock J Y, Carrier R L. Engineering the Mucus Barrier. Annu Rev Biomed Eng. 2018; 20:197-220. doi: 10.1146/annurev-bioeng-062117-121156, in combination or temporally related in treatment with multifunctional graft copolymers to protect epithelial cells, tissue and progenitor cells are included as embodiments herein. Collectively, the technologies, molecules, agents, and approaches supra are considered mucopenetration enhancers. Mucopenetration enhancers are also mucous membrane, mucin layer, or glycocalyx penetration enhancers. Mucous, cell, or tissue penetrating technology encompasses, but is not limited to the approaches of increasing multifunctional graft copolymer treatment benefit as described above, in some embodiments through improved pharmacokinetics or pharmacodynamics or residence time or target tissue exposure-especially an ability to drive large molecules deeper into exposed tissues. These technologies may be used for tissue loading or chronically. Or can be separate formulations which work synergistically.

Many of the approaches described herein can be used in topical formulations intended to prevent, treat, protect, heal or cure corneal wounds, ulcers, and scars as well as mitigate corneal adverse events. The treatment reduces the risk and rate of secondary infection.

Mucus membrane penetrating agents, however, may not be required due to damaged mucous protection in some disease states. An embodiment of the current invention is the use of these multifunctional graft copolymers in conditions with alterations of the mucus membrane such as dry eye and corneal epithelial toxicity induced from exogenous agents that naturally allow for better tissue pharmacokinetics and levels of the multifunctional graft copolymers in the desired anatomic locations (e.g. the surface of the cornea, into the corneal epithelium, subconjunctival space, location within the mucous membrane, in the tear film, and on the conjunctiva). In other embodiments, co-formulations with permeability and penetration enhancers is described. Sequential delivery is also an embodiment, whereby pretreatment with a mucous penetrating agent or mucous disruptor can enhance the efficacy of multifunctional graft copolymers in treating or preventing corneal epithelial disease caused by exposure to exogenous sources such as pharmaceutical or biologic therapy (local, topical, or systemic), including toxicities caused by APIs and/or excipients or preservatives.

Furthermore, ethyl-isopropyl amiloride (EIPA) has been shown by others to inhibit macropinocytosis. EIPA, however, is toxic to cells as demonstrated in experiments where corneal cell death was manifest following low dose exposure, as shown by others. The use of multifunctional graft copolymer with safe and tolerable amounts EIPA or other agents that reduce macropinocytosis in combination with multifunctional graft copolymers to reduce ADC related corneal toxicity is an aspect of the current set of inventions. Likewise, the macropinocytosis inhibitors imipramine, phenoxybenzamine and vinblastine can be used with multifunctional graft copolymers to mitigate ADC induced toxicities. Other macropinocytosis inhibitors include but are not limited to: Flubendazole and other anthelmintics, terfenadine and other histamine H1-receptor antagonists, itraconazole and other antifungal medicines, other α-Adrenergic antagonists, auranofin and other anti-rheumatoid arthritis agents, other tricyclic antidepressants. These agents may be used in low dose with multifunctional graft copolymers to enhance ADC induced protection conferred by multifunctional graft copolymers. Amounts utilized in these co-formulated products will be safe and effective.

Tissue penetration enhancers to increase the efficacy include but are not limited to bioadhesives, including hydrogels and other agents like carbopols, polyacrylic acids, chitosan, etc., and penetration enhancers, including different surfactants, calcium chelators, etc. cationic graft copolymers themselves can increase the residence time for topical formulations, formulations that further improve the residence time may prove beneficial. Penetration enhancers or absorption promoters can also be used in multifunctional graft copolymer formulations—for the eye, skin, or other mucosal or epithelial surfaces. Penetration enhancers can be selected from the groups as follows: calcium chelators, surfactants including ethylenediaminetetraacetic acid (EDTA and other aminopolycarboxylic acids, surfactants, bile acids and salts, lysophosphatidilo lipids, preservatives, cetylpyridinium chloride and BAC, glycosides including saponin, digitonin, fatty acids, azone, ionophores, and cytochalasins, without limiting other molcules and structures. Ionotophoresis can also be used with or without external electrical stimulation to drive multifunctional graft copolymers deeper into ocular tissue. Methods that enhance transcorneal drug delivery are embodied herein. Polymers can also improve bioadhesive aspects for these formulations and ultimately enhance efficacy for reducing medicamentosa and ADC related or systemic drug related toxicity. Polymers such as macromolecular hydrocolloids with hydrophilic functional groups that can form hydrogen bonds (such as carboxyl, hydroxyl, amide, and sulfate groups) are included. Included but without limitations are hydroxypropyl cellulose, hydroxypropyl methylcellulose (HPMC) as the non-ionics; chitosan, DEAE-dextran as the polycationics; polyacrylic acid (PAA) derivatives, e.g., carbopols, polycarbophils, carboxymethylcellulose (CMC), and poly-anionics. Xanthane, carrageenan, and bioadhesive polysaccharides are included. Peptides may be used to enhance penetration. Hyaluronic acid and similar chemical structures and derivatives are included. Viscoelastics and viscoadhesives are included, and see this reference for additional details utilized in formulations to improve the efficacy and effect for the multifunctional graft copolymer in mitigating ocular AEs or other adverse events: Indu Pal Kaur & R. Smitha (2002) Penetration Enhancers and Ocular Bioadhesives: Two New Avenues for Ophthalmic Drug Delivery, Drug Development and Industrial Pharmacy, 28:4, 353-369, DOI: 10.1081/DDC-120002997 for a more detailed list of penetration enhancers for co-formulations with multifunctional graft copolymers to improve efficacy in reducing ADC toxicities. Additional enhancers include nanotechnology enhancers, nanotechnology delivery systems, terpene and techniques used for transdermal penetration enhancers, including: alcohols, short chain alcohols, long chain alcohols, amides, azone, esters, alkyl esters-ethyl acetate, benzoate esters-octyl salicylate, fatty acid esters-isopropyl myristate, glycols-propylene glycol (pg), glycol ethers, fatty acids, pyrrolidones, sulphoxides, surfactants, anionic surfactants, cationic surfactants, non-ionic surfactants. See for details in this reference for formulations to be embodied in various formulations for the benefit of reducing systemic or ocular AEs or peripheral neuropathy: Lane, Majella E. “Skin penetration enhancers.” International journal of pharmaceutics 447.1-2 (2013): 12-21. Furthermore, mucoadhesive formulations (polymer-based solutions, viscous gels, and mucoadhesive micro—and nanoparticles) are an embodiment of the improved safety and/or efficacy formulations. Including dextran, glycerol, and hydroxypropylmethylcellulose in a chondroitin sulfate emulsion can be used with multifunctional graft copolymers as well for formulations with attributes that are clinically beneficial.

Block copolymers listed below in this application including poloxamers may enhance the efficacy of multifunctional graft copolymers as an approach to improve exposure of tissue or cells to multifunctional graft copolymers.

Of course, as mentioned, PLL-g-PEG is the primary, but not only multifunctional graft copolymer conferring such protective benefits. Cationic graft copolymers and mimetics thereof are included. The term “mimetics” means there is a structural mimicry of effect using different configurations of and base moieties to generate similar in vitro and in vivo and in clinic safety and efficacy. A mimetic can also mean a polymer which biologically mimics the action or activity of some other polymer. Mimetics applies to both the multifunctional graft copolymers and their effects when referenced in that context, and also mimetics that mimic the behavior or molecules that enhance tissue, epithelial layer, conjunctiva, and mucin or mucous penetration and or permeability to polymers are considered in coformulations.

Coformulations can be considered (but are not limited to this definition) drug product formulations including topical ophthalmic drug product formulations that include more than one class of molecules beneficial to the eye or cornea in protecting or enhancing the protection of the conjunctival cells, corneal epithelial cells, limbal stem cells, daughter cells including transient amplifying cells, wing cells, basal cells, epithelial cells, superficial epithelial cells, and corneal nerves. Thus, coformulations may include two actives that protect cells from ADC damage for instance. Formulations with one active and a mucopenetration enhancer for example is a coformulation. The coformulation may have a multifunctional graft copolymer and a mucopenetration enhancer. There may be three different classes as well. Concentrations by a weight/weight may range from 0.000001% for very potent agents to 40% for those less potent.

Medicamentosa is a chemical irritation of ocular and/or adnexal tissues by a topically applied drug or cosmetic, or by environmental or occupational substances.

Embodiments of the present invention include a set of inventions to protect patients (human and mammals) from medicamentosa and other toxicities to the ocular surface associated with topical ophthalmic therapies. In particular, preservatives can cause or lead to poor tolerability to ophthalmic medications. These preservatives include but are not limited to: preservatives such as Polixetonium, polyquaternium-42, Polyquaternium-1, Polyquat, Alkyl-hydroxy benzoate preservatives, parabens, hydrogen peroxide, benzalkonium chloride (BAK or BAC), cetylpyidimine chloride, cetalkonium chloride, sodium perborate, Purite, disappearing preservatives, polyhexamethylene biguanide (PHMB), chlorobutanol, benzododecinium bromide, “Ionic buffered systems”, povidone, silver, silver sulfate, betadine, and other antiseptics and proprietary and non-proprietary preservatives. By co-formulating preserved solutions for topical ophthalmic therapy and other topical ophthalmic drug products including suspensions and emulsions with multifunctional graft copolymers, corneal and ocular adverse events can be prevented, treated, mitigated or reduced. This is an important development because preserved drug products are generally easier to use and store and less expensive to manufacture. This invention reduces side effects associated with topical ophthalmic products that are preserved. The combination of preservatives or toxic APIs with a multifunctional graft copolymer in a formulation allows for more safer and more widespread use of preserved ophthalmic formulations. This benefit is particularly important when topical ophthalmic treatments with components that may lead to ocular adverse events are delivered chronically. Chronic use means greater than 4 weeks. Antibiotics, antivirals, anti-fungals, ant-infectives generally, anti-glaucoma medications, intraocular pressure lowering medications, neuroprotective agents, dry eye therapeutics, anti-inflammatory agents including but limited to topical steroids and nonsteroidal anti-inflammatory medications, ant-allergy medications with many specific names listed below can used chronically on the eye and often are prescribed that way. Prescription and over the counter medications can be used chronically.

Similarly, antibiotics and APIs directed at microorganisms can lead to ocular irritation or toxicity to corneal epithelial cells or conjunctival cells as a side effect. When PLL-g-PEG is formulated with antibiotics or antifungals or antimycotics or anti-acanthamoeba medications or antiviral medications, patients tolerate the PLL-g-PEG formulations better than products without, while efficacy is maintained either in prevention of infection or to treat a microbial infection. Patients show decreased rates of corneal epithelial toxicity in treated vs. controls in a clinical study, especially when frequent use is required to treat the offending pathogen or pathogens. The formulations are similarly effective. Preservatives and antimicrobials and APIs not yet discovered, but with some corneal toxicity at or below therapeutic dosing requirements can be utilized with multifunctional graft copolymers to enhance safety and optimize efficacy. Multifunctional graft copolymers can also support higher concentrations of APIs to confer a greater potency and maintain patient safety. Thus, formulating with, PLL-g-PEG, for example, allows for a wider therapeutic window, and greater efficacy. Poly-antimicrobials (meaning more than one active antibiotic) as well as single actives are included. Combination API drug products are included without limitation, but included for example are antibiotic/steroid combinations, antibiotic/antibiotic or more combinations, glaucoma medication/glaucoma medication combinations, dry eye and other API combinations, dry eye/dry eye combinations, and anti-inflammatory anti-allergy combinations. Formulations with other topical therapeutics including glaucoma therapies, allergy therapies, dry eye therapies, and anti-inflammatory therapies are included as potential formulations that are embodiments of the invention and help improve safety and maintain efficacy. For example, therapies directed inside the eye can safely traverse corneal and conjunctival epithelial cells with decreased toxicity enroute to their intraocular or deeper corneal targets (e.g. stroma or endothelium) as well.

Coformulating multifunctional graft copolymers for the mitigation of corneal toxicity or corneal nerve protection with other actives such as but not limited to antioxidants, free radical scavengers, anti-infectives, antibiotics, anti-fungals, anti-infectives; anti-helminthics, anti-parasitics, an anti-acanthamoeba agent, antiseptics, antivirals, steroids, NSAID's, vitamins, dry eye therapeutics, immunomodulators, neuroprotectants, cytoprotectants, glaucoma drugs, intraocular pressure lowering agents, PPAR agonists, PAR modulators, and other large or small molecules that have therapeutic ocular benefit (collectively in a wide range of eye diseases) are an embodiment of this invention. Formulating multifunctional graft copolymers with ophthalmic medications that may exhibit corneal surface or subbasal nerve toxicity when dosed chronically to help protect the ocular surface is an embodiment. In some embodiments these may be combination drug products. In other sequential delivery is a method of use.

In addition to formulating with an intent to improve the bioavailability, tissue kinetics, pharmacokinetics, pharmacodynamics, residence time, bioabsorption or bioadsorption, with other excipients and molecules or nanotechnology or electrical or iontophoretic methods, altering the polydisperity and the range and types or sizes of the multifunctional graft copolymers also accomplishes this important therapeutic goal. It is known that molecular weight can affect the penetration of molecules into the corneal and through the conjunctiva. Thus, more effective multifunctional graft copolymers, clinically in a human or other mammal, may be smaller in size in certain therapeutic or disease state situations. An embodiment is specifically using a smaller PLL based on chain length, or an average molecular weight MW. Smaller MW multifunctional graft copolymers to better penetrate into corneal epithelial tissue and to cross the conjunctiva is an embodiment of this invention. Reducing PLL chain length from a mean of 20 kiloDalton (kDa) to a mean of 15, 12, 10, 7, 5, 3, 2,1 kDa or 500 Daltons or 250 Daltons confers some efficacy benefit, either alone or in combination with related or widely disparate sizes. The combination of small and large MW multifunctional graft copolymers (wide variety of PLL-g-PEG structures for example) to optimize ADC tox reduction, to optimize corneal protection efficacy and corneal epithelial penetration and conjunctival penetration is also an embodiment of this invention. The size of the PEG side chain can likewise be altered to generate structures with smaller and larger MW sizes. PEG can be as large as 30 kDa or greater or down to 500 Daltons. Typical average PEG kDa sizes with efficacy range from 20 kDa to 0.4 kDa. 5 kDa, 2 kDa, and 0.5 kDa would be commonly used. The graft ratio may further be adjusted to enhance efficacy. For example, the graft ratio may be selected from 2, 3, 4, 5, 6, 7, 8, or 9 to improve efficacy.

Thus, for clinical use, various graft ratios, PLL sizes, and PEG sizes and chain lengths can be utilized in combinations to maximize benefit. Similarly, other cationic graft copolymers can be modified. An embodiment of this invention is to include multiple discrete sized and structured PLL-g-PEG (or mimetic cationic graft copolymers of multifunctional graft copolymers or dendrimers), to improve efficacy and safety. For example, but not limiting there may be included in a formulation 1% PLL 20 kDa MW, graft ratio 3.5, PEG 5 kDa MW structure and 1% PLL 10 kDa, graft ratio 5, PEG 2 kDa, and 1% PLL 5 kDa, graft ratio 6, PEG 0.5 kDa where structures are based on mean or average polymer MW sizes. Polydispersity for each mean may have a range of polydispersity indexes.

The effects on the cornea and conjunctiva can be identified with stains or vital dyes. For example, fluorescein, lissamine green, and/or rose Bengal can be utilized to assess for epithelial damage and can be used in clinical trials, claims, or to identify patients at risk and assess treatment efficacy. Outcome measures as reported by a patient can similarly be used to assess patients' response to therapy and to identify those who may benefit from treatments described herein; in particular, the Symptom Assessment iN Dry Eye” (SANDE) questionnaire utilizes a 100 mm horizontal VAS technique to quantify patient symptoms of ocular dryness and/or irritation. The Ocular Surface Disease Index (OSDI) scale can also be used. Other scales measuring patient comfort and symptoms can be used. Technology including but not limited to anterior segment optical coherence tomography including epithelial mapping, in vivo confocal microscopy (evaluating the basal layers, sub basal plexus, and more superficial layers of the corneal epithelium), and anterior segment photography can assess the anatomic and microscopic status of the cornea and surface status depending on which applicable technology is used. Refractive changes and visual acuity testing can also be helpful in these settings. Tear film break up time can similarly be utilized. Thus, the above approaches are examples, but in no way limiting, regarding methods to assess the eye in a clinical setting.

Included herein are also methods for inhibiting, reducing, or preventing viral (e.g. SARS-COV-2) infectivity and reducing the severity of the disease course can be accomplished in a subject via topical or local administration of a formulation comprising a graft co-polymer having a positively charged moiety and a hydrophilic moiety or a block co-polymer having a positively charged moiety and a hydrophilic moiety to a biological surface of a subject. Because charge dynamics are complex, negative charge-based passivation methodology can also be identified, and these discoveries are also considered and utilized herein. Formulations with the polymers claimed herein can be administered topically or locally in accordance with methods of the present invention to biological surfaces of a subject including, but not limited to, skin, mucous membranes, oral mucosa, nasal mucosa, and the surface of the eye. By “subject”, as used herein it is meant to be inclusive of all animals and in particular mammals such as, but not limited to, humans and dogs as well as agricultural animals such as bovine, ovine, and porcine.

Graft co-polymers used in the methods and formulations of the present invention are polymers having a linear section of repeat units called the “backbone”, with at least one side chain of repeat units (called a “graft”), usually of a different chemistry, branching from a point along the backbone. In one embodiment, the graft co-polymer comprises a cationic backbone and side chains that are water soluble and non-ionic. In another embodiment, the graft copolymer comprises a water-soluble non-ionic backbone and cationic side chains. In another embodiment, negative or anionic graft copolymers are utilized.

Block co-polymers used in the methods and formulations of the present invention are polymers in which linear sections of a first section of repeat units are connected end-to-end with linear sections of subsequent repeat units that are chemically dissimilar to the first.

Formulations for use in the methods of the present invention comprise a block or graft co-polymer (a multifunctional copolymer) having one section, either the backbone, the graft or the block, that adheres to a biological surface tissue such as, but not limited to, cells, epithelial cells, mucosa, mammalian tissue, respiratory cells, alveoli, tracheal and bronchial tissue, nasal mucosa, oral mucosa, and the eye surface including corneal and conjunctival epithelial cells as well as limbal stem cells, limbal cells, limbal epithelial stem cell, basal stem cells, basal cells, early transient amplifying cells, transient amplifying cells, and corneal epithelial cells generally, as well as the glycocalyx and microvilli associated with any such cells. In addition to electrostatic forces bioadhesion, another, chemically different section, either the backbone, the graft, or block, is hydrophilic and induces a passivation and reduction of interaction between viruses in one embodiment and ADCs in another. Interactions are effectively diminished for benefits to human health. Viral transmission and contagious diseases transmissions can be reduced, especially those for which there is insufficient host or herd immunity to prevent an epidemic or pandemic. One embodiment is a method for decreasing novel viral infectivity for which humans have not developed herd immunity to help mitigate disease and the severity of epidemics and pandemics.

Human health is improved by reducing ADC toxicity in general, and to the eye and cornea specifically. Specifically, the health of the corneal epithelium is promoted. Patients can better tolerate ADCs for the treatment of malignancy or other conditions while maintaining healthier corneal epithelium resulting in fewer and less severe reductions in eyesight/visual acuity and fewer and less symptomatic eyes. As described herein, the term patient and subject may be used interchangeably. The tissue-adhesive sections of a graft or block co-polymer in the formulations used in the methods of the present invention may be cationic, in which case the polymer adheres to the biological surface by electrostatic attraction. The interaction may be anionic or hydrophobic using hydrophobic moieties in combinations herein as well—hydrophobic interactions, as well as anionic interactions hold promise.

Examples of cationic polymer sections of graft or block co-polymers of formulations useful in the methods of the present invention include, but are not limited to: poly(L-lysine) (PLL), poly(2-vinyl pyridine) and poly(4-vinyl pyridine) and vinyl co-polymers containing those repeat units, and poly(aminocthyl methacrylate) homo—and co-polymers containing N,N dimethylaminoethylmethacrylate) repeat units. Another cationic polymer section which can be used is chitosan (a co-polymer of glucosamine and N-acetyl glucosamine where 5-100% of the repeat units are glucosamine) and synthetic derivatives thereof. Examples of hydrophobic polymer sections of graft or block co-polymers of formulations useful in the methods of the present invention include, but are not limited to, long-chain aliphatic hydrocarbons, polyethylene, poly(propylene oxide), polystyrene, poly(methylmethacrylate), poly(butylenes oxide), and the like. The hydrophilic section of the polymer may be non-ionic if the tissue adhering section is cationic, or anionic if the tissue-adhering section is non-ionic (and hydrophobic).

Examples of anionic polymer sections of graft or block co-polymers of formulations useful in the methods of the present invention include, but are not limited to: polyacrylic acid (PAA), polymethacrylic acid, poly(sodium styrene sulfonate), carboxylated cellulosics such as carboxymethylcellulose (CMC), poly(itaconic acid), poly(maleic acid), poly(aspartic acid), poly(glutamic acid), polyphosphates, polynucleic acids, poly(acrylamidopropanesulfonic acid), anionic natural gums, anionic carbohydrates, carageenan, alginates and hyaluronic acid.

Examples of non-ionic hydrophilic polymer sections of formulations useful in methods of the present invention include, but are not limited to, poly(ethyleneglycol) (PEG), poly(vinylalcohol), polysarcosine, poly(vinylpyrrolidinone), and the like. Examples of anionic hydrophilic polymer sections include homopolymers and co-polymers containing, for example, acrylic acid, methacrylic acid, itaconic acid, maleic acid, styrene sulfonic acid, carboxymethyl cellulose, carboxyethylcellulose, succinylated chitosan, cellulose sulfate, and the like.

By block or graft co-polymers it is meant to describe the architecture of the polymer.

By “mimetic polymers” it is meant an alternative chemical/molecular approach to creating the same behavior and attributes of an effective and tested polymer. Multiple mimetics exist as, for example, PLL-g-PEG is effective. The effect is primarily drawn from the macromolecular passivation.

By “passivation” it is meant that a cell or biological surface has a reduced interaction capability with a virus or antibody drug conjugate such that infection or macropinocytosis, cell internalization of the virus or ADC, is reduced. The phenomenon is steric interference and reduces an ability of charge interactions to play a role in the virus or ADC interaction. By “passivation” it is also meant that a virus or ADC surface has a reduced interaction capability with a cell at risk such that infection or macropinocytosis, cell internalization of the virus or ADC, is reduced.

Passivation may occur on the ADC, cytotoxic drug, or virus. Passivation may also take place by interactions on the cells to make them less susceptible to ADC, drug, or virus uptake.

Graft co-polymers may have a cationic (or non-ionic hydrophobic or anionic) backbone made from a polymer chosen from the list, supra, and hydrophilic grafts, or have a hydrophilic backbone, and cationic (or non-ionic hydrophobic) grafts chosen from the list, supra. For graft co-polymers, grafts may arise from every repeat unit in the backbone or may be intermittently spaced along the backbone (with uniform or random frequency). For example, a useful polymer in formulations for use in the methods of the present invention is PLL-g-PEG where the backbone is the cationic polymer poly(L-lysine) and the grafts are made from the hydrophilic polymer poly(ethylene glycol). The PLL backbone may be from 3 repeat units to several thousand repeat units long, and the PEG grafts may be from 1 to several thousand repeat units long. The PEG grafts may be attached to every PLL repeat unit, every other PLL repeat unit, every third repeat unit or less frequent. In one embodiment, there is a PEG graft on average at every third PLL repeat unit. Similar characteristics may be applied to mimetic polymers.

Block co-polymers comprising at least one block that is cationic and at least one block that is water soluble and non-ionic are also useful in formulations for use in methods of the present invention. In one embodiment, the block co-polymer comprises at least one block which is hydrophobic and at least one block which is water soluble and anionic, cationic or non-ionic. Anionic blocks are considered.

Examples of water soluble non-ionic co-polymer blocks include, but are not limited to, poly(ethylene glycol) (PEG), polyvinyl alcohol (PVA), poly(hydroxyethyl methacrylate) (pHEMA), poly(acrylamide), poly(vinyl pyrrolidone) (PVP), poly(ethyl oxazoline) (PEOX), polysarcosine, polysaccharides, and copolymers of any two or more thereof.

Examples of water soluble anionic co-polymer blocks or backbones include, but are not limited to, polyacrylic acid (PAA), polymethacrylic acid, poly(sodium styrene sulfonate), carboxylated cellulosics such as carboxymethylcellulose (CMC), poly(itaconic acid), poly(maleic acid), poly(acrylamidopropanesulfonic acid), anionic natural gums, anionic carbohydrates, carageenan, alginates and hyaluronic acid.

Examples of water soluble cationic co-polymer blocks include, but are not limited to, polymers based on vinyl pyridine, N,N-dimethylaminoethylacrylate, N,N-dimethylaminoethylmethacrylate, allyl tri(alkyl) ammonium halides, poly(amino styrene), chitosan, polyethyleneimine, polyallylamine, polyetheramine, polyvinylpyridine, polysaccharides having a positively charged functionality thereon, polyamino acids such as, but not limited to, poly-L-histidine, poly-ε-benzyl-L-histidine, poly-D-lysine, poly-DL-lysine, poly-L-lysine, poly-ε-CBZ-D-lysine, poly-CBZ-DL-lysine, poly-ε-CBZ-L-lysine, poly-DL-ornithine, poly-L-ornithine, poly-Δ-CBZ-DL-ornithine, poly-L-arginine, poly-DL-alanine-poly-L-lysine, poly(-L-histidine, L-glutamic acid)-poly-DL-alanine-poly-L-lysine, poly(L-phenylalanine, L-glutamic acid)-poly-DL-alanine-poly-L-lysine, and poly(L-tyrosine, L-glutamic acid)-poly-DL-alanine-poly-L-lysine, copolymers of L-arginine with tryptophan, tyrosine, or serine, copolymers of D-glutamic acid with D-lysine, copolymers of L-glutamic acid with lysine, ornithine, or mixtures of lysine and ornithine, and poly(L-glutamic acid).

Examples of hydrophobic co-polymer blocks include, but are not limited to, alkanes, alkenes, alkynes, poly(isobutylene), polyesters such as poly(caprolactone) (PCL), poly(lactic acid) (PLA), poly(glycolic acid) (PGA), and copolymers therefrom (PLGA), polyamides such as nylon(6,6) and Nylon(12), polyurethanes, poly(propylene oxide), poly(tetramethylene oxide), polyethylene, polypropylene, polystyrene, poly(acrylates) such as polymethyl acrylate (PMA), poly(methacrylates) such as poly(methylmethacrylate) (PMMA), poly(sulfones), poly(etheretherketones) (PEEKs), poly(phosphazines), poly(carbonates), poly(acetals) and poly(siloxanes).

If in the descriptions above and herein, one molecular entity can apply to another paragraph, but was omitted, it can be considered where applicable so included. Similarly, terms can be moved about where indicated to support eventual claims.

As will be understood by the skilled artisan upon reading this disclosure, graft and block as well as triblock and dendrimers are contemplated, and these configurations can also be used as embodiments of the current invention.

An exemplary block co-polymer comprising a triblock configuration is PLURONIC® F127, also referred to as Poloxamer 407, containing a poly(ethylene oxide) hydrophilic block (“PEO”), a poly(propylene oxide) hydrophobic block (“PPO”) and another PEO block. Other block co-polymers for use in the present invention may contain only one hydrophilic block and one hydrophobic block, or may contain several alternating blocks, for example the PPO-PEO-PPO block co-polymers (PLURONIC®, block co-polymers based on ethylene oxide and propylene oxide, BASF, Florham Park, NJ). Additional exemplary PLURONIC block co-polymers useful in the present invention include, but are not limited to, PLURONIC 10R5, PLURONIC 17R2, PLURONIC 17R4, PLURONIC 25R2, PLURONIC 25R4, PLURONIC 31R1, PLURONIC F 108 Cast Solid Surfacta, PLURONIC F 108 Pastille, PLURONIC F 108 Prill, PLURONIC F 108NF Prill Polaxamer 338, PLURONIC F 127 Prill, PLURONIC F 127 NF, PLURONIC F 127 NF 500 BHT Prill, PLURONIC F 127 NF Prill Poloxamer 407, PLURONIC F 38, PLURONIC F 38 Pastille, PLURONIC F 68, PLURONIC F 68 Pastille, PLURONIC F 68 LF Pastille, PLURONIC F 68 NF Prill Poloxamer 188, PLURONIC F 68 Prill, PLURONIC F 77, PLURONIC F 77 Micropastille, PLURONIC F 87, PLURONIC F 87 NF Prill Poloxamer 237, PLURONIC F 87 Prill, PLURONIC F 88 Pastille, PLURONIC F 88 Prill, PLURONIC F 98, PLURONIC F 98 Prill, PLURONIC L 10, PLURONIC L 101, PLURONIC L 121, PLURONIC L 31, PLURONIC L 35, PLURONIC L 43, PFLURONIC L 44, PLURONIC L 44 NF Polaxamer 124, PLURONIC L 61, PLURONIC L 62, PLURONIC L 62 LF, PLURONIC L 62D, PLURONIC L 64, PLURONIC L 81, PLURONIC L 92, PLURONIC L44 NF INH surfactant Polaxamer 124, PLURONIC N 3, PLURONIC P 103, PLURONIC P 104, PLURONIC P 105, PLURONIC P 123 Surfactant, PLURONIC P 65, PLURONIC P 84, and PLURONIC P85. Where applicable, all particle sizes of the block co-polymers are included, for example PLURONIC F127 and PLURONIC F87 are available as prill and microprill products. Non-ionic surfactants, for example, containing a hydrophobic segment and a PEO block are considered here as block co-polymers.

Additional exemplary block or graft co-polymers which can be used in the present invention are disclosed in U.S. Pat. Nos. 5,578,442 and 5,834,556, teachings of each are herein incorporated by reference in their entirety.

Dendrimers can confer similar properties to the multifunctional graft copolymers and can be considered as potential solutions to the ophthalmic adverse event profiles considered herein. The non-multifunctional graft copolymers identified herein may independently also have characteristics conferring some benefit in topical ophthalmic formulations to mitigate ADC toxicity.

The block or graft co-polymers are included in formulations for use in the methods of the present invention at concentrations ranging between 0.001% and 40%, more typically 0.01% to 25%, on a weight/weight basis as a component of the formulation. In certain preferred embodiments, the concentration of formulation is between 1% and 3% graft co-polymer on a weight/weight basis. The formulation is, in some embodiments, a powder, such as a lyophilized powder for delivery to tissue or for reformulation and dissolution, the powder may be from 0.001% to 100% block or graft copolymer. Copolymers may be water soluble in some embodiments.

Combinations of the copolymers are considered and included herein whereas the percentage of the formulation may apply to each different component. The use of higher concentrations is a method to increase penetration of the multifunctional graft copolymers deeper into the cornea (int the ˜50 μm thick epithelium—or greater or less depending on the patient variabilities) and across the conjunctiva, especially at the limbus to reach the limbal stem cells and progenitor cells. Higher concentrations also lead to an even greater longevity of effect. Greater concentrations are defined here as between 3% and 25% (weight/weight), but up to 40%. An exemplary greater concentration formulation is 5% PLL-g-PEG or 10% PLL-g-PEG. Multifunctional graft copolymers generally can be likewise formulated for greater tissue penetration and longer duration of the multifunctional graft copolymer. Similar concentrations can be considered for greater concentrations as per above.

In the Working Examples described herein in humans in which the formulation is described at present, when delivered as solutions or suspensions, the amounts of co-polymers are between about 0.1% and 5%. In addition, the amount of co-polymer can be, 0.01% to 3%, 0.1% to 2.5%, or 0.5 to 4%.

Additional exemplary components which can also be incorporated into pharmaceutical formulations and coatings for use in the present invention include, but are not limited to, PLURONIC gelling agents such as, but not limited to F127, F108 as well as additional PLURONIC agents listed supra. Furthermore, in one embodiment, these components are used at fractions below that required for gelling activity.

Other components (either active or inactive ingredients) which can be included in these pharmaceutical formulations include, but are not limited to, lipids, oils, surfactants, water, lubricating polymers, typical surfactants, buffers, salts, physiologic ions, proteins, topical emollients, excipients typically used in oral, topical, mucosal, dermatologic and ophthalmic formulations, lubricants such as PEG 400, carboxymethylcellose, hydroxypropyl methylcellulose, mineral oil, propylene glycol, glycerin, hypromellose, white petrolatum, polyvinyl alcohol, liposomes, mannitol, hydroxypropyl guar, dextran 70, viscoelastics, and hyaluronic acid, as well as combinations thereof. Additional ingredients may include those routinely included in mouthwashes, nasal sprays, shampoos, soaps, and conditioners. Such components may be included in the formulations in varying percentages ranging from less than 0.1% to 99% w/w %, more preferably less than 1% to 10% w/w %. Other components which can be included in these pharmaceutical formulations include, but are not limited to preservatives such as Polixetonium, polyquaternium-42, Polyquaternium-1, Polyquat, Alkyl-hydroxy benzoate preservatives, parabens, hydrogen peroxide, benzalkonium chloride (BAK), cetylpyidimine chloride, cetalkonium chloride, sodium perborate, Purite, disappearing preservatives, Polyhexamethylene biguanide (PHMB), chlorobutanol, Benzododecinium bromide, “Ionic buffered system”, povidone, silver, silver sulfate, betadine, and other antiseptics and proprietary and non-proprietary preservatives. Also, PLL-g-PEG, may act as a preservative. Antibiotics, antiviral agents (whether small molecule or biologics) may be contained in the formulation. In some embodiments, preservative free formulations are preferred.

Further, in some embodiments, the formulations and coatings may include one or more additional active pharmaceutical ingredients. Examples include, but are in no way limited to anesthetics, antibiotics, antivirals, anti-inflammatory agents, intraocular pressure lowering agents, artificial tears, lubricating products, dilating agents, immunosuppressives, antiangiogenic agents, monoclonal antibodies, proteins, peptides, neuroprotectants, small molecules and antibodies. In some embodiments, the formulation is delivered prior to personal protective equipment use.

Some exemplary, without limitations, additional medications that can be included in these formulations include, as indicated for human benefit: antiviral agents including remdesivir, anti-retroviral agents, rimantadine and others selected from this list: Abacavir Use for HIV, Acyclovir (Aciclovir) Use for herpes e.g. Chicken pox, Adefovir Use for chronic Hepatitis B, Amantadine Use for influenza, Ampligen, Amprenavir (Agenerase) Use for inhibition of HIV, Arbidol, Atazanavir, Atripla (fixed dose drug), Balavir, Baloxavir marboxil (Xofluza), Biktarvy, Boceprevir (Victrelis), Cidofovir, Cobicistat (Tybost), Combivir (fixed dose drug), Daclatasvir (Daklinza), Darunavir, Delavirdine, Descovy, Didanosine, Docosanol, Dolutegravir, Doravirine (Pifeltro), Ecoliever, Edoxudine, Efavirenz, Elvitegravir, Emtricitabine, Enfuvirtide, Entecavir, Etravirine (Intelence), Famciclovir, Fixed dose combination (antiretroviral), Fomivirsen, Fosamprenavir, Foscarnet, Fosfonet, Fusion inhibitor, Ganciclovir (Cytovene), Ibacitabine, Ibalizumab (Trogarzo), Idoxuridine, Imiquimod, Imunovir, Indinavir, Inosine, Integrase inhibitor, Interferon type I, Interferon type II, Interferon type III, Interferon, Lamivudine, Letermovir (Prevymis), Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Peginterferon alfa-2b, Penciclovir, Peramivir (Rapivab), Pleconaril, Podophyllotoxin, Protease inhibitor (pharmacology), Pyramidine, Raltegravir, Remdesivir, Reverse transcriptase inhibitor, Ribavirin, Rilpivirine (Edurant), Rimantadine, Ritonavir, Saquinavir, Simeprevir (Olysio), Sofosbuvir, Stavudine, Synergistic enhancer (antiretroviral), Telaprevir, Telbivudine (Tyzeka), Tenofovir alafenamide, Tenofovir disoproxil, Tenofovir, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), Zidovudine. Hydroxychloroquine, chloroquine, and azithromycin may be included in said formulations. Biomolecules selected from antibody products with poly or monoclonal, biomolecules that bind to antibodies, proteins, antigens are included in potential formulations. Fragments of antibodies, trap molecules, and biomolecules that bind to ADCs and other antibodies, cell receptors and proteins relevant to conditions addressed in this application are included, whether known, in development, or not yet developed or conceived. The key is a combination of the claimed copolymers formulated with another effective agent in management of the disease addressed to improve performance or efficacy-including synergistic therapies. Biomolecules are produced by a living organism. Synthetically produced antibodies, recombinant proteins, and other large and small molecules for which new techniques lead to new nomenclatures are also included. ACE2 receptor blockers, steroids (glucocorticoid, androgens, estrogens, etc.) are also potential formulations components, and are considered within various embodiments of the current invention. Therapeutic and targeted growth factors applied topically including but not limited to hepatocyte growth factor and human recombinant dHGF (5-amino acid deleted hepatocyte growth factor) are contemplated in various embodiments.

pH of formulations of the present invention is in a physiologic range depending upon the site of administration and the site of the biological surface or membrane that is to be modified. Typically, the pH is above 3, e.g., above 5.6 and below 9. In preferred embodiments, the pH of the formulation is 5.5 to 8.5.

Formulations may include new or established dry eye or corneal therapeutics including, but not limited to, cyclosporine, lifitigrast, LFA-antagonists, steroids including loteprednol, dexamethasone, flucinolone, difluprednate, aldehyde traps, fonaldepar, varenicline, visomitin, kinases, silk derived proteins, voclosporin, omega 3 fatty acids, and any demulcents or lubricants in the OTC monograph referenced completely herein. Importantly, some embodiments of the formulations have very low viscosity (lower than most artificial tear products on the market). This advantage is that vision is not blurred on instillation, sprays work easily through small diameter nozzles, and mouthwashes rinse out completely and easily and are well tolerated. Electrospray and other microelectronic and mechanical delivery systems, high precision multi-jet systems and other approaches to microdose release are included as possible delivery systems, among others. The copolymers reside following rising. The residual bioadhesive polymers of course already bind to the cells for which they protect. For example, the formulation in one test had a viscosity of 2.7 cP. Furthermore, the benefit conferred with these inventions demonstrates the value of the graft or block copolymer as the mechanism of effect.

As described in Example 1, In Vitro experiments, this approach is effective in reducing viral infectivity. In other examples, reduced ADC entry into epithelial cells and decreasing eye adverse event are shown.

Furthermore, as described in Examples herein, performance of these block and graft copolymer formulations is assessed in the eyes of human volunteers. Previous experiments have shown the eye drop was well tolerated. For example, of the multiple initial human exposures, there was no irritation or discomfort reported by any subject. Additionally, there were no reports of blur following single and repeated instillation of 50 microliters or less. Thus, a product with lower viscosity is an embodiment of this invention.

As will be understood by the skilled artisan upon reading this disclosure, however, alternative ophthalmic delivery means including, but not limited to, intraocular, periocular, conjunctival, subconjunctival, transconjunctival, peribulbar, retrobulbar, subtenons, transscleral, topical gel, topical dispersion, intraorbital, intrascleral, intravitreal, subretinal, transretinal, choroidal, uveal, intracameral, transcorneal, intracorneal, intralenticular (including phakia and psuedophakia), and in or adjacent to the optic nerve, can be used. The invention can be used with polymeric, and other delayed release formulations for prolonged drug delivery. The invention can be used with depot formulations. The invention can be intravenous to treat other ADC toxicities such as thrombocytopenia.

Of importance in the current invention for use in ADC toxicity and also for viral infection, there are in vitro and in vivo opportunities of commercial value and product development value. For example, since these copolymers with bioadhesive and passivation moieties are shown effective in these conditions both in vitro and in humans, their use in development has value. A cell may be treated with a copolymer formulation, and then ADCs can be added. Tissue culture can also be used including corneal epithelial models. Those that show less uptake in the setting of copolymer presence may be selected for development as there is a known ameliorating approach to off-target or macropinocytotic uptake. The models may use epithelial cell lines, harvested epithelial cells, megakaryocytes, other cells, and human umbilical vein endothelial cells. Cell types with risk for toxicity are used in some laboratory development procedures. Likewise, antiviral efficacy in treatments and preventive measures may be assessed in the laboratory/developmental setting of viral and copolymer exposures to select synergistic and optimal molecules and formulations for human development. Thus, a claim to treating a cell broadly is a pertinent and useful invention to advance therapies for human health addressed herein.

COVID 19, the infection caused by SARS-Cov-2 is a devastating disease for many patients with a death rate up to 3%, and many more infecting requiring hospitalization and ICU care. Millions of patients have been infected and death rates are continually increasing into the hundreds of thousands. More than 3 million global patients have been infected and over 200,000 deaths within a six-month period have occurred. Patients of older age and with pre-existing medical conditions are at higher risk for morbidity and mortality. High viral load exposures are also a significant risk for morbidity. SARS-Cov-2 has caused a global pandemic, and better, and alternative treatments are needed. New pandemics may also develop, and this approach is not specific to one viral strain, although the spike protein in SARS-Cov-2 is amenable to passivation with the graft copolymer and block copolymer approach.

“Viral infectivity” relates to the exposure of at-risk host cells to a pathogenic virus. Using protein receptor and other uptake mechanisms viral particles can enter a cell, release their RNA or DNA and hijack the cells protein manufacturing and nucleotide machinery or other metabolic processes to manufacture more viral particles which are then released to infect other cells and other organisms. Without being limited to any particular mechanism, a formulation comprising a graft copolymer as described above (cationic, hydrophobic, anionic, with hydrophilic side chains), such as PLL-g-PEG, adheres to the biological surfaces of viral particles (including the spike for SARS-Cov-2) or other cell entry mediating proteins on the lipid membrane shell of the virus, or on the viral particle generally. The charged, hydrophobic, or anionic moiety, PLL for example, moiety leads to the bioadhesion. The hydrophilic (e.g. PEG) moiety prevents and/or reduces the interaction with the target cell. When applied as a drop, spray mist, or rinse, the graft co-polymer is able to move from surface tissue to viral particle due to physical chemistry on-off bonding associated with electrostatic interactions. Passivation moieties can be non-ionic, nonionic, and inert, as well as hydrophilic. The graft copolymer also protects the cells at risk in a similar manner and directly interferes with receptor proteins in some cases (e.g. ACE2) to enhance activity. The combined exposures to viral particles and at-risk cells reduce substantially their interaction and viral infectivity. Decreased interaction is beneficial to cells, tissues, and organisms including humans by decreasing pathogen or toxin exposure, thereby decreasing morbidity and mortality associated with these agents. For example, lower viral loads can decrease the severity of subsequent infection and allow the host defenses better opportunity to work. Decreasing viral exposure reduces transmission rates and possibly the severity of viral infection. Accordingly, the method of reducing SARS-Cov-2 with infection as described herein will be an important additional approach to safely protect a subject.

Similar formulations have already been used in the eye with no adverse events, and are safe for oral, respiratory, and nasal passageway use. PLL-g-PEG is made from an amino acid and PEG. Extensive reviews show the formulations are safe for use in humans and animals.

These formulations can also be widely used in accordance with the present invention to reduce transmission of SARS-Cov-2 and novel viruses via nasal and inhalational applications in settings including, but in no way limited to, hospitals, emergency departments, intensive care units, airplanes, preschools and schools, homes of affected viral individuals, as well as nursing homes and chronic care facilities. Heath care workers, and first responders may also benefit. “Novel” or “new” viruses means those with mutations or general characteristics that demonstrate humans, generally—the majority of the population, does not have resistance through prior exposure or vaccines. “Transfection” means how the virus enters the host. “Epidemics” and “pandemics” are determinations made often by health authorities. Pandemic is disease prevalent over a country or the world at a particular time. Epidemic is a widespread occurrence of an infectious disease in a community at a particular time. The invention particularly relates to viral pandemic and COVID19. COVID19 is the viral illness caused by SARS-Cov-2. Herd immunity is the resistance to the spread of a contagious disease within a population that results if a sufficiently high proportion of individuals are immune to the disease, through vaccination or previous exposure to the virus resulting in the production of antibodies. Herd immunity requires resistance to a virus in significant numbers. Important utilities of the formulations exist for children and adults with infirmities, such as immunodeficiencies, chronic disease and other chronic conditions such as cystic fibrosis, which leave the host more susceptible to routine illness.

Corneal epithelial microcyst like epitheliopathy is recently identified problem known to be associated with and/or due to ADC treatment, and it has recently become important clinically as ADCs with corneal epithelial toxicity are entering commercial use after many years of development. Furthermore, the ADC effects on the cornea may include changes in the thickness of the epithelium (thinning and/or thickening or both) and epithelial thickness irregularities. There may be refractive error changes associated with these changes and due to ADC toxicity. These findings are included in the spectrum of ADC corneal toxicity for which a therapeutic intervention, prevention, treatment, therapy, or mitigation strategy is included herein. Patients can develop irregular astigmatism and other ocular aberrations associated with ADC therapy. Spectacle changes can help but are inconvenient and do not treat the underlying condition. ADCs are under development as an effective therapeutic for many forms of oncologic disease. These are costly and resource demanding development programs, and the need to limit use due to ophthalmic toxicity is significant. At times human toxicity isn't manifest until clinical trials. It is important to allow patients to maintain ADC therapy for optimal oncologic (or other indicated) disease outcomes (survival or progression free responses, beneficial effect) and corneal epithelial toxicity is an adverse event that can limit therapy or even cause patients to discontinue ADC-related therapy. Significant ophthalmic and other types of adverse events associated with ADCs are a problem for patients and treating physicians. The payload or warhead is thought to cause the adverse events (ophthalmic or in some embodiments otherwise such as other cells or cells in culture or tissue culture) following its release (cleavage of the linker) from the ADC once in the cell.

Without being bound to a particular theory or mechanism of toxicity, but as a method to explain the presumed utility of this invention for which the discovery of benefit is demonstrated in vitro and in vivo.

ADCs are able to gain exposure to the eye through one of two ways based on the toxicity seen clinically. First, the ADC may get to limbal stem cell daughter cells and eventually basal stem cells and basal epithelial cells via release from the perilimbal circulation (including but not limited to the palisades of Vogt which have a distinct vasculature with narrow, barely visible, arterial and venous components of radially oriented hairpin loops) into the extracellular space and subsequent non-specific uptake by basal cells that then move into the cornea.

“Macropinocytosis” has been described as a mechanism for ADCs to enter cells such as the corneal cells affected by the toxicity as they typically lack the specific receptor that the antibody portion of the ADC uses to enter the cell. Thus, the entry of the ADC into the cell is nonspecific and can be considered off target. Embodiments of the inventions herein ameliorate or reduce off target ADC-induced corneal cell toxicity including limbal stem cells, daughter cells, transient amplifying cells, wing cells, basal cells, corneal epithelial cells, and terminal epithelia differentiated cells. ADCs or toxic biologics can also enter cells through other forms of pinocytosis. Cell surface stereochemistry can also confer benefit when multifunctional graft copolymers are used in this setting.

The ADC may also gain exposure to the cornea via the tears. Drugs have been reported to be secreted in tears. Cytokines of many kinds have been identified in the tears where the secretion from the lacrimal system is suggested path to their presence. The tear film contains hundreds of proteins and/or enzymes, secretion by lacrimal glands again is the understood mechanism. The leakage from the plasma either across the blood/tear barrier or by leakage from tissue interstitial fluid has also been described as paths to protein presence in tears. Antibodies are found in tears. Multifunctional graft copolymers may coat, bind, or otherwise interfere with ADCs and biologics secreted in tears to confer benefit.

The superficial corneal epithelial cells have no blood supply and must obtain fluid replenishment or nourishment in some manner independent from a direct blood supply. Epithelial cells use macropinocytosis as a method of nourishment. The plasma membranes of cells contain combinations of glycosphingolipids, cholesterol and protein receptors organized in glycolipoprotein lipid microdomains termed lipid rafts. Lipid-raft internalization is a process documented in corneal epithelial cells for the internalization of extracellular material. Macropinocytosis is a type of endocytosis whereby extracellular material is captured by a cell. (Macropinocytosis is a means by which eukaryotic cells ingest extracellular liquid and dissolved molecules. Pinocytosis may be used as a term herein in embodiments as well. Pinocytosis is the ingestion of liquid into a cell by the budding of small vesicles from the cell membrane. Micropinocytosis is considered herein and may be used as term in embodiments as well and can be used in any specified embodiment where either macropinocytosis or pinocytosis generally are used. Micropinocytosis is the incorporation of macromolecules or other chemical substances into cells by membrane invagination and the pinching off of relatively minute vesicles.) Toxins and pathogens utilize endocytosis and macropinocytosis to gain entry into many cell types. ADCs can get into cells by macropinocytosis.

Macropinocytosis occurs in many types of cells. It has been shown that macropinocytosis is a method for ADC entry into corneal epithelial cells. These methods of cellular uptake of extracellular components are natural; there inhibition in select settings is, however, beneficial. Macropinocytosis is a likely method for superficial epithelial cells internalizing ADCs. Being the least specific pathway, it is directed by actin-driven membrane protrusions which create large endocytic vesicles known as macropinosomes. Eventually, those vesicles fuse with lysosomes. Once an ADC is within a lysosome, the payload, specifically in the setting of ADV tox, maytansinoid and auristatins are associated with toxicity. However, tubulin inhibiters generally are potentially causative of corneal toxicity. The exposure to the cells most adversely affected by the toxic payload generally, or tubulin inhibitors specifically are cells that either divide (mitotic activity that is microtubule dependent) or migration (also microtubule involvement.

Payloads are the cytotoxic molecule that is linked to an antibody which is subsequently cleaved at the linker by intracellular enzymes. It is the method by which ADCs deliver a toxin to target (neoplastic) cells, but other cells are also adversely affected, and the ADCs can gain entry even without expressing an ADC interacting receptor/protein. Pinocytosis is a means ADCs enter cells. Other payloads besides tubulin inhibitors may show corneal epithelial toxicity, and those alternatives cytotoxic payloads are considered herein. Once the payload is cleaved from the ADC, it actively performs its chemical function such as the inhibition of polymerization of tubulin into microtubules which are needed for cell division, and in some cases cell migration. The presence of tubulin inhibitors in a dividing cell can cause cell death (followed by apoptosis and pyknosis). Apoptosis and pyknosis have been observed in corneal epithelial cells in ADC toxicity. The pyknotic cells are presumed to be the cause of the examination finding of microcyst like keratopathy. The dead or dying, and dysfunctional cells are seen in the epithelial layer on slit lamp examination. The extent can be quantified.

It is possible that once cleaved in a cell that uptakes the ADC, that the cytotoxic molecule may be released from said cell and cause a bystander effect. Thus, reducing cellular uptake and cleavage may be an intervention that has an excess relative reduction (greater number of cells may be protected than actually-uptake the ADC into one cell-due to bystander death). Bystander death means release of the cytotoxin, once cleaved into the extracellular space where it then enters subsequent cells. It thus may move through the corneal epithelial layers once engulfed by a more superficial cell. It also may be released at the limbus once taken up by a stem cell and released into other nearby stem cells and diffuse inward into basal stem cells. Such diffusion of the cytotoxic agent may be reduced with the methods of this invention. Not only may the approach reduce uptake and cleavage, but polymer may limit the movement and re-uptake of local cytotoxin. Regardless of the mechanism, the effect is real and valuable.

Toxicity in the human is or may be in certain cases confined to the epithelial layers (not involving the stroma or endothelium). Without wishing to be bound by any particular theory, the stroma or endothelium may be involved secondarily or in some ADC drug products. Normal replenishment of superficial epithelial cells is interfered with and the superficial epithelial layer becomes abnormal and can manifest with punctate staining, corneal epithelial defects, and abnormal refracting surfaces. Visual acuity is adversely affected in many cases. The patient may develop symptoms such as blurred vision, dry eye, corneal foreign body sensation, ocular discomfort, ocular irritation. Corneal infection, stromal keratitis, and ulcerative keratitis have been reported with ADC therapy. These are all significant adverse events and dose holds and dose delays are not infrequently required due to these toxicities. Histopathologic examination in cases of cytarabine (a similar situation to cases encountered with some ADCs) eye shows profound degeneration of the rapidly dividing basal epithelial cells, which leads to formation of epithelial microcysts.

Ocular irritation is an ADC related toxicity or adverse event, but not the only symptom. Corneal superficial punctate epitheliopathy, corneal erosions and epithelial defects, corneal ulcers, corneal infections, corneal perforations, can or potentially can occur from ADC induced corneal toxicity. Foreign body sensation and reduced visual acuity can occur. Dose holds, dose delays, and dose reductions for this life saving treatment can be required. Patients need vision for driving and reading. Vision is a high driver of quality of life. Allowing patients to maintain a more regular dosing schedule as well as minimizing eye symptoms is a benefit of this invention. Patients will have fewer and less severe adverse events leading to better outcomes from less uninterrupted ADC.

ADC related corneal toxicity has no treatment directed at the mechanism as demonstrated herein. Symptomatic management is the extent of the interventions. Commercially available lubricant eye drops, punctual occlusion, bandage contact lenses help with symptoms.

In one aspect, a method to decease the adverse events associated with antibody-drug conjugate usage in the setting of an off-target uptake pathway (but not limited to off-target uptake pathways necessarily) that is causing damage to nonneoplastic cells, by applying an effective amount of a copolymer with electrostatic and steric mediating properties applied to cells that are affected by said toxicity.

Without being bound to any particular theory, the utility of the invention can be manifest through interferences, mitigations, and inhibitions as described herein.

Another related approach managing ADC toxicity that occurs locally through off target uptake is to use an antibody to the ADC itself. The Antibody crafted toward the specific ADC will be designed to with a passivation moiety such as pegylation that would thereby decrease the ability of the ADC to enter cells via macropinocytosis. The ADC will, for example, be delivered locally to inhibit pinocytotic uptake. Antibody may be pegylated or have other passivation moieties as described herein using modifications to the antibody against the ADC by joining it to hydrophilic polymers for use in cell culture, labs, and in vivo, humans. Use in the laboratory is important because in embodiments where copolymers or antibodies are shown to reduce ADC related toxicity or other drug corneal toxicity, clinical development can be pursued with less risk for eye adverse events that complicate therapy.

The cationic graft copolymers interfere with cellular (corneal epithelial cell) uptake of the ADC. This unique methodological approach allows for improved corneal epithelial health and improved examination and symptomatic findings and decreases ocular risk. Reduced ADC-related cell death (e.g. toxicity) has many benefits. There are intravenous and other opportunities for toxicity reduction, thus claims are directed broadly. Megakaryocytes can use macropinocytosis to internalize ADCs and thus the opportunity to decrease this known adverse event associated ADCs is addressed with this invention.

Epithelial cells are known to have negative charges (3.6*10⁴) on the surface. Thus, the cationic graft and block copolymers passivate these negative charges which may play a role in ADC uptake. Evidence exists that making an ADC positively charged increases toxicity, and making it negatively charged decreases uptake. The key for this invention is the finding that passivation through electrostatic (or in some embodiments hydrophobic) interactions is beneficial. By decreasing the ADC's interaction at the cell surface, in one embodiment, ADC entry is reduced. By protecting the cell generally, ADC toxicity is reduced, as well. Other (non epithelial) cells have charged areas (and hydrophobic areas) allowing for these interaction reduction interferences, as well. The copolymer also may act at key protein components on the cell and ADC to limit uptake.

It also relevant that there are multiple methods for getting the cationic graft copolymer (or other effective block or graft copolymer) to the target tissue. PLL-g-PEG has been shown safe for mammalian IV infusion. Eye drops and topical exposures have been shown safe. The polymer can reach the target limbal cells via intravenous infusion. Subconjunctival delivery is also a potential valuable approach to ameliorating toxicity as this approach provides both a reservoir and an approach to the limbal stem cells and the daughter cells including basal epithelial cells. The conjunctiva is also somewhat permeable to macromolecules. The intercellular spaces in the conjunctival epithelium are wider than cornea and therefore more permeable to larger molecules. Thus, topically delivered, PLL-g-PEG can gain access to the stem cells (precursor cells to corneal superficial epithelial cells) and reduce toxicity by interfering with ADC corneal toxicity. Topical delivery to the cornea (or eye) can interfere with any ADC that gains entry to the superficial corneal or conjunctival epithelium. Thus, the release of the payload and its subsequent movement through the 5 or 6 corneal layers to basal cells is reduced. Also, larger molecules can naturally gain entry into the superficial epithelium. Thus, PLL-g-PEG or other identified and claimed graft or block copolymers can protect basal epithelial cells which are deeper in the epithelial layer. They have access to this space. Likewise, an ADC may gain exposure to deeper corneal epithelial cells. The copolymer thus can interfere at the ADC to block interaction with cell capture processes and also interfere on the corneal cell surface to interfere with ADC entry into cells. These effects lead to an effective approach to reducing ADC related corneal toxicity. In the case of the corneal cells, the basal, and other potentially adversely affected cells are protected and show, to some measurable degree less toxicity and patients demonstrate fewer signs and symptoms of ADC corneal toxicity. The opportunity is profound and applies to other approaches to minimizing off-target ADC toxicity. The corneal cells typically do not exhibit receptors for which the ADC is targeting, thus the toxicity is off target. In some embodiments, if there is a component of on target uptake based on the presence of a receptor on the corneal cell, these copolymers may be of benefit with a local application.

Delivery can be local, regional, or systemic. Many formulations exist. The copolymer (PLL-g-PEG) can decrease exposure of corneal tissue to the ADC an also reduce uptake. The interference is steric and charge based. By passivating the ADC and passivating the cell surface, uptake is reduced. The active charged or hydrophobic moiety adheres to the cell or ADC. The hydrophilic component of the polymer reduces ADC—corneal interactions and reduces uptake. Binding to conjunctiva or cornea by the copolymer can also serve as a reservoir for eventual interference and passivation of the ADC as it moves from the tear film into the lacrimal drainage system. The copolymers selected are nontoxic. Concentration in eye drops can range from 0.01 to 10% but are more commonly 0.5 to 3% w/w. Subconjunctival formulations may be of higher concentration.

The graft copolymer tested is PLL(20 kDa)-g[3.5]-PEG(5 kDa) in one experiment, but other molecular sizes and grafting ratios are safe and effective.

Formulations described herein also provide a safe, at some point, it may be beneficial to include other drug or polymer components in an eye drop for ADC-toxicity mitigation or viral protection. Subconjunctival delivery may require less frequent dosing. Topical delivery is compatible with long term use. A preservative is avoided in many embodiments to help protect the corneal surface and not harm epithelial cells otherwise. Exemplary additional active pharmaceutical ingredients for ophthalmological uses include, but are not limited to, lubricants and demulcents and sterile water and other standard excipients. There may be a benefit to combine with another active agent in some embodiments including novel protective agents for which a synergy with copolymers may exist. Also, antibiotics (fluoroquinolones, vancomycin, cephalosporin, gentamycin, crythromycin, azithromycin, sulfa drugs, bacitracin, gatifloxacin, levofloxin, moxifloxacin, ofoxacin, polymyxin B sulfate), acetazolamide, antazoline, aspirin, atropine, azelastine, bacitracin, betaxolol, bimatoprost, botanical drugs including zeaxanthine lutein, lycopene brimonodine, brinzolamide, carbachol, carteolol, ciprofloxacin, ofloxacin, cromalyn, cyclosporine, dapiprazole, dexamethasone, diclofenac, dipivifren, dorzolamide, epinephrine, erythromycin, fluoromethalone, flurbiprofen, gentamycin, glaucoma medications (prostaglandins, carbonic anhydrase inhibitors, epinephrine or alpha-agonists, beta-blockers), gramicidin, homatropine, hydrocortisone, hyoscine, keterolac, ibuprofen, ketotifen, latanaprost, levobunolol, levocabastine, levofloxin, loteprednol, medrysone, methazolamide, metipranolol, naphazoline, natamycin, nedocromil, neomycin, neuroprotective agents, nonsteroidal anti-inflammatoires, nepafanec, norfloxacin, ofloxacinm olopatadine, oxymetazoline, pemirolast, pheniramine, phenylephrine, pilocarpine, povidone, prednisolone, proparacaine, scopolamine, tetracaine, steroids, sulfacetamide, tetrahydrozoline, hypertonic tears, timolal, tobramycin, travaprost, trifluridine, trimethiprim, tropicamide, unoprostone and zinc-all may have some value in a co-formulation. Prodrugs and related compounds, as well as any new active pharmaceutical ingredients can be used with the block and graft copolymers here described to reduce ADC-toxicity or better manage ADC-related adverse events. Coformulations with the above molecules represent important drug products.

Multifunctional graft copolymers can also be used topically dermatologically to mitigate peripheral neuropathy that is more often seen with some types of payloads including for example MMAE. Dermatologic ointments, creams, sprays, lotions, silicone based drug products, aqueous based drug products, and lipid or oil based formulations including multifunctional graft copolymers including PLL-g-PEG can be utilized in this setting and are an embodiment of this invention for non-ophthalmic use. The sizes and MWs of the copolymers can be adjusted and wide polydispersity ranges can be used if efficacious. Excipients and other molecules and methods to enhance dermal penetration are included in any dermatologic formulations in some embodiments.

Based on the mechanism and non-specific protective and passivation approach, and ADC with a tubulin inhibitor or epithelial cell toxic agent is mitigated with this copolymer based approach. The following ADCs are claimed in this invention, but in no way is this list limiting. Copolymers discussed herein can be formulated with the ADC itself, or separately. The toxic payload may be a maytansinoid or an auristatin but may be another type generally, or another tubulin inhibitor. Tubulin inhibitors, and inhibitors of tubulin polymerization have specific utility in the current embodiments. Tubulin inhibitors may include but are not limited to: paclitaxel, epothilone, docetaxel, discodermolide, colchicine, combrestatin, 2-methoxyestradiol, methoxy benzenesulfonamides (E7010), vinblastine, vincristine, vinorelbine, vinfluine, dolastatins, halichondrins, hemiasterlins, cryptophysin 52, paclitaxel sites, vinca alkaloid sites, colchicine sites, etc. are considered. It may be a tubulin disruptor or work by other cytotoxic mechanisms. DNA synthesis inhibitors such as cytarabine are included. Other antineoplastic agents that can serve as payloads including Monomethyl auristatin E (MMAE), DM1 (mertansine), T-DM1, Maytansinoids, auristatins, DUO Duostatin-5 and other Duostatins, AF-HPA (auristatin F-hydroxypropylamide), PBD (Pyrrolobenzodiazepines), MMAF (Monomethyl auristatin F), Calich, caliche, calicheamicin, DM4 (Ravtansine), SN-38, Irinotecan metabolite, PF063801 01, Dxd, DNA topoisomerase I inhibitor, DOX, doxorubicin, PF063801 01, mitoxantrone, ctoposide, tesirine, PBD dimer, Pyrrolobenzodiazepine, SG3199. Toxins Targeting Tubulin Filaments, toxins Targeting DNA, toxins Targeting RNA, Nanocarriers, Protein Toxins, and enzymes are considered.

Varying linkers in ADC drugs are claimed, both known in 2020, and those that will be developed are embodiments when utilized with protection or treatment from toxicity through copolymer utilization in models or clinically. Embodiments also include but are not limited to use with ADCs noted below:

Gemtuzumab ozogamicin, Brentuximab vedotin, Trastuzumab emtansine, Inotuzumab ozogamicin, Polatuzumab vedotin-piiq, Enfortumab vedotin, Trastuzumab deruxtecan, IMGN242 (huC242-DM4), CanAg/DM4/SPDB, IMGN242 (huC242-DM4), CanAg/DM4/SPDB, Trastuzumab emtansine, (T-DM1), SAR3419 (huB4-DM4), SGN-CD19A, CD19/MMAF, (auristatin)/mc AVE9633, belantomab mafodotin, CD33/DM4/SPDB, CD70/MMAF (auristatin SGN-75 CD70-positive CD70/MMAF (auristatin)/mc, SAR566658 CA6+, DS6/DM4/SPDB, CD33/calicheamicin/Hydrazine, Ephrin type A receptor 2 (EphA2)/mcMMAF (auristatin)/mc, Lorvotuzumab, mertansine, D56/DM1/SPP, CD138/DM4/SPDB, FRα/DM4/SPDB, AGS-16M8FMMAF, AGS-16C3FMMAF, ENPP3/MMAF (auristatin), as well as, without being limiting:
A166 ADC
AB-3A4 ADC
ABBV-176 (ABV176)
ABBV-321
ABBV-3373
AbGn-107 (Ab1-18 Hr1)
AbGn-108
AbGn-110
ADCT-502
ADCT-601
ADCT-602 (hLL2-cys-PBD)
ADCT-701
AGS-16C3F (AGS 16C3F/AGS-16M8F)
AGS-16M8F (AGS 16C3F/AGS-16C3F)
AGS62P1
ALT-P7
AMG 224
AMG-595
AMG172
Anetumab corixetan
Anetumab Ravtansine| BAY 94-9343
Anti-ADAM9 ADC (Anti-ADAM9-sulfoSPDB-DM4)
Anti-ADAM9 ADC (Anti-ADAM9(C442)-DGN549)
Anti-CD19 ATAC (anti-CD19 ADC)
Anti-CD22-NMS249
Anti-CD70-ADC (CD70-ADC)
Anti-cMET ADC (CBT-161)
Anti-endosialin-MC—VC-PABC-MMAE
Anti-ETBR (RG-7636)
anti-HER-3 ADC
anti-TEM-1 Antibody-Drug Conjugate (TEM-1-ADC).
Anti-TM4SF1 ADC
Aprutumab ixadotin| BAY 1187982
AR-001| YBL-001
ARX517-PSMA-ADC
ARX788 HER2 ADC| ARX788
ASG-22CE
ASN-004 (ASN 004)
AVID100
AVID300| A VID 300
Azintuxizumab Vedotin| ABBV-838
Azonafide-ADC
BA3011| CAB-Axl-ADC (CAB-anti-Axl-ADC)
BA3021| Anti-ROR2 ADC| CAB-ROR2-ADC
BAT8001
BAY79-4620
BDC-1001
Belantamab mafodotin| GSK2857916| J6MO-mcMMAF
BIIB-015| BIIB015
Bivatuzumab mertansine| Anti-CD44v6-DM1| BIWI-1
BL-B029A1
BL-M002A2
BL-M005A2
Brentuximab vedotin| SGN35| Adcetris®
Bstrongximab-ADC
BT1718 (BT 1718)
c-MET ADC
Camidanlumab tesirine| ADCT-301| HuMax-TAC-ADC
Cantuzumab mertansine| huC242-DM1| SB-408075
Cantuzumab Ravtansine| IMGN-242
CC-99712-Anti-BCMA ADC
CD184-Dasatinib (CD184-Dasatinib-ADC)
CD184-FK506
CD22-4AP
CD70-glucocorticoid ADC
CDX-014
Cetuximab sarotalocan| ASP-1929| RM-1929
Cirmtuzumab Vedotin| UC-961ADC3
Clivatuzumab Tetraxetan
CMB-401| hCTMO1-calicheamicin| CDP-671
Cofetuzumab pelidotin| PF-06647020
Coltuximab Ravtansine| SAR 3419
CX-2009
Cymac-001 (anti-CD163-dexamethasone ADC)
D3-GPC2-PBD (anti-GPC2 ADC)
DEDN6526A (RG-7636/RG7636)
Denintuzumab mafodotin| SGN-CD19A| SGN-19A| hBU12-491
Depatuxizumab Mafodotin| ABT-414
Derlotuximab Biotin
DHES0815A
Disitamab Vedotin| RC-48| RC 480-ADC| RC48
DMOT4039A (DMOT-4039A/RG7600/RG 7600)
DS-1062 (TROP2 ADC)
DS-7300 (B7-H3 ADC)
DSTA4637S (Anti-S. aureus TAC; RG7861)
EC1169
EC2629
EDC1 (DYS-ADC)
EDC9 (EDC-CD20)
EDO-772P/B776
EDO-B278
Enapotamab vedotin| HuMax-AXL-ADC| AXL-107-MMAE
Enfortumab Vedotin| ASG-22ME| ASG-22MSE
EP-400
Epitumomab cituxetan
Epratuzumab-SN-38
EV20/MMAF
Gemtuzumab ozogamicin| Mylotarg®
Glembatumumab vedotin| CDX-011
GM103
GTB-1550| OXS-1550| DT2219ARL
HDP-101 (Anti-CD269-ADC; Anti-CD269-amanitin-ADC)
Hertuzumab Vedotin
HKT288 (CDH6-ADC)
HTI-1511
HuMAB-5B1-ATAC
Ibritumomab tiuxetan
IGN523
IGN786
IKS01
IKS02
IKS03
IKS04
Iladatuzumab vedotin| DCDS0780A| RO7032005
IMAB027-vcMMAE
IMAB362-vcMMAE
IMB-201
IMB-202
IMGN 289
IMGN 779| IMGN779
IMGN-242
IMGN-388
IMGN-633 (AVE9633)
IMGN632| IMGN 632
IMMU-140 (anti-HLA-DR-SN-38 ADC)
Indatuximab Ravtansine| BT-062
Indusatumab Vedotin| MLN-0264| TAK-264
Inotuzumab ozogamicin (CMC-544)| BESPONSA®
IPH43
KTN0125
KTN0182A
Labetuzumab govitecan| IMMU-130
Ladiratuzumab vedotin| SGN-LIVIA| Anti-LIV-1 ADC
Laprituximab emtansine| IMGN-289| IMGN289
LCB14-0110 (Herceptin-LC-LBG-MMAF)
LCB14-15 nm
LCB14-15xx
LCB14-15xx (NNV019)
LCB14-17nn
LCB14-19 nm
LCB14-2 nm
Lifastuzumab Vedotin| RG-7599| DNIB0600A
Lilotomab satetraxetan
Loncastuximab tesirine| ADCT-402
LOP628 (LOP-628)
Lorvotuzumab mertansine| IMGN-901
Losatuxizumab vedotin| ABBV-221
Lupartumab amadotin| BAY 1129980
LY3076226
MDX-060/iratumumab
MDX-1203| BMS936561
MEDI-547
MEDI3726| ADCT-401
MEDI4276 (MEDI 4276)
MEN1309| OBT076
MGC018; Anti-B7-H3 ADC
MI130004
Milatuzumab doxorubicin (hLL1-DOX| IMMU-110)
Mirvetuximab Soravtansine| IMGN-853
Mirzotamab Clezutoclax| ABBV-155
MLN-2704| MLN2704
MM-302
MORAb-202
Naratuximab emtansine| IMGN529| K7153A| Debio 1562
NBE-001
NBE-002-ROR-1
NBE-003
NC-6201 (ADCM-E7974)
NV101 (Doxorubicin-anti-CD99)
NV102 (Doxorubicin-anti-CD19)
NV103 (Irinotecan-anti-CD99)
OBI-999| Anti-Globo H ADC
OMTX503 (Anti-MTX3:Nigrin Immunoconjugate)
OMTX705 (Anti-MTX5:Cytolysin ADC)
Patritumab Deruxtecan
PCA062 (PCA-062)
PEN-221
PF 06263507 (A1-mcMMAF| Anti-5T4 monoclonal antibody| PF-06263507| PF06263507)
PF-06647263 (anti-EFNA4-ADC)
PF-06650808 (Anti-NOTCH3 ADC)
PF-06664178| PF06664178| PF 06664178
PF-06688992| PF06688992
PF-06804103 (Anti-NG-HER2 ADC)
Pinatuzumab vedotin| RG-7593| DCDT2980S| DCDT-2989S
Polatuzumab vedotin| Polivy™ | RG-7596| DCDS4501A| DCDS-4501A
Praluzatamab Ravtansine
PSMA-ADC
Q5-Drug Conjugate
REGN2878-DM1 (Anti-PRLR-ADC)
RG-7598 (DFRF 4539A/RG7598/RG 7598)
RG-7841 (Anti-Ly6E/DLYE5953A)
RG7986
Rolinsatamab talirine
Rovalpituzumab tesirine| Rova-T| SC0001
Sacituzumab govitecan| IMMU-132| hRS7-SN38
Samrotamab Vedotin| ABBV-085| PR-1498487-MMAE
SAR 566658 (SAR566658)
SAR408701| SAR 408701
SAR428926
Satoreotide tetraxetan
Satumomab Penditide (OncoScint® CR/OV)
SC-006
SC16LD6.5
Serclutamab Talirine
SGN CD70 A| SGN-CD70A (superseding SGN-75)
SGN-15| BMS-182248| BR96-DOX
SGN-CD123A
SGN-CD19B
SGN-CD352a
Sirtratumab vedotin (ASG-15ME)
SNG-8023 ADC
Sofituzumab vedotin| Anti-MUC16 ADC| RG7458| DMUC5754A
ST1
STRO-001 (Anti-CD74-ADC)
STRO-002
Tabituximab Barzuxetan| OTSA-101-DTPA
Tacatuzumab Tetraxetan
Tamrintamab Pamozirine| Anti-DPEP3 ADC
Telisotuzumab Vedotin| ABBV 399| ABBV399
TGM-001
TGM-002
TGM-003
TGM-004
TGM-005
Tisotumab Vedotin| HuMax®-TF-ADC
Trastuzumab deruxtecan (DS-8201, DS-8201a)
Trastuzumab duocarmazine| SYD985
Trastuzumab Emtansine| T-DM1| Kadcyla®
TRPH-222 (CD22-4AP)
U3-1402| HER3 ADC
Upifitimab Rilsodotin (UpRi)
Vadastuximab talirine| SGN-CD33A
Vandortuzumab vedotin (RG7450; DSTP3086S)
VIS705
Vorsetuzumab mafodotin (SGN-75)
VYNFINIT® | Vintafolide (EC145/MK 8109)
XMT-1522| TAK-522
XMT-1536
ZV05-ADC (5T4-MMAF ADC)
ZV203

New and as yet undescribed publicly ADCs with the potential for corneal toxicity may be treated with the methods of this invention.

Minimization of cytarabine corneal toxicity is also claimed as there is uptake of the molecule with a positive charge on the nitrogen, and PLL-g-PEG and other cationic graft copolymers can minimize this charged interaction by passivating negative charges on surface cells.

Based on physical chemistry the copolymers will be effective in embodiments herein because said copolymer will have access to the drug (pharmaceutical), solution bathing the cells at risk, and the cells themselves creating a dynamic protective environment. When applied as a drop, suspension, solution, controlled delivery system or powder (possibly lyophilized), the graft co-polymer is able to move from surface tissue to ADC or other pharmaceuticals in solution or tears or extracellular fluid due to physical chemistry on-off bonding associated with electrostatic or hydrophobic interactions.

From experiments described herein, it is expected that formulations of the present invention will also be useful for preventing viral infection or ADC uptake or interaction with target cells including, but not limited to, skin, mucous membranes (eye, nasal, oral) and hair. These formulations can thus also be applied in accordance with the present invention to epithelial tissue of the eye, respiratory tract, and gastrointestinal tract, mucous membrane, exposed wound surfaces, corneal and conjunctival surfaces, skin, and surgical and traumatic wounds and ulcerations. These formulations can serve to protect skin and other organs from viral infection and unwanted ADC interaction. Benefits can include reduction in rate of infection, less severe infections, and reduction in corneal epithelial toxicity. The copolymer may interact with both the infective agent or pharmaceutical and the surface of the cell at risk to passivate any interaction and provide a protective effect.

For these uses in humans, the formulation may be in the form of a lotion, gel, liquid, spray, rinse, dissolvable wafer, or glycerin bar to which water is added to solubilize the graft co-polymer or block co-polymer to make it more amenable to application. Formulations can be provided as individual, or single use products or in volumes for industrial use and/or multiple use dispensers. In addition to the graft or block co-polymer, such formulations may comprise any and all typical binders, excipients, and components found in cosmetic sprays, lotions, soaps, shampoos, cleansers, and oral, nasal, and eye care products.

Formulations can also be used in accordance with the present invention on animals, including household pets, to decrease viral infection or ADC toxicity.

Other uses for these formulations will become evident to those skilled in the art upon reading this disclosure and are such uses are encompassed by the present invention.

Extended-release formulations may prove particularly beneficial and herein are encompassed.

PLL-g-PEG is an example of a copolymer with bioadhesive and passivation moieties. PLL-g-PEG uses electrostatic bioadhesion with the cationic backbone. Hydrophobic and anionic moieties utilize alternative bioadhesive interactions (hydrophobicity, anionic). Formulations of said copolymers are safe for human use.

Furthermore, tisotumab-vedotin-tftv, has toxicity that may be driven in part by an on target mechanism (tissue-factor which is expressed on certain epithelial cells in certain conditions), and topically applied multifunctional graft copolymers mitigate and reduce the corneal related adverse events associated with this therapeutic by interfering with any on target binding as well as macropinocytotic uptake which will still be present, as that process is natural to corneal epithelial cells. Other polymers contemplated herein, including poloxamers and cationic based large polymeric structures, have a beneficial effect as well and opportunities for their use in these protective settings are identified and may form the basis of future claims. Trastuzumab based ADCs are another example where on target toxicity may play a role and can be mitigated for epithelial cells at risk of toxicity. A multifunctional graft copolymer can interfere with IgG1 binding by ABT-414.

The following nonlimiting examples are provided to further illustrate the present invention. Percentages are weight/weight percent.

It has been described that drugs may be secreted in tear fluid. From: Lec, Brian A., et al. “Clinical and Histological Characterization of Toxic Keratopathy From Depatuxizumab Mafodotin (ABT-414), an Antibody-Drug Conjugate” Cornea (2018). Regarding Microcyst-like epithelial keratopathy (MEK): “Steroid is not an adequate treatment.” And: “ . . . confocal microscopy was notable for multiple large, round, hyperreflective lesions throughout the epithelium that seemed to correlate with MEK seen clinically. Histologically, the microcysts seem to correlate with engulfed apoptotic cells throughout the epithelium. In addition to the increased apoptotic cells noted in the histological specimens, immunohistochemistry revealed IgG-positive intracytoplasmic granules in the basal epithelium. Because depatuxizumab, the antibody component of ABT-414, is a monoclonal IgG1, it suggests that ABT-414 itself is deposited within the basal epithelium. The presence of ABT-414 directly in the epithelium in turn likely explains the increased apoptosis seen on histology.”

Experimental techniques adapted from: Zhao, Hui, et al. “Modulation of Macropinocytosis-Mediated Internalization Decreases Ocular Toxicity of Antibody-Drug Conjugates.” Cancer research 78.8 (2018): 2115-2126. Study design for cell culture: “Cell lines and reagents. All cells were maintained according to vendors' protocol.

Human primary corneal epithelial cells (HCEC) from Life Technologies (catalog no. C018-5C) were cultured in keratinocyte-SFM (catalog no. 17005-42), and HCEC cells from ATCC (catalog no. PCS-700-010) were cultured in corneal epithelial cell basal medium (catalog no. PCS-700-030) supplemented with corneal epithelial cell growth kit (catalog no. PCS-700-040). Human umbilical vein endothelial cells (HUVEC, catalog no. C-003-5C) and human dermal fibroblasts, adult (HDFa, catalog no. C-013-5C) were from Life Technologies. HUVECs were grown in Medium 200 supplemented with low serum growth supplement (LSGS, catalog no. S-003-10). HDFa cells were grown in Medium 106 supplemented with LSGS. KU812 cells were from ATCC (catalog no. CRL-2099) and were grown in RPMI1640 plus 10% FBS as described previously. Cell lines were passaged in our laboratory for fewer than 6 months after their resuscitation. Human cell lines were confirmed utilizing short tandem repeat profiling (Promega) and confirmed to be Mycoplasma negative. Reagents for human hematopoietic stem cells (HSC) and their differentiation to megakaryocytes were reported previously. T-DM1 (Kadcyla; Genentech/Roche) was purchased (Myoderm).

Macropinocytosis HSCs (10⁵ cells/well) were grown in 24-well plates overnight and then incubated with 1 mg/mL dextran-FITC (10,000 MW, Life Technologies) for 3 hours at 37° C. or 4° C. as control. Cells were detached with trypsin and neutralized with neutralization solution (Life Technologies, catalog no. R002100). Cells were then washed three times with FACS stain buffer (FBS, BD Pharmingen, catalog no. 554656) and analyzed by Attune acoustic focusing cytometer (Life Technologies). To test the effect of 5-(N-ethyl-Nisopropyl) amiloride (EIPA), the indicated amount of EIPA was added to the cell culture 30 minutes prior to dextran-FITC addition. Median fluorescence intensity ratio (MFIR) was derived from MFI values at 37° C. normalized against those at 4º° C.

### Proliferation Assays

HCECs (500 cells/well) and HUVECs (2,000 cells/well) in 100 mL were seeded in collagen-coated 96-well plates (Corning, catalog no. 354650), and HDFa (2,000 cells/well) and KU812 (2,500 cells/well) in 100 mL were grown in 96-well tissue culture plates (Corning assay plate, catalog no. 3903). After treatment with ADCs for 6 days, CellTiter-Glo (CTG) luminescence assay kit (Promega, catalog no. G7572) was used to measure the viability of the treated cells relative to control. CTG values were normalized against mock-treated cells at day 6 (% max proliferation), and GraphPad Prism 6 was used to generate IC50 values using a sigmoidal dose-response (variable slope). Assay plates contained technical triplicates for each drug concentration, and data presented are the mean of at least two independent determinations.

### ANS Assay

ADCs (2 mg/mL) were prepared in PBS, and 1:2 serial dilutions were made in a black-walled, 96-well plate. Equal volume of 1,8-ANS (1-anilinonaphthalene-8-sulfonic acid, Thermo Fisher Scientific, catalog no. A47) was added and incubated for 30 minutes at room temperature. Fluorescence signal was measured (ex. 390 nm/em. 470 nm). Hydrophobicity index was the slope from the linear regression analysis.

### Confocal Microscopy

Cells were seeded on 8-well chamber slides (0.75 105 cells per well) and cultured for 48 hours prior to treatment and subsequent immunostaining. Cells were then incubated with AGS-16C3F with and without coincubation of 0.5 mg/mL Dextran-Texas Red (Molecular Probes; D18653) for 4 hours at 37° C.

Inhibition of macropinocytosis was evaluated by treating cells with EIPA for 30 minutes prior to AGS-16C3F/Dextran-Texas Red incubation. After the incubation period, unbound antibody was washed off with PBS and cells were fixed in 4% paraformaldehyde for 20 minutes at room temperature. Cells were then permeabilized in PBS plus 0.1% Triton-X-100 for 15 minutes, and nonspecific labeling was blocked in PBS plus 10% normal goat serum. Cell surface-bound and internalized cytosolic AGS-16C3F was visualized by incubating cells with Alexa Fluor 488-labeled goat anti-human IgG (Thermo Fisher Scientific, catalog no. A-11013). Nuclei were visualized with TO-PRO-3 Iodide (Thermo Fisher Scientific, catalog no. T3605), and coverslips were mounted using ProLong Gold Antifade reagent (Thermo Fisher Scientific, catalog no. P36934) for imaging. High-resolution laser confocal image sections were acquired using a Leica TCS SP5 II (63× oil immersion objective; NA ¼ 1.4) and were scanned sequentially to minimize fluorophore cross-talk and false-positive colocalization.

And the rabbit techniques were also adapted from Zhou et al: “Animal studies and welfare In vivo xenografted tumor models and pharmacodynamic studies were carried out as described before. . . . Male Dutch Belted [Haz:(DB)SPF] rabbits weighing approximately 1.5 to 2.0 kg were used for ocular tolerability studies. Rabbits were acclimated for at least 6 days prior to first dose. All animals were housed in individual, suspended, stainless steel caging, were provided feed and water, and were maintained under environmental conditions in compliance with all animal welfare guidelines. Test articles were administered to groups of 3 to 4 rabbits intravenously via a marginal ear vein, followed by a saline flush once weekly for up to six doses (days 1, 8, 15, and 22). General health was assessed by weekly measurement of body weight and cage-side observations. Ocular tolerability was assessed by external examination-slit lamp biomicroscopy to examine the adnexa and anterior portion of each eye. In addition, the ocular fundus was examined using an indirect ophthalmoscope following dilation with a mydriatic agent. Corneal fluorescein staining was also performed to examine corneal damage. A fluorescein solution (approximately 1 mg/mL) was applied to the cornea by a cotton-tipped swab. At necropsy, eyes and other selected tissues were placed in fixative according to established procedures for IHC.”

All references, publications, and patents are incorporated herein in their entirety.

### Specific Embodiments

A list of additional and related embodiments of the present invention follow.

In one embodiment, the present invention provides a method for decreasing viral infectivity by treating tissues that are involved with transfection with an effective amount of a graft or block copolymer with either cationic, hydrophobic, or anionic moieties and a hydrophilic passivation moiety. In certain embodiments, the copolymer is PLL-g-PEG. In other embodiments, the graft co-polymer of said formulation comprises a cationic backbone and side chains that are water soluble and non-ionic. In some embodiments, the block co-polymer of said formulation comprises at least one cationic block and at least one water soluble and non-ionic block. In other embodiments, the block co-polymer of said formulation comprises at least one block which is hydrophobic and at least one block which is water soluble and anionic, cationic or non-ionic.

In one embodiment, the present invention provides a method for decreasing viral infectivity by treating tissues that are involved with transfection with an effective amount of a graft or block copolymer with either cationic, hydrophobic, or anionic moieties and a hydrophilic passivation moiety; wherein the biological surface to which the formulation of copolymer is administered is a mucous membrane selected from ocular mucosa, oral mucosa, nasal mucosa and respiratory tract mucosa, respiratory tract epithelium, genitourinary mucosa, gastrointestinal mucosa of a subject. In certain embodiments, the biological surface to which the formulation of copolymer is administered is a surface of an eye.

In one embodiment, the present invention provides a method for decreasing viral infectivity by treating tissues that are involved with transfection with an effective amount of a graft or block copolymer with either cationic, hydrophobic, or anionic moieties and a hydrophilic passivation moiety; wherein the viral infection is selected from coronaviruses, influenzas viruses, Ebola viruses, and novel viruses transmitted through mucous membrane exposure. In other embodiments, the virus is SARS-COV-2.

In one embodiment, the present invention provides a method for decreasing viral infectivity by treating tissues that are involved with transfection with an effective amount of a graft or block copolymer with either cationic, hydrophobic, or anionic moieties and a hydrophilic passivation moiety; wherein the graft co-polymer or block co-polymer of said formulation comprises 0.001 to 40% of said formulation.

In one embodiment, the present invention provides a method for decreasing viral infectivity by treating tissues that are involved with transfection with an effective amount of a graft or block copolymer with either cationic, hydrophobic, or anionic moieties and a hydrophilic passivation moiety; wherein the graft co-polymer or block co-polymer of said formulation comprises 0.1 to 10% of said formulation.

In one embodiment, the present invention provides a method for decreasing viral infectivity by treating tissues that are involved with transfection with an effective amount of a graft or block copolymer with either cationic, hydrophobic, or anionic moieties and a hydrophilic passivation moiety; wherein the viral infection is selected from coronaviruses, influenzas viruses, Ebola viruses, and novel viruses transmitted through mucous membrane exposure and the passivation effect is based on interference with the SARS-Cov-2 spike protein and ACE2 receptor on at risk cells.

In one embodiment, the present invention provides a method for decreasing viral infectivity by treating tissues that are involved with transfection with an effective amount of a graft or block copolymer with either cationic, hydrophobic, or anionic moieties and a hydrophilic passivation moiety; wherein the viral infection is selected from coronaviruses, influenzas viruses, Ebola viruses, and novel viruses transmitted through mucous membrane exposure and where the therapeutic effect is a general steric inhibition. The use of mucopenetration enhancers or mucopenetration technology combined with a multifunctional graft or block copolymer to decrease vial infectivity, either sequentially or combined in a single formulation or coformulation is an embodiment of this invention. Coformulation of a set of at least one graft or block copolymer combined with another active agent that can help reduce cell transfection from viral sources is an embodiment of this invention.

In one embodiment, the present invention provides a method to decease the adverse events associated with antibody-drug conjugate usage that is causing damage to (adversely affecting) nonneoplastic cells, by applying an effective amount of a copolymer with electrostatic and steric mediating properties to such cells and tissues (those that are adversely affected by antibody-drug conjugate usage). In another embodiment, the copolymer is selected from cationic graft, cationic block, hydrophobic graft, hydrophobic block, anionic graft, and anionic block copolymers. In yet another embodiment, the copolymer is formulated in one or more of the following approaches: powders, solutions, suspensions, topical preparations, intravenous preparations, oral preparations, oral rinses, nasal sprays, eye drops. In further embodiments, the percentage of the copolymer solution is at minimum 0.01% by weight. In other embodiments, the percentage of the copolymer solution is at maximum 40% by weight for solutions and suspensions.

In one embodiment, the present invention provides a method to decease the adverse events associated with antibody-drug conjugate usage that is causing damage to (adversely affecting) nonneoplastic cells, by applying an effective amount of a copolymer with electrostatic and steric mediating properties to such cells and tissues (those that are adversely affected by antibody-drug conjugate usage) wherein the copolymer is PLL-g-PEG. In further embodiments, the copolymer is selected from the list of combinations described in the application supra.

In one embodiment, the present invention provides a method to decrease ADC related corneal epithelial toxicity by administering a copolymer with bioadhesive and passivation components to cells at risk of off target drug uptake. In other embodiments, the copolymer is applied to corneal and conjunctival epithelial cells. In further embodiments, the copolymer is PLL-g-PEG. In certain embodiments, the formulation is a solution for delivery to the subconjunctival space.

In one embodiment, the present invention provides a method to decease the adverse events associated with antibody-drug conjugate usage in a human whereby an effective amount of a copolymer with electrostatic and steric mediating properties is applied to cells that are involved in said adverse events. In other embodiments, the copolymer is selected from cationic graft, cationic block, hydrophobic graft, hydrophobic block, anionic graft, and anionic block copolymers. In yet other embodiments, the copolymer is selected from polymers in this disclosure supra.

In one embodiment, the present invention provides a method to decrease microcyst-like epithelial toxicity associated with cytotoxins cleaved from ADCs by applying to corneal epithelial cells an effective amount of a copolymer with bioadherence and passivation properties. In other embodiments, the copolymer is PLL-g-PEG.

In one embodiment, the present invention provides a method for decreasing rates and severity of ocular adverse events associated with ADC use by delivering to the eye a copolymer with bioadhesive and passivation moieties prior to initiation of systemic ADC therapy. In other embodiments, the present invention provides a method for decreasing rates and severity of ocular adverse events associated with ADC use by delivering to the eye a copolymer with bioadhesive and passivation moieties prior to initiation of systemic ADC therapy wherein the copolymer is PLL-g-PEG.

In one embodiment, the present invention provides a method for improving signs and symptoms of ocular adverse events associated with ADC use by delivering to the eye a copolymer with bioadhesive and passivation moieties after initiation of systemic ADC therapy. In other embodiments the present invention provides a method for improving signs and symptoms of ocular adverse events associated with ADC use by delivering to the eye a copolymer with bioadhesive and passivation moieties after initiation of systemic ADC therapy wherein the copolymer is PLL-g-PEG.

In one embodiment, the present invention provides a method to reduce ADC uptake into one of limbal stem cells, transient amplifying cells, basal epithelial cells, wing cells, and corneal epithelial cells, by exposing said cells to a copolymer with bioadhesive and passivation moieties prior to exposure to the ADC. In other embodiments, the present invention provides a method to reduce ADC uptake into one of limbal stem cells, transient amplifying cells, basal epithelial cells, wing cells, and corneal epithelial cells, by exposing said cells to a copolymer with bioadhesive and passivation moieties prior to exposure to the ADC wherein the copolymer is PLL-g-PEG.

In one embodiment, the present invention provides a method to reduce ADC uptake into one of limbal stem cells, transient amplifying cells, basal epithelial cells, wing cells, and corneal epithelial cells by exposing said cells to a copolymer with bioadhesive and passivation moieties after exposure to the ADC. In other embodiments, the present invention provides a method to reduce ADC uptake into one of limbal stem cells, transient amplifying cells, basal epithelial cells, wing cells, and corneal epithelial cells by exposing said cells to a copolymer with bioadhesive and passivation moieties after exposure to the ADC wherein the copolymer is PLL-g-PEG.

In one embodiment, the present invention provides a method to reduce ADC uptake by macropinocytosis into one of limbal stem cells, limbal epithelial cells, limbal epithelial daughter cells, transient amplifying cells, basal epithelial cells, wing cells, corneal epithelial cells, and differentiated corneal epithelial cells by exposing said cells to a copolymer with bioadhesive and passivation moieties prior to or subsequent to exposure to the ADC, using a formulation with an effective percentage based on weigh/weight calculations of said copolymer. In other embodiments, the present invention provides a method to reduce ADC uptake by macropinocytosis into one of limbal stem cells, limbal epithelial cells, limbal epithelial daughter cells, transient amplifying cells, basal epithelial cells, wing cells, corneal epithelial cells, and differentiated corneal epithelial cells by exposing said cells to a copolymer with bioadhesive and passivation moieties prior to or subsequent to exposure to the ADC, using a formulation with an effective percentage based on weigh/weight calculations of said copolymer wherein the copolymer is PLL-g-PEG.

In one embodiment, the present invention provides a method to reduce ocular adverse events associated with ADCs by treating a patient with a copolymer with bioadhesive and passivation moieties using an amount to be efficacious. In other embodiments, the present invention provides a method to reduce ocular adverse events associated with ADCs by treating a patient with a copolymer with bioadhesive and passivation moieties using an amount to be efficacious, wherein the copolymer is PLL-g-PEG.

In one embodiment, the present invention provides a method of using copolymers demonstrating electrostatic and stearic interactions at the cellular level to minimize adverse effects caused by exposure of those cells to factors selected from: SARS-Cov-2, novel viruses, viruses in the setting of an epidemic, ADCs with cytotoxic payloads which can lead to human pathology and morbidity. In other embodiments, the present invention provides a method of using copolymers demonstrating electrostatic and stearic interactions at the cellular level to minimize adverse effects caused by exposure of those cells to factors selected from: SARS-Cov-2, novel viruses, viruses in the setting of an epidemic, ADCs with cytotoxic payloads which can lead to human pathology and morbidity wherein the copolymer is PLL-g-PEG.

In one embodiment, the present invention provides a method to reduce ocular toxicity due to systemic exposure of a human to ADCs with tubulin disruptors as the payload by treating the eye with an effective amount of cationic graft copolymer formulation. In other embodiments, the present invention provides a method to reduce ocular toxicity due to systemic exposure of a human to ADCs with tubulin disruptors as the payload by treating the eye with an effective amount of cationic graft copolymer formulation wherein the cationic graft copolymer is PLL-g-PEG. In yet other embodiments, the present invention provides a method to reduce ocular toxicity due to systemic exposure of a human to ADCs with tubulin disruptors as the payload by treating the eye with an effective amount of cationic graft copolymer formulation wherein the cationic graft copolymer is PLL-g-PEG and the treatment is through an eye drop formulation. In certain embodiments, the present invention provides a method to reduce ocular toxicity due to systemic exposure of a human to ADCs with tubulin disruptors as the payload by treating the eye with an effective amount of cationic graft copolymer formulation wherein the cationic graft copolymer is PLL-g-PEG, the treatment is through an eye drop formulation, and the cationic graft copolymer is PLL-g-PEG in a concentration in an eye drop formulation in a weight/weight range from 0.01% to 5%.

In one embodiment, the present invention provides a method to reduce dose holds and dose reductions of ADCs in the treatment of human malignancy by reducing corneal adverse events and ocular safety concerns through a PLL-g-PEG eye drop formulation applied to the eye in an effective amount in an at-risk patient.

In one embodiment, the present invention provides a method to decrease corneal cell toxicity caused by a pharmaceutical selected from the group of ADCs, biologics, small molecules, large molecules, peptides whereby a copolymer with bioadhesive and passivation components is applied locally to the eye at risk of adverse effects from said pharmaceutical. In other embodiments, the present invention provides a method to decrease corneal cell toxicity caused by a pharmaceutical selected from the group of ADCs, biologics, small molecules, large molecules, peptides whereby a copolymer with bioadhesive and passivation components is applied locally to the eye at risk of adverse effects from said pharmaceutical, wherein the copolymer is PLL-g-PEG.

In one embodiment, the present invention provides a method to decease adverse events associated with a preserved topical ophthalmic drug product by administering to a subject a formulation comprising said preserved topical ophthalmic drug product and further comprising a copolymer, wherein said copolymer displays electrostatic and steric mediating properties and is in an amount sufficient to decrease said adverse events associated with said administering to said subject said formulation comprising said preserved topical ophthalmic drug product in the absence of said copolymer. In other embodiments, the present invention provides a method to decease adverse events associated with a preserved topical ophthalmic drug product by administering to a subject a formulation comprising said preserved topical ophthalmic drug product and further comprising a copolymer, wherein said copolymer displays electrostatic and steric mediating properties and is in an amount sufficient to decrease said adverse events associated with said administering to said subject said formulation comprising said preserved topical ophthalmic drug product in the absence of said copolymer, wherein said copolymer is PLL-g-PEG. In yet other embodiments, the present invention provides a method to decease adverse events associated with a preserved topical ophthalmic drug product by administering to a subject a formulation comprising said preserved topical ophthalmic drug product and further comprising a copolymer, wherein said copolymer displays electrostatic and steric mediating properties and is in an amount sufficient to decrease said adverse events associated with said administering to said subject said formulation comprising said preserved topical ophthalmic drug product in the absence of said copolymer, wherein the copolymer is selected from cationic graft, cationic block, hydrophobic graft, hydrophobic block, anionic graft, and anionic block copolymers.

In certain embodiments, the present invention provides a method to decease adverse events associated with a preserved topical ophthalmic drug product by administering to a subject a formulation comprising said preserved topical ophthalmic drug product and further comprising a copolymer, wherein said copolymer displays electrostatic and steric mediating properties and is in an amount sufficient to decrease said adverse events associated with said administering to said subject said formulation comprising said preserved topical ophthalmic drug product in the absence of said copolymer, wherein said copolymer is PLL-g-PEG and the percentage of the copolymer solution is at minimum 0.01% by weight. In other embodiments, the present invention provides a method to decease adverse events associated with a preserved topical ophthalmic drug product by administering to a subject a formulation comprising said preserved topical ophthalmic drug product and further comprising a copolymer, wherein said copolymer displays electrostatic and steric mediating properties and is in an amount sufficient to decrease said adverse events associated with said administering to said subject said formulation comprising said preserved topical ophthalmic drug product in the absence of said copolymer, wherein said copolymer is PLL-g-PEG and the percentage of the copolymer solution is at maximum 40% by weight for solutions and suspensions.

In one embodiment, the present invention provides a method to decease adverse events associated with a preserved topical ophthalmic drug product by administering to a subject a formulation comprising said preserved topical ophthalmic drug product and further comprising a copolymer, wherein said copolymer displays electrostatic and steric mediating properties and is in an amount sufficient to decrease said adverse events associated with said administering to said subject said formulation comprising said preserved topical ophthalmic drug product in the absence of said copolymer, wherein said copolymer is the copolymer is selected from the list of combinations described in the application supra.

In one embodiment, the present invention provides a method to decease the adverse events associated with administration to a subject an antibiotic ophthalmic drug product selected from the group consisting of an antibacterial, an antifungal, an antiviral, an anti-helminthic, an anti-parasitic, an anti-acanthomoeba agent, and an antiseptic, the method comprising administering said antimicrobial ophthalmic drug products in a formulation further comprising a copolymer with electrostatic and steric mediating properties in a dose sufficient to decrease said adverse events in said subject relative to administration of said formulation lacking said copolymer with electrostatic and steric mediating properties.

In one embodiment, the present invention provides a method to decease the adverse events associated with administration to a subject of an antibiotic ophthalmic drug product selected from the group consisting of an antibacterial, an antifungal, an antiviral, an anti-helminthic, an anti-parasitic, an anti-acanthomoeba agent, and an antiseptic, the method comprising administering said antimicrobial ophthalmic drug products in a formulation further comprising a copolymer with electrostatic and steric mediating properties in a dose sufficient to decrease said adverse events in said subject relative to administration of said formulation lacking said copolymer with electrostatic and steric mediating properties wherein the said copolymer is selected from cationic graft, cationic block, hydrophobic graft, hydrophobic block, anionic graft, and anionic block copolymers.

In one embodiment, the present invention provides a method to decease the adverse events associated with administration to a subject of an antibiotic ophthalmic drug product selected from the group consisting of an antibacterial, an antifungal, an antiviral, an anti-helminthic, an anti-parasitic, an anti-acanthomoeba agent, and an antiseptic, the method comprising administering said antimicrobial ophthalmic drug products in a formulation further comprising a copolymer with electrostatic and steric mediating properties in a dose sufficient to decrease said adverse events in said subject relative to administration of said formulation lacking said copolymer with electrostatic and steric mediating properties wherein the said copolymer is selected from cationic graft, cationic block, hydrophobic graft, hydrophobic block, anionic graft, and anionic block copolymers; and the percentage of the copolymer solution is at minimum 0.01% by weight.

In one embodiment, the present invention provides a method to decease the adverse events associated with administration to a subject of antibiotic ophthalmic drug products selected from the group consisting of antibacterials, antifungals, antivirals, anti-helminthics, anti-parasitics, anti-acanthomoeba agents, and antiseptics, the method comprising administering said antimicrobial ophthalmic drug products in a formulation further comprising a copolymer with electrostatic and steric mediating properties in a dose sufficient to decrease said adverse events in said subject relative to administration of said formulation lacking said copolymer with electrostatic and steric mediating properties wherein the said copolymer is selected from cationic graft, cationic block, hydrophobic graft, hydrophobic block, anionic graft, and anionic block copolymers; and the percentage of the copolymer solution is at maximum 40% by weight for solutions and suspensions.

In one embodiment, the present invention provides a method to decrease the adverse events associated with administration to a subject an antibiotic ophthalmic drug product selected from the group consisting of an antibacterial, an antifungal, an antiviral, an anti-helminthic, an anti-parasitic, an anti-acanthomoeba agent, and an antiseptic, the method comprising administering said antimicrobial ophthalmic drug products in a formulation further comprising a copolymer with electrostatic and steric mediating properties in a dose sufficient to decrease said adverse events in said subject relative to administration of said formulation lacking said copolymer with electrostatic and steric mediating properties wherein the said copolymer is PLL-g-PEG.

In one embodiment, the present invention provides a method to decrease the adverse events associated with administration to a subject of an antibiotic ophthalmic drug product selected from the group consisting of an antibacterial, an antifungal, an antiviral, an anti-helminthic, an anti-parasitic, an anti-acanthomoeba agent, and an antiseptic, the method comprising administering said antimicrobial ophthalmic drug product in a formulation further comprising a copolymer with electrostatic and steric mediating properties in a dose sufficient to decrease said adverse events in said subject relative to administration of said formulation lacking said copolymer with electrostatic and steric mediating properties wherein the said copolymer is the copolymer is selected from the list of combinations described in the application supra.

A method for improving signs and symptoms of ocular adverse events associated with the chronic use of topical ophthalmic drug products comprising administering to a subject a said topical ophthalmic drug product further comprising a copolymer comprising bioadhesive and passivation moieties, where administration of said topical ophthalmic drug product further comprising said copolymer provides more protection of corneal and conjunctival epithelial cells from the toxicities APIs can cause with chronic use than said topical ophthalmic drug product without said copolymer.

In one embodiment, the present invention provides a method to reduce preservative interaction and the subsequent ocular adverse events associated with preserved topical ophthalmic therapy on corneal epithelial cells of a subject by exposing said cells to a copolymer with bioadhesive and passivation moieties in a manner selected from the group consisting of: a co-formulation, a preserved topical ophthalmic therapy delivered prior to exposure of said cells to said copolymer and a preserved topical ophthalmic therapy given after exposure of said cells to said copolymer, wherein said exposure of said cells to said copolymer provides a reduction in said preservative interaction and/or said subsequent ocular adverse events relative to that of said corneal epithelial cells exposed to a preserved topical ophthalmic but not exposed to said copolymer with bioadhesive and passivation moieties. In another embodiment, the present invention provides a method to reduce preservative interaction and the subsequent ocular adverse events associated with preserved topical ophthalmic therapy on corneal epithelial cells of a subject by exposing said cells to a copolymer with bioadhesive and passivation moieties in a manner selected from the group consisting of: a co-formulation, a preserved topical ophthalmic therapy delivered prior to exposure of said cells to said copolymer and a preserved topical ophthalmic therapy given after exposure of said cells to said copolymer, wherein said exposure of said cells to said copolymer provides a reduction in said preservative interaction and/or said subsequent ocular adverse events relative to that of said corneal epithelial cells exposed to a preserved topical ophthalmic but not exposed to said copolymer with bioadhesive and passivation moieties wherein the copolymer is PLL-g-PEG.

In one embodiment, the present invention provides a method to method to reduce adverse effects on epithelial cells of a subject, wherein said epithelial cells comprise corneal and/or conjunctival epithelial cells, wherein said adverse effects are caused by APIs in a topical ophthalmic drug product, said method comprising exposing said cells of said subject to a copolymer with bioadhesive and passivation moieties for a sufficient time and in an amount effective to reduce said adverse effects on said epithelial cells of said subject relative to that of said epithelial cells not exposed to said copolymer with bioadhesive and passivation moieties, wherein said exposure of said cells to said copolymer with bioadhesive and passivation moieties is selected from the group consisting of: a co-formulation comprising said copolymer with bioadhesive and passivation moieties and said APIs in said topical ophthalmic drug product, a topical therapy delivered prior to exposure to said APIs in said topical ophthalmic drug product and a topical therapy given after exposure to said APIs in said topical ophthalmic drug product. In another embodiment, the present invention provides a method to method to reduce adverse effects on epithelial cells of a subject, wherein said epithelial cells comprise corneal and/or conjunctival epithelial cells, wherein said adverse effects are caused by APIs in a topical ophthalmic drug product, said method comprising exposing said cells of said subject to a copolymer with bioadhesive and passivation moieties for a sufficient time and in an amount effective to reduce said adverse effects on said epithelial cells of said subject relative to that of said epithelial cells not exposed to said copolymer with bioadhesive and passivation moieties, wherein said exposure of said cells to said copolymer with bioadhesive and passivation moieties is selected from the group consisting of: a co-formulation comprising said copolymer with bioadhesive and passivation moieties and said APIs in said topical ophthalmic drug product, a topical therapy delivered prior to exposure to said APIs in said topical ophthalmic drug product and a topical therapy given after exposure to said APIs in said topical ophthalmic drug product, wherein the copolymer is PLL-g-PEG.

In one embodiment, the present invention provides a method to reduce in a subject ADC uptake by macropinocytosis into a cell selected from the group consisting of one or more of limbal stem cells, limbal epithelial cells, limbal epithelial daughter cells, transient amplifying cells, basal epithelial cells, wing cells, corneal epithelial cells, and differentiated corneal epithelial cells, said method comprising exposing said cells to a formulation comprising a copolymer with bioadhesive and passivation moieties prior to or subsequent to exposure in said subject to the ADC, wherein said formulation comprises a copolymer with bioadhesive and passivation moieties having a molecular weight (MW) that is both effective and safe and has a mean selected from the group of MWs consisting of: a MW<100 kDa, a MW<70 kDa, a MW<50 kDa, a MW<25 kDa, a MW<10 kDa, a MW<5 kDa, a MW<1 kDa, wherein said exposure provides a reduction in said subject of ADC uptake by micropinocytosis into said cell relative to that of said cells not exposed to said copolymer with bioadhesive and passivation moieties. In another embodiment, the present invention provides a method to reduce in a subject ADC uptake by macropinocytosis into a cell selected from the group consisting of one or more of limbal stem cells, limbal epithelial cells, limbal epithelial daughter cells, transient amplifying cells, basal epithelial cells, wing cells, corneal epithelial cells, and differentiated corneal epithelial cells, said method comprising exposing said cells to a formulation comprising a copolymer with bioadhesive and passivation moieties prior to or subsequent to exposure in said subject to the ADC, wherein said formulation comprises a copolymer with bioadhesive and passivation moieties having a molecular weight (MW) that is both effective and safe and has a mean selected from the group of MWs consisting of: a MW<100 kDa, a MW<70 kDa, a MW<50 kDa, a MW<25 kDa, a MW<10 kDa, a MW<5 kDa, a MW<1 kDa, wherein said exposure provides a reduction in said subject of ADC uptake by micropinocytosis into said cell relative to that of said cells not exposed to said copolymer with bioadhesive and passivation moieties, wherein the copolymer is PLL-g-PEG.

In one embodiment, the present invention provides a method to reduce ocular adverse events associated with an antibody-drug conjugate (ADC), the method comprising administering to a patient undergoing ADC treatment a formulation comprising a copolymer with bioadhesive and passivation moieties in an amount to be efficacious with at least one further agent of said formulation, wherein said agent improves the exposure of said copolymer to cells that would benefit from exposure to such copolymer and wherein said agent has one or more attributes selected from the group consisting of: an attribute of increasing mucous penetration, an attribute that increases tissue penetration and an attribute that increases corneal residence time. In another embodiment, the present invention provides a method to reduce ocular adverse events associated with an antibody-drug conjugate (ADC), the method comprising administering to a patient undergoing ADC treatment a formulation comprising a copolymer with bioadhesive and passivation moieties in an amount to be efficacious with at least one further agent of said formulation, wherein said agent improves the exposure of said copolymer to cells that would benefit from exposure to such copolymer and wherein said agent has one or more attributes selected from the group consisting of: an attribute of increasing mucous penetration, an attribute that increases tissue penetration and an attribute that increases corneal residence time; wherein the copolymer is PLL-g-PEG.

Methods for treating various aliments are described herein; certain embodiments of the present invention provide methods wherein said adverse event is selected from one or more of the group consisting of ocular discomfort, ocular irritation, decreased vision, dry eye symptoms, ocular hyperemia, conjunctival hyperemia, corneal staining, superficial punctate keratopathy, MECs, and refractive error changes.

In one embodiment, the present invention provides a preserved topical ophthalmic drug product that contains a preservative is selected from the group consisting of a as Polixetonium, polyquaternium-42, Polyquaternium-1, Polyquat, Alkyl-hydroxy benzoate preservatives, parabens, hydrogen peroxide, benzalkonium chloride (BAK or BAC), cetylpyidimine chloride, cetalkonium chloride, sodium perborate, Purite, disappearing preservatives, Polyhexamethylene biguanide (PHMB), chlorobutanol, Benzododecinium bromide, “Ionic buffered systems”, povidone, silver, silver sulfate, betadine, and other antiseptics and proprietary and non-proprietary preservatives.

In one embodiment, the present invention provides a topical ophthalmic drug product formulation further comprising a mucous penetrating technologies listed supra.

In one embodiment, the present invention provides a method to decease the adverse events associated with antibody-drug conjugate usage that is causing damage to (adversely affecting) nonneoplastic cells, by applying an effective amount of a copolymer with electrostatic and steric mediating properties to such cells and tissues (those that are adversely affected by antibody-drug conjugate usage); and the application of an effective amount of macropinocytosis inhibitor. In another embodiment, the present invention provides a method to decease the adverse events associated with antibody-drug conjugate usage that is causing damage to (adversely affecting) nonneoplastic cells, by applying an effective amount of a copolymer with electrostatic and steric mediating properties to such cells and tissues (those that are adversely affected by antibody-drug conjugate usage); and the application of an effective amount of macropinocytosis inhibitor, wherein the macropinocytosis inhibitor is described herein.

In another embodiment, the present invention provides a method to decease the adverse events associated with antibody-drug conjugate usage that is causing damage to (adversely affecting) nonneoplastic cells, by applying an effective amount of a copolymer with electrostatic and steric mediating properties to such cells and tissues (those that are adversely affected by antibody-drug conjugate usage) wherein the copolymer is PLL-g-PEG; and the application of an effective amount of macropinocytosis inhibitor, wherein the macropinocytosis inhibitor is described herein.

For coformulation issues, the components, or ingredients, in a coformulation may include: a multifunctional graft copolymer, a block copolymer, water, acids, bases, HCL, NaOH, mannitol, sodium phosphate, PEG, propylene glycol, glycerin, other demulcents, amphiphilic agents, magnesium chloride, potassium chloride, purified water, sodium chloride, zinc chloride. hydrochloric acid and/or sodium hydroxide, emulsifiers, solubilizers, other active agents, mucopenetration enhancers, mucopenetration technologies. Preservatives are included in some embodiments as well. Use in preservative free multidose bottles is contemplated. This section is not limiting, only providing some examples of formulations that may be used in embodiments described herein.

Concentration of mucopenetration technologies may range from very low (0.0001%) by weight to quite high (50%) by weight. For the purposes of specifications of mucopenetration technologies, any sub range of the concetration within 0.0001% and 50% may be delineated and is contemplated. For other actives, there may be more than one other active as well. The other active(s) may be formulated in the range between 0.0001% (for very small and/or typically toxic molecules) to up to 25% for safer molecules. For the purposes of specifications of other actives to protect corneal cells or actives that confer other ophthalmic benefits, any sub range of the concentration within 0.0001% and 50% may be delineated and is contemplated. pH ranges between 3 and 9 are contemplated with a preferred range 4.5% to 8.5%. Viscosity in some embodiments is between 2 centipoise and 20 centipoise (cP). Osmolarity is often between 140 milliosmoles (mosm) and 500 mosm. The preferred osmolarity is between 160 mosm and 350 mosm. Embodiments also include the sequential use of actives to protect corneal nerves and epithelial cells or limbal stem cells or their daughter cells (either administered prior to or after multifunctional graft copolymer). Sequential use means at least as frequently as using ach therapeutic during the same cycle of therapy which for example in ADCs is 21 days, or within 7 days prior to a cycle or for 56 days after a cycle as healing in these cases can be slow. The use of multifunctional graft copolymers to treat persistent corneal epithelial defects is an embodiment. The use of a multifunctional graft copolymer or a coformulation (other active or mucopenetration enhancers) with bandage contact lens therapy concomitantly or sequentially is an embodiment. Contact lenses in the current embodiment may serve as multifunctional graft copolymer reservoir for extended-release formulations. Extended release means a method or technology to increase the duration of delivery of a multifunctional graft copolymer (or an active agent that is not a multifunctional graft copolymer) to tissue compared with the pharmacokinetics or pharmacodynamics that is seen in drug delivery system not utilizing said method or technology. The multifunctional graft copolymer may be inside the polymeric component of the contact lens, in a contact lens solution, infused at a high concentration, actually formed as a contact lens itself for bandage purposes, or used as a drop on the lens or on the eye concomitantly. In this setting a range for concomitantly is: application to the eye within 24 hours before a contact lens is placed, within 72 hours after a contact lens is placed. Application to the contact lens can be performed during manufacturing or manually any time prior to contact lens insertion in the eye. An ophthalmic implant (a device that serves as a reservoir and delivery system) placed on the eye, under the conjunctiva, in the fornix, on the sclera, or locally, generally with regard to the eye being treated, as a method to deliver a multifunctional graft-copolymer, a macropinocytosis inhibitor, a neuroprotective agent protecting corneal nerves, or in any combination with other actives is an embodiment of this set of inventions.

For purposes herein oncology therapy, oncologic therapy, cancer therapy and autoimmune treatments may refer to a set of small molecules, biologics, CAR-T therapy, ADCs, stem cell therapy, and gene therapies aimed at treatments that treat and control neoplasia and or a hyper autoimmune response as may be seen in some inflammatory disorders. Oncology therapy may include agents (including biologics, or small molecules), immunotherapy (CAR-T) or bone marrow or stem cell transplantation; many are associated with corneal or conjunctival toxicity.

These multifunctional graft copolymer formulations can confer benefit to the conjunctiva, corneal surface, and corneal nerves for other drug products with ocular surface adverse events as well, including but not limited to topical glaucoma therapies and chronic ophthalmic treatments. For example, the chronic use of glaucoma medications can lead to subbasal plexus nerve changes, and the treatment with a multifunctional graft copolymer is an embodiment. There are multiple drugs that can cause corneal nerve toxicity, some, but not all, are oncologic agents. Other oncologic agents include, but are not limited to: bortezomib, paclitaxel, and oxaliplatin. Other biologics used in targeted therapies including monoclonal antibody approaches may lead to corneal nerve changes, indirectly. Intervention with the topical multifunctional graft copolymer may be as a preventative agent or to help treat and mitigate such corneal nerve damage once treatment with said oncologic agent has already been initiated. Imminent means the patient will be receiving such medication within 24 hours and 4 weeks. Prevention can start within this window, or even further in advance at the preference of the treating physician. Corneal nerve cells are considered at risk if a therapeutic agent associated with corneal nerve toxicity is planned to be used in said patient within days to months. In some patients there will already be underlying nerve damage due to other exposures, cancer, or ocular disease. In such situations the benefit is relative to the starting point of the subbasal plexus anatomy and morphology and can be compared to the alternative case where no multifunctional graft copolymer is or would be used in that setting. Benefits can also be seen in corneal and conjunctival epithelial infections whereby multifunctional graft copolymers limit tissue damage from toxins released in association with infection.

Another embodiment of this invention is that other actives with therapeutic benefit in corneal surface and nerve toxicities can be prepared in formulations alone (only active) without the requirement a multifunctional graft copolymer be present in the formulation. Mitigation means the action of reducing the severity, seriousness, or painfulness of a corneal toxicity. Mitigation can include prevention, pretreatment, concurrent treatment, cure, symptomatic relief, and/or management of a condition or side effect or finding or sign or symptom. Mucopenetration enhancing technology can include electrical stimulation of tissue and/or iontophoresis. Mucopenetration technologies can be used prior to the delivery of a multifunctional graft copolymer drug product. Other active can be delivered in different formulations but concurrently. Pretreatment can range from 7 days prior to 5 seconds prior. Preferred pretreatment time ranges are between 2 days prior and 5 seconds prior to a multidrug. Some mucopenetration enhancement technologies van also be delivered within 24 hours of treatment with the multifunctional graft copolymer. Concomitantly can mean at the same time; simultaneously. In this setting, however, concomitantly can mean at the same time that an ADC is in the systemic circulation. ADC's have half-lives of greater than a week. Belnatomab mafodotin for example, has a half-life of 12 days after the first infusoipn and 14 days in steady state. It can take 70 days to reach steady state. 5 half-lives until full clearance may be 70 days as well. Thus concomitantly (whether in the clinical, preclinical, or cell culture environment) means while the ADC is on board. Although two agents may be given within minutes or even hours and can be considered concomitant therapy, in the setting of ADC onboard (or other biologic or cancer therapeutic) several weeks can also be considered concomitantly as both agents are utilized while there is a drug with toxicity on board. An embodiment is treating corneal epithelial cells with both a multifunctional graft copolymer and a macropinocytosis inhibitor concomitantly to help protect said cells from ADC induced cell toxicity.

Concomitantly in this embodiment of dual treatment with multifunctional graft copolymers and another active (for example, as cytoprotectant, macropinocytosis inhibitor, cell stabilizer, growth factor, secretome) need not be delivered at the identical point in time, and it can mean only that the drugs are used together while there is ADC exposure to at risk cells. Neuroprotectant means an effect that may result in salvage, recovery or regeneration of the nervous system, its cells, structure and function. There are many neurochemical modulators of nervous system damage including those that protect nerve cells from dying and growth factors that promote growth and survival including nerve growth factors. Nerve growth factors may be recombinant or cell derived. Cytoprotection means helping cells live, function, and survive. Growth factors, molecule that limit toxic agent entry into cells, and molecules than confer cell survival can be used for cytoprotection. An embodiment is a method to reduce ocular toxicity due to systemic exposure of a human to ADCs with tubulin disruptors as the payload by treating the eye with an effective amount of cationic graft copolymer formulation and another agent that has a mechanism to confer additional benefit to corneal epithelial cells acting through mechanisms that confer therapeutic benefit. Surface of the eye can be considered the cornea and conjunctiva. In some embodiments of theses sets of inventions, coformulations may be considered combination drug products. In some embodiments, ADC therapy may encompass a set of toxicities that can overlap with adverse events associated with other biologics used to treat cancer, and multifunctional graft copolymers can be used in these settings effectively as well. In other embodiments, due to regulatory language, they may be formulations including several ingredients as described herein. Any formulation contemplated herein may be preserved or unpreserved. Coformulations may be unpreserved.

As described in Lin, Hui-Ping, et al. “Identification of novel macropinocytosis inhibitors using a rational screen of Food and Drug Administration-approved drugs.” British Journal of Pharmacology 175.18 (2018): 3640-3655, there are a set of clinically relevant agents that can inhibit macropinocytosis. It is an embodiment of this invention to utilize macropinocytosis inhibitors and other actives in addition to or concomitantly with multifunctional graft copolymers (sequentially or in the same formulation or in a kit) to mitigate, reduce, prevent or treat corneal epithelial toxicity and corneal nerve toxicity including the signs and symptoms associated with this adverse drug induced event. Although these pinocytosis inhibitors have been described a topical ophthalmic formulation or coformulation addressing ophthalmic toxicity is novel. These macropinocytosis inhibitor actives can be selected from lists below: Flubendazole and mebendazoles, Terfenadine and other H-1 receptor antagonists, Itraconazole and antifungals and triazoles, Phenoxybenzamine and Phenoxybenzamine, Vinblastine and vinca alkaloids, auranofin and disease-modifying antirheumatic drugs including gold Compounds, imipramine and protein kinase C PKC inhibitors, nystatin and polyenes, cytochalasion, chlopromazine and other antipsychotics, actin polymerization inhibitors, inhibitors of sodium/hydrogen exchangers. The actin perturbant cytochalasin D and latrunculins have been shown to inhibit membrane ruffling and macropinocytosis. The PI3K inhibitor wortmannin and LY290042 block macropinocytosis and phagocytosis in macrophages, fibroblasts and epithelial cells. Other macropinoctotisis inhibitors include: bifinazole, buspirone, quetiapine, tranycycromine, phorbol 12-myristate 13-acetate. Other embodiments include actives agents selected from the groups below.

The epithelial sodium channel blocker amiloride inhibits both constitutive and stimulated macropinocytosis and also phagocytosis in a variety of mammalian cells. The selective sodium hydrogen exchanger (NHE) blocker ethyl-isopropyl amiloride (EIPA) and dimethyl amiloride inhibit macropinocytosis. Other drugs included in this invention are those that inhibit clathrin-mediated and caveolin-mediated endocytosis. Genistein and methy-beta-cyclodextrin for example is an active included as an embodiment.

Endocytosis inhibitors and the class of each compound listed herein: hypertonic sucrose (sugars), potassium depletion (electrolytes), Cytosol acidification, Chlorpromazine, Monodansylcadaverine, Phenylarsine oxide, Chloroquine, Monensin, Phenothiazines, Methyl-β-cyclodextrin, Filipin, Cytochalasin D, latrunculin, Amiloride, Dynasore, Dynoles, dyngoes, Pitstop 2.

Endocytosis inhibitors delivered locally to the cornea are an embodiment of this invention to mitigate corneal toxicity related to ADCs and biologics, as are macropinocytosis inhibitors, mast cell stabilizers, antihistamines, PPAR agonsist, and classes of molecules listed herein. PPAR can have ebenficial effecst on epithelial cell preservation and nerve cell survival independent of any alteration of macropinocytotis activity. The use of such active agents in combination (as well as independently) is an embodiment of this invention. The use of any of these APIs alone or in combination with a multifunctional graft copolymer in sequence or in a coformulation is an embodiment of this invention.

PPAR agonists should be protective against ADC corneal toxicity and also help protect corneal nerve function. PPAR agonists include alpha and gamma among others. PPAR agonists can be used alone or in coformulations. Coformulation of fenofibrate and multifunctional graft copolymers for local ophthalmic delivery should be protective of the cornea and the subbasal corneal nerves. PPAR agonists can be formulated with a multifunctional graft copolymer. Fenofibrate alone should also be beneficial in the setting of corneal nerve damage associated with ADCs and is an embodiment.

Mast cell stabilizers: B2-adrenergic agonists, Cromoglicic acid, Ketotifen, Loratadine, Desloratadine, Methylxanthines, Olopatadine, Rupatadine, Mepolizumab, Omalizumab, Pemirolast, Quercetin, Nedocromil, Azelastine, Tranilast, Palmitoylethanolamide, Vitamin D[3],

Toxic molecules delivered locally to the cornea may prove effective for the eye, especially because ein the presence of a multifunctional graft copolymer synergistic benefits can be manifest with lower doses.

PKC inhibitors identified: soquinolines, H-7. Benzophenones, Chelerythrine, Balanol, Indolocarbazoles, Go6976, Go6983, Enzastaurin (LY317615), LY379196, Staurosporine, (CGP41251), CGP53353, UCN-01, Sotrastaurin (AEB071), Staurosporine analogs, Ruboxistaurin (LY333531), Midostaurin (PKC412, CGP41251), Bisindolylmaleimide (GF 109203X, Gö 6850), Ro 31-8220, SCH47112, Dicationic, lipophilic compounds, dequalinium C1, Flavonoid, Myricitrin, Quercetin, Benzothiazole riluzole, Perylenequinone, Calphostin C, (UCN-1028C), Phenolic ketone, Rottlerin (Mallotoxin), Macrolactone Bryostatin 1, (NSC 339555), Membrane lipids Sphingosine (d-erythro-Sphingosine), N,N-dimethyl-d-erythro-sphingosine, Taxol, Tamoxifen, Purine nucleoside Sangivamycin, Carbonitrile, 5-Vinyl-3-pyridinecarbonitriles, Pyrimidine, 2,4-Diamino-5-nitropyrimidine, Sterols, Spheciosterol sulfate A, B or C, Antisense oligonucleotides Isis3521 (CGP64128A, Aprinocarsen), Isis9606, Short peptides: Myristoylated-pseudosubstrate peptide inhibitor, αV5-3, βIV5-3, βIIV5-3, βC2-4, δV1-1 (KAI-9803, Delcasertib), ϵV1-2 (KAI-1678), KCe-12 and KCe-16, ZIP, γV5-3, α-tocopherol, adriamycin, aminoacridine, apigenin, cercosporin, chlorpromazine, dexniguldipine, polymixin B, trifluoperazine, UCN-02.

Antihistamines: azelastine, brompheniramine, cetirizine, chlorpheniramine, desloratadine, diphenhydramine, tetrahydrozoline/zinc sulfate, olopatadine, naphazoline/pheniramine, Alcaftadine, bepotastine, ketotifen, tetrahydrozoline/zinc sulfate, naphazoline/pheniramine, epinastine, phenylephrine, azelastine, pemirolast, tetrahydrozoline, oxymetazoline, naphazoline/zinc sulfate, nedocromil, cetirizine, levocabastine, emedastine, cromolyn, lodoxamide.

Topical ophthalmic steroids may have utility herein. Combinations may also include nerve growth factors in combination with multifunctional graft copolymers.

Without being bound by any particular theory, the use of some of the actives for inhibition of macropinocytosis or other methods to protect epithelial cell survival in the setting of corneal toxicity can be used in combination with multifunctional graft copolymers. One benefit of this approach is that very low and safe concentrations of these other actives can be beneficial in the presence of the multifunctional graft copolymer.

It has been shown in several clinical reports that corneal nerves, especially at the level of the subbasal nerve plexus can be reduced or damaged in ADC corneal toxicity and in corneal toxicity. Other oncological therapies can also cause a corneal neuropathy in addition to a peripheral neuropathy. Corneal nerve toxicity can exacerbate an epitheliopathy and delay healing. Not being bound by any particular theory, the epithelium supports the health of the corneal nerves and in turn the corneal nerves support the health of the corneal epithelial cells. Damaged epithelial tissue thereby may lead to indirect damage to the corneal nerves through loss of trophic and cell signaling support. The payload of an ADC may also be cleaved free within cells thereby releasing this toxic payload locally that may damage nerves. Or, the ADC may enter the nerve cells directly. Multifunctional graft copolymers may confer benefit in this setting in more than one manner. By reducing the uptake of ADCs into corneal epithelial cells, limbal stem cell daughter cells, (and possibly limbal stem cells themselves) as well as transient amplifying cells, wing cells, basal epithelial cells, epithelial cells and superficial epithelial cells, the downstream damage to subbasal corneal nerves and more superficial sensory nerve terminals can be reduced. The release of the payload may be involved in such toxicity, and the embodiments to inhibit ADC uptake will reduce linkage cleavage. Also, the mechanism of multifunctional graft copolymer may reduce direct entry into the nerve cell by toxic molecules.

PPAR agonists, cytokines and/or their derivatives including biologics (large molecules produced produced using a living system, such as a microorganism, plant cell, or animal cell) may also protect nerve cells in this setting and help treat or prevent a corneal neuropathy. An embodiment of this invention is the use of multifunctional graft copolymers applied to the cornea to prevent, mitigate, treat, or help resolve a corneal nerve toxicity associated with local or systemic pharmaceutical or biological therapies used in oncology treatments as well as in the management of other systemic diseases. An embodiment of this invention is the coformulation of a multifunctional graft copolymer with another agent that helps reduce ADC uptake through macropinocytosis or endocytosis to help protect corneal nerves. An embodiment of this invention is the coformulation of a multifunctional graft copolymer with another agent that confers cell protection, cell membrane stabilization, cell signaling, biologics, cellular growth factors including nerve growth factors and biomolecules found in the secretome of mesenchymal cells, blood cell, amniotic tissues, epithelial cells, and other cell systems and cell signaling systems to help protect corneal nerves. This combination (coformulation drug product) may confer additional therapeutic benefit to a treatment strategy aimed to reduce corneal toxicity including corneal nerve toxicity in patients treated with ADCs or oncology therapies associated with corneal nerve toxicity. Corneal adverse events (adverse effects) associated with ADC toxicity include but are not limited to: dry eye symptoms, ocular irritation, photophobia, eye pain, blurred vision, decreased vision, trouble driving, trouble reading, change in refractive error, epitheliopathy, whorl keratopathy, keratopathy, superficial punctate keratopathy, corneal erosion, corneal ulcer, microcyst-like changes, pseudomicrocysts, changes in corneal epithelial thicknesses, subepithelial haze, corneal scarring, decreased corneal sensation, changes to the corneal nerves (loss, dropout, reduced fiber length, increased tortuosity), changes to the corneal nerves in the subbasal nerve plexus. Corneal adverse events can include but are not limited to damage, but damage is one component. On the eye, nonneoplastic cells under consideration are limbal stem cells, conjunctival epithelial cells, conjunctival stromal cells, transient amplifying cells, wing cells, basal epithelial cells, corneal epithelial cells, superficial corneal epithelial cells, corneal nerves. In some embodiments some of these cells, but not all are protected through the use of multifunctional graft copolymers or other active agents. For some ADCs, conjunctival cells are also damaged, and that damage is mitigated by multifunctional graft copolymer use. Local delivery includes topical, subconjunctival, and periocular treatments.

Mesenchymal stem cell secretome has been shown to benefit persistent corneal epithelial defects. The concept of using this approach to mitigate, treat, or prevent corneal nerve or corneal epithelial toxicity associated with ADCs and other biologics or oncology agents is an embodiment of this invention. Secretomes and similar cell derived products associated with cell signaling mechanisms can be used to treat mitigate or prevent ADC related therapy and corresponding nerve damage. KPI-012 is such a mesynchymal secretome product. Other cell derived products are an embodiment herein for the mitigation, prevention, and treatment of ADC induced corneal epithelial and corneal nerve toxicity.

Formulations using semiflourinated alkanes as methods to deliver active agents to the cornea to mitigate toxicity and to inhibit ADC interactions with epithelial sells are an embodiment of this invention.

Contact lenses treated with multifunctional graft copolymers as a method to treat corneal adverse events associated with ADC toxicity are an embodiment of this invention. Contact lenses can be coated with multifunctional graft copolymers including PLL-g-PEG, stored in solutions with such multifunctional graft copolymers or loaded with multifunctional graft copolymers. There can be a synergistic benefit to having a contact lens and a multifunctional graft copolymer. An active that inhibits macropinocytosis may be included.

Another embodiment of this invention is a method to reduce changes and variability to the epithelial thickness and to thus secondarily reduce changes to the refractive error of an eye in patients being treated with ADCs.

Key embodiments included herein are: 1) topical ophthalmic coformulations with multifunctional graft copolymers and active agents that also may protect corneal epithelial cells or active agents that may also inhibit macropinocytosis or active agents or products that may otherwise help protect corneal epithelial cells and corneal nerves in the setting of ADC or other forms of drug-induced corneal toxicity. 2) Coformulations of topical multifunctional graft copolymers and mucpenetration enhancing molecules or technologies such that the effect of the multifunctional graft copolymer can be enhanced by allowing it to get deeper into corneal or conjunctival tissue-by penetrating mucin layer, glycocalyx, mucopolysaccharides and mucous membranes. Thus the multifunctional graft copolymer, for example, PLL-g-PEG, can more efficiently find its way to deeper layers of the cornea or corneal epithelial cells, subbasal nerve cells, wing cell, basal cells, transient amplifying cells, and layers just below the superficial epithelium. This approach will be particularly helpful in settings where ADC/cell interactions are occurring deep to the very surface of the eye. Protecting the limbal stem cells and minimizing or reducing a limbal stem cell dysfunction type clinical picture is an embodiment of this invention. 3) coformulations with mucous penetration enhancers and other agents that could benefit the corneal cell and a multifunctional graft copolymer. Concentrations of each of the three components including a multifunctional graft copolymer and other actives that could help protect corneal cells and mucous penetration enhancers range from 0.0001% to 40% for each component. 4) protecting corneal nerves through the use of local or topical multifunctional graft copolymers on the eye.

These topical ophthalmic formulations may be preserved or unpreserved. Unpreserved formulations may be delivered through preservative free multidose eye drop bottle technologies as well as through unit dosers.

### REFERENCES

1. Lin, Hui-Ping, et al. “Identification of novel macropinocytosis inhibitors using a rational screen of Food and Drug Administration-approved drugs.” British journal of pharmacology 175.18 (2018): 3640-3655.
2. Matlock H G, Qiu F, Malechka V, Zhou K, Cheng R, Benyajati S, Whelchel A, Karamichos D, Ma J X. Pathogenic Role of PPARα Downregulation in Corneal Nerve Degeneration and Impaired Corneal Sensitivity in Diabetes. Diabetes. 2020 June;69(6): 1279-1291. doi: 10.2337/db19-0898. Epub 2020 Mar. 25. PMID: 32213513; PMCID: PMC7243299.
3. Nakamura, Yoshikuni, et al. “Functional role of PPARô in corneal epithelial wound healing.” The American journal of pathology 180.2 (2012): 583-598.
4. Dutta D, Donaldson JG. Search for inhibitors of endocytosis: Intended specificity and unintended consequences. Cell Logist. 2012 Oct. 1;2(4):203-208. doi: 10.4161/cl.23967. PMID: 23538558; PMCID: PMC3607622.
5. Aschauer, Julia, et al. “Corneal Toxicity Associated With Belantamab Mafodotin Is Not Restricted to the Epithelium: Neuropathy Studied With Confocal Microscopy.” American Journal of Ophthalmology 242 (2022): 116-124.
6. Marquant, Kevin, et al. “Corneal in vivo confocal microscopy to detect belantamab mafodotin-induced ocular toxicity early and adjust the dose accordingly: a case report.” Journal of Hematology & Oncology 14.1 (2021): 1-8.
7. Farooq, Asim V., et al. “Corneal epithelial findings in patients with multiple myeloma treated with antibody-drug conjugate belantamab mafodotin in the pivotal, randomized, DREAMM-2 study.” Ophthalmology and Therapy 9.4 (2020): 889-911.
8. Mencucci, Rita, et al. “Corneal Findings Associated to Belantamab-Mafodotin (Belamaf) Use in a Series of Patients Examined Longitudinally by Means of Advanced Corneal Imaging.” Journal of Clinical Medicine 11.10 (2022): 2884.
9. Chiang, Jeremy Chung Bo, et al. “Corneal nerve changes following treatment with neurotoxic anticancer drugs.” The Ocular Surface 21 (2021): 221-237.
10. Ringvold, Helene C., and Raouf A. Khalil. “Protein kinase C as regulator of vascular smooth muscle function and potential target in vascular disorders.” Advances in pharmacology 78 (2017): 203-301.
11. Lühmann, Tessa, et al. “Cellular uptake and intracellular pathways of PLL-g-PEG-DNA nanoparticles.” Bioconjugate chemistry 19.9 (2008): 1907-1916.
12. Kleinman, David, et al. “PLL-g-PEG inhibits antibody-drug conjugate uptake into human corneal epithelial cells in vitro.” Investigative Ophthalmology & Visual Science 63.7 (2022): 3239-A0274.
13. Ando R, Kase S, Ohashi T, Dong Z, Fukuhara J, Kanda A, Murata M, Noda K, Kitaichi N, Ishida S. Tissue factor expression in human pterygium. Mol Vis. 2011 Jan. 8; 17:63-9.
14. Takahashi, Ji., Nakamura, S., Onuma, I. et al. Synchronous intracellular delivery of EGFR-targeted antibody-drug conjugates by p38-mediated non-canonical endocytosis. Sci Rep 12, 11561 (2022).
15. Renukuntla, Jwala, et al. “Expression of Folate Receptor (FR-alpha) and Proton Coupled Folate Transporter (PCFT) on RPCEC and HCEC Cell Line.” Investigative Ophthalmology & Visual Science 59.9 (2018): 3852-3852.
16. Corbelli, Eleonora, et al. “Ocular toxicity of mirvetuximab.” Cornea 38.2 (2019): 229-232.

Throughout this application various publications both patent related and non-patent literature, are referenced. The disclosures of these publications in their entireties are hereby incorporated by reference into this application in order to more fully describe the state of the art to which this invention pertains.

### Examples

Results show significant benefit of the primary endpoint and for multiple secondary analyses.

### Example 1

Respiratory epithelial cells in culture and with the presence of a SARS-CoV-2 viral particles leads to very high infectivity rates of these cells and their subsequent demise. When a cationic graft co-polymer is added in solution to this system, at concentrations of 0.001, 0.01, 0.1, 1, 2, and 3% w/w for long enough to allow significant infection rates, for example hours. The viral infectivity is reduced and cell survival is enhanced, on a dose response basis. The negative control is solution only with lack of any cationic graft copolymer. There are multiple experiments performed to show this beneficial effect. One involves cell survival as measured with a cytometer, another involves quantitative PCR to measure the number of copies of the viral genome. In one experiment, viable respiratory epithelial cells are counted in solution to provide a #live respiratory epithelial cells/ml. PLL (20 kDa)-g[3.5]-PEG(5 kDa) is added as a lyophilized powder in different amounts to provide the concentrations tested into the various wells and cell cultures (standard growth and survival media). The same amount of SARS-COV-2 viral particles, at an absolute viral particle numbers per well, is added. After 24 hours cell viability is counted. Experimentation prior shows the best number of viral particles to add. Empiric data suggests 10,000 viral particles per ml is effective at infecting the respiratory cells with the majority of cultured cells in the well dying at 24 hours. The solutions containing PLL-g-PEG showed improved cell survival as shown below in Table 1.

TABLE 1

| | Respiratory cell survival | Conclusion |
|---|---|---|
| Concentration PLL-g-PEG | | |
| % on a w/w basis in | | |
| presence of SARS-Cov-2 | | |
| None (negative control) | lowest seen | Standard control |
| | | result |
| 0.001 | slightly improved vs. | Evidence of efficacy |
| | control | |
| 0.01 | improved vs. control | Effective |
| 0.1 | improved vs. control | Effective |
| 1 | improved vs. control | Effective |
| 3 | improved vs. control | Effective |

Another method to demonstrate the same effectiveness of the intervention with a cationic graft copolymer in the setting of SARS-Cov-2 infection is performed using a similar design, except the endpoint is RNA copies of the viral genome. In this experiment, the same PLL-g-PEG concentrations are used, but the cells are washed from all viral media and PLL-g-PEG media after exposure and a 2-hour incubation period. 24 hours later the solutions are tested for viral RNA copies. Detection of the SARS-COV-2 virus is performed using the One Step Prime Script RT-PCR kit on the Light Cycler 480 Real-Time PCR system with primers. The following sequences are used: forward primer: 5′-AGAAGATTGGTTAGATGATGATAGT-3′ (SEQ ID NO:1); reverse primer:5′-TTCCATCTCTAATTGAGGTTGAACC-3′ (SEQ ID NO:2); and probe:5′-FAM-TCCTCACTGCCGTCTTGTTG ACCA-BHQ1-3′ (SEQ ID NO:3). All experiments are conducted in triplicates. Table 2 shows these results.

TABLE 2

| | RNA viral copies | |
|---|---|---|
| | (qualitative) | Conclusion |
| Concentration PLL-g-PEG | | |
| % on a w/w basis in | | |
| presence of SARS-Cov-2, | | |
| prior to transition to pure | | |
| media | | |
| None (negative control) | Most | Standard control result |
| 0.001 | Fewer | Evidence of efficacy |
| 0.01 | Fewer | Effective |
| 0.1 | Fewer | Effective |
| 1 | Fewer | Effective |
| 3 | Significantly fewer | Effective |

These studies demonstrate statistical significance. This experiment reduces to practice the specific opportunity with the utilization of cationic graft copolymers to reduce SARS-COV-2 viral infectivity into at risk cells, and hence, tissues and organisms.

### Example 2

A clinical study is conducted to demonstrate the clinical benefit of this invention. Humans at risk for SARS-COV-2 viral infection (COVID19) are treated with topical formulations of PLL-g-PEG. These formulations are liquid based. A person at risk for SARS-COV-2 viral infection based on high-risk exposure through a workplace environment is enrolled in the trial. 1000 patients are enrolled in a 1:1:1 randomization schema. The study is double masked. Dose A is PLL-g-PEG as follows: eye drop 0.1%, Nasal spray 0.1%, and oral rinse 0.1%; Dose B is eye drop 0.5%, Nasal spray 0.5%, and oral rinse 0.5%. Cohort C receives saline only via eye drop, nasal spray and oral rinse. Participants are stratified equally by sex, age (≥60, <60) and as front-line emergency medical providers in acute care high volume COVID19 hospital settings or police officers and emergency medical technician first responders to medical calls. At enrollment all participants are Sars-Cov-2 negative and are provided with the solutions for use. Instructions are to apply to ocular, nasal, and oral mucosa just prior to entering the at-risk environment. Applications must be repeated every four hours as needed based on exposure risk. Standard precautions are utilized in addition to the PLL-g-PEG formulations or saline. Duration of study is two weeks of treatment or control. The endpoints assessed at 4 weeks include is number of A) infected study subjects following initiation of therapy or control and B) severity of illness on a grade of 1-resolved without hospitalization, 2-hospitalization required, 3-ICU required or death. As a substudy, swabbing demonstrated statistically equivalent amounts of viral particles on the patient's personal protective equipment and on inanimate object surfaces such as the counter and bedside table in the room. The person at risk for infection, practices personal safety precautions, but despite this, the exposure of the at-risk person's mucous membranes is significant. Infection of the at-risk person is prevented because the use of the cationic graft copolymer formulations, showing reduction in infection rates and disease severity.

Table 3 shows results.

TABLE 3

| | Infections | Mean severity |
|---|---|---|
| | COVID19 | of infections |
| Concentration PLL-g-PEG | | |
| % formulations on a w/w | | |
| basis in study participants | | |
| None (negative control) | many | most |
| 0.1 | fewer | reduced |
| 0.5 | fewer | reduced |
| 1 | Lower than 0.5% dose | reduced |
| | infection | |

### Example 3: ADC in vitro

ADC is AGS-16C3F: AGS-16C3F is an antibody-drug conjugate against ectonucleotide pyrophosphatase/phosphodiesterase 3 (ENPP3) containing the mcMMAF linker-payload for treatment of metastatic renal cell carcinoma. AGS-16C3F or Rituxumab-mcMMAF as a test article (an ADC that binds CD20) or other ADCs may be used. Other ADCs have been reported to cause ocular toxicity in patients by mechanisms for which understanding is just developing. This invention represents an approach based on experimentation and new ideas about this toxicity.

Human primary corneal epithelial cells are cultured in kertinocyte-SFM and HCEC cells from ATCC are cultured in corneal epithelial cell basal medium supplemented with corneal epithelial cell growth kit. Human cell lines are confirmed utilizing short tandem repeat profiling and confirmed to be Mycoplasma negative. Cells are seeded onto multiple 8-well chamber slides and cultured for 48 hours prior to treatment and subsequent immunostaining. Cells are treated with a negative control (no copolymer) and also simply PLL and PEG (not grafted together). The other cell cultures are exposed to PLL-g-PEG (PLL(20 kDa)-g[3.5]-PEG(5 kDa)) as prime example. Other PLL-g-PEGs are tested similarly. PLL-g-PEG concentrations are 0.001, 0.01, 0.1, 0.3, 0.5, and 1% w/w. Keratocytes are able to maintain viability after treatment with PLL-g-PEG, but in the setting of an in vitro experiment some keratocytes may die in the experiment. Keratocytes are then incubated with AGS-16C3F (or Rituxumab-mcMMAF or another ADC) with and without coincubation of 0.5 mg/mL Dextran-Texas Red for 4 hours at 37° C. Inhibition of macropinocytosis as a control is evaluated by treating cells with EIPA for 30 minutes prior to AGS-16C3F (or other ADC)/Dextran-Texas Red incubation. After the incubation period, unbound antibody and copolymer is washed off with PBS and cells are fixed in 4% paraformaldehyde for 20 minutes at room temperature. Cells are then permeabilized in PBS plus 0.1% Triton-X-100 for 15 minutes, and nonspecific labeling is blocked in PBS plus 10% normal goat serum. Cell surface-bound and internalized cytosolic AGS-16C3F (or other ADC) is visualized by incubating cells with Alexa Fluor 488-labeled goat anti-human IgG. Nuclei are visualized with TO-PRO-3 Iodide, and coverslips are mounted using ProLong Gold Antifade reagent for imaging. High-resolution laser confocal image sections are acquired using a Leica TCS SP5 II (63× oil immersion objective; NA ¼ 1.4) and are scanned sequentially to minimize fluorophore cross-talk and false-positive colocalization. ADCs (2 mg/mL or other concentrations) are prepared in PBS, and 1:2 serial dilutions are made in a black-walled, 96-well plate (21, 23). Equal volume of 1,8-ANS is added and incubated for 30 minutes at room temperature. Fluorescence signal is measured.

### Results:

Upon microscopy, many fewer stained ADCs entered PLL-g-PEG treated epithelial cells. A PLL-g-PEG concentration dose response was exhibited. See Table 4.

TABLE 4

| Concentration PLL-g-PEG | |
|---|---|
| % formulations on a w/w | Intracellular uptake by |
| basis in study arms | staining experiment |
| None (negative control) | High |
| None (PLL and PEG not | High |
| grafted together) | |
| 0.001 | Relatively less to no difference |
| 0.01 | Less than negative control |
| 0.1 | Less than negative control |
| 0.5 | Less than negative control |
| 1 | Significantly less than negative control |

Thus, PLL-g-PEG and by extension mimetics are an effective intervention for reducing ADC related corneal toxicity.

Experiments with ADCs and tubulin disruptors and other cytotoxins are carried out and this benefit is not limited to one type of ADC. In fact, any ADC with corneal epithelial toxicity is amenable to relief with this approach. This approach is not ADC dependent and is a method to reduce this class effect corneal toxicity.

The PLL-g-PEG can also be added slightly after the ADC is added to solution, so in some embodiments benefit remains if ADC exposure is prior to PLL-g-PEG exposure.

### Example 4: ADC In Vivo

ADC related corneal toxicity is reduced in in vivo models. Rabbits are commonly used to test possible drug-mediated eye toxicities and are chosen to investigate the ocular toxicity of ADCs. AGS-16C3F and other ADCs are useful in these experiments. In vivo animal toxicity, although very prevalent in the human, is somewhat variable as a model in the animal. The rabbit shows varying toxicity to various ADCs. For this experiment, multiple ocular studies testing AGS-16C3F (AGS-16C3F is an antibody-drug conjugate against ectonucleotide pyrophosphatase/phosphodiesterase 3 (ENPP3) containing the mcMMAF linker-payload for treatment of metastatic renal cell carcinoma) and other ADCs with tubulin inhibiting payloads are carried out in rabbits. Five animals in each group are dosed. There is a non-ADC dosed control arm that shows no toxicity in any anuAGS-16C3F at 10—and 15-mg/kg doses are used and show ADC toxicity. Rabbits dosed at 15 mg/kg show reversible conjunctival hyperemia, perilimbal corneal haze, corneal edema, and ciliary flush. Likewise, the 10-mg/kg once-weekly dose causes ophthalmologic toxicity as well. Versus no exposure to PLL-g-PEG via topical treatment at the initiation of ADC dosing, the rabbit eyes of those treated with topical PLL-g-PEG eye drops dosed three times daily at 0.5%, 1% and 2% show reduced toxicity in this experiment. On average the benefit is >5%, >7.5%, and >10% better in the PLL-g-PEG dosed groups, 0.5%, 1%, and 2%, respectively. Dosing is daily with the PLL-g-PEG eye drop and control. ADC infusion is weekly. A beneficial effect is also seen when the PLL-g-PEG is administered beginning one week after dosing with ADC, although the benefit is reduced by approximately 20% in each group at end of experiment. PLL-g-PEG treated animals have fewer ADC related toxicities versus control (artificial tears only). All formulations are preservative free. Preserved formulations are effective but have the unwanted effects of causing or exacerbating a corneal epitheliopathy, nevertheless, in a rabbit model benefit is shown even using preserved solutions.

Subconjunctival formulations are also protective when delivered subconjunctivally once weekly.

Intravenous formulations are safe and acceptable and efficacious; however, local delivery is more efficacious.

Results are documented through exam findings (including staining) and histopathology at Days 21 and 42 (fewer infiltrates and tissue damage).

The use of PLL-g-PEG solutions improves recovery time in treated animals.

Note that the rabbit findings do not replicate human pyknotic findings identically, but the model demonstrates anterior corneal abnormalities both clinically and on histopathology.

Sample ADCs selected, results are repeated with other ADCs with tubulin inhibitors.

See Table 5

TABLE 5

| Concentration % PLL-g-PEG | | |
|---|---|---|
| formulations on a w/w basis | | |
| in animal groups treated with | Examination | Inflammation by |
| AGS-163CF | findings | histopathology |
| None (negative control) | Significant corneal | Significant |
| | staining | inflammatory cells |
| 0.5 | Decreased staining | Reduced inflammation |
| 1 | Decreased staining | Reduced inflammation |
| 2 | Decreased staining | Reduced inflammation |

### Example 5: ADC in Humans

This study is run in patients being prescribed ADCs for oncologic disease using ADCs with known corneal toxicity. Commercially available ADCs are utilized in a clinical care setting for approved ADCs, as are patients receiving not yet approved ADCs under clinical investigations as part of the regulatory approval process. Thus, this randomized controlled trial is performed in patients on multiple different ADCs; stratification is by ADC and pre-existing eye discomfort.

75 patients are enrolled as it is determined empirically this total N with 25 subjects per arm is sufficient to detect a difference in eye findings.

All patients are enrolled at the initiation of therapy. Patients are randomized to preservative free artificial tears only, 1% PLL-g-PEG eye drops (unpreserved, and 2% PLL-g-PEG eye drops (unpreserved). All patients are instructed to use the drops in both eyes four times per day starting one day before Cycle 1 ADC dosing. Eye exams are performed at Baseline and at every Cycle through Cycle 4. One Cycle is 21 days.

After Cycle 4, patients are allowed to stay on PLL-g-PEG if they are enrolled into a treatment arm. Those randomized to artificial tears are allowed to cross-over to PLL-g-PEG eye drops.

The extent of corneal microcyst-like epitheliopathy or superficial punctate keratitis or keratopathy is assessed using an objective analysis estimating the density and region of the epitheliopathy and/or assessments of epithelial injury by staining with vital dyes such as fluorescein. A score based on percent of cornea involved multiplied by a correction factor of overall clinical severity (1 to 3) is used. The study endpoint is most severe score over study duration.

Results follow:

TABLE 6
Study results Clinical trial in reducing epithelial keratopathy
AEs ay worst time point

| Dose Group | Microcyst like score | LogMAR BCVA |
|---|---|---|
| Control | highest | impaired vision |
| 1% PLL-g-PEG | Lower | mildly less impaired |
| | | vision |
| 2% PLL-g-PEG | Lower | mildly less impaired |
| | | vision |

Adverse events of blurred vision and ocular irritation are highest in controls, and lower in low dose and high dose.

Dose holds and dose delays in each group is highest in controls, and lower in low dose and high dose.

PLL-g-PEG treated eyes show less corneal superficial punctate keratitis. Visual acuity is better, on average, in PLL-g-PEG (or copolymer) treated eyes and patients.

This experiment reveals, by example, the clinical value of this intervention with PLL-g-PEG solution used topically on the eye to reduce the adverse corneal effects of ADCs.

A concomitant intravenous delivery of a 1% PLL-g-PEG solution at each cycle of dosing shows less thrombocytopenia in treated subjects, thereby demonstrating the broader effect of mitigating ADC toxicity to systems that extend outside the corneal epithelium, thus supporting broader claims.

### Example 6

When a variety of novel ADCs are assessed preclinically for uptake into a human corneal epithelial cell in the presence of PLL-g-PEG formulations, then those specific ADCs that show greatest benefit (most reduced uptake) in the presence of PLL-g-PEG can be advanced into the clinic. One reason for a selection is there is now a known and effective clinical method to treat, mediate, mitigate, reduce, and prevent ADC toxicity in that cell type with co-treatment with copolymers claimed and discussed herein

Three variations of ADCs that target the cell receptor found in an oncologic disease with unmet need are assessed in the lab. All show significant macropinocytotic uptake into human corneal epithelial cells. PLL-g-PEG solutions of varying concentration (range 0.01 to 3% weight/weight are then used in a similar test of human epithelial cell ADC uptake in vitro. Assessments of the potential toxicity of ADCs is reassessed. PLL-g-PEG at 0.01% or higher (not necessarily inclusive) is found to significantly reduce the uptake of one of the three ADCs in vitro at a percentage that may vary based on ADC, but is still effective. This effect is confirmed in a rabbit in vivo model. The molecule is thus advanced to the clinic ahead of the others as there is a known method to minimize uptake with locally applied PLL-g-PEG. Other bioadhesive/passivation copolymers are used in ADC development in a similar manner as the experiments described above.

### Example 7

### ADC Uptake Experiment.

### Human Corneal Epithelial Cells

HCE-T cells (Araki-Sasaki et al. 1995) were obtained from RIKEN BioResource Center/RIKEN Cell Bank (RBRC-RCB2280, Lot 005, Tsukuba, Japan) and cultured according to RIKEN Cell Bank's instructions. Briefly, HCE-T cells were cultured in DMEM/F-12 medium supplemented with 5% fetal bovine serum, 1% Penicillin-Streptomycin, 5 μg/mL insulin, 10 ng/mL hEGF, 0.5% DMSO on T-25 cell culture flasks at 37° C. in 5% CO2/95% humidity.

All cell culture wells were coated with poly(L)lysine (not PLL-g-PEG) prior to all testing. Stock solution (0.1% w/v in sterile water) was prepared and filter sterilize through a 0.22 μm filter. Aliquots were stored at −20° C. When ready to use, stock solution was diluted 1:20 to prepare a 50 μg/mL working solution and cell culture wells or chambers were coated with the working solution and incubates 1 h in a 37° C. incubator. The solution was removed by vacuum aspiration and surface was allowed to dry. Coated cell culture ware was stored at 4° C. until use.

### Study Compounds

The test article (PLL-g-PEG polymer, 2.94% solution) was delivered and specific ADC (Rituxumab-mcMMAF, 3.3 mg/mL solution) was delivered.

Non-expression of rituximab target was demonstrated on human corneal epithelial cells to ensure that interaction for this experiment was not target driven.

Cells were shown to be viable when exposed to PLL-g-PEG.

The Inhibitory Concentration 50% (IC50) was found to be 33.8 nanomolar. The amount used at 4 hours was 75 nm as the IC50 was taken over 96 hours and uptake could be higher for a shorter 4-hour experiment.

Cellular ADC uptake assay was performed similarly as described in Zhao et al. (2018). Cells were seeded (40 000 cells/well) on PLL (not PLL-g-PEG) coated 8-well chamber slides and cultured for 48 h prior to treatment. Inhibition of macropinocytosis was evaluated by treating cells with PLL-g-PEG (0.0625%, 0.125%, 0.625% w/v) using water as vehicle (corresponding to the 0.625% teat article concentration) and 1.25% w/v test article with a matching water control; 5-(N-ethyl-N-isopropyl)amiloride (EIPA, 188 μM) was used as positive control in 400 μlL/well for 30 minutes prior to ADC/Dextran-Texas Red incubation. Tests were run in quadruplicates (four technical replicates).

After 30 min pre-incubation, ADC was administered by addition of 100 μL volume of media with 5× rituximab-MMAF (ADC, 375 nM). Thus, final concentrations during incubation were-20% of the original concentrations, i.e., PLL-g-PEG 0.05%, 0.1%, 0.5%, and 1.0% w/v; ADC 75 nM; EIPA 150 μM (as per Zhao et al. 2018). ADC will be administered with and without co-incubation of 0.5 mg/mL Dextran-Texas Red (10,000 MW, Thermo Fisher, D1863) for 3.5 h at 37° C. Following the incubation period, unbound antibody was washed off with DPBS and cells were fixed in 4% paraformaldehyde (PFA) for 20 minutes at room temperature. Cells were permeabilized 0.1% Triton X-100+10% goat serum in DPBS for 15 minutes, and nonspecific binding was blocked using 10% goat serum in DPBS. Cell surface-bound and internalized cytosolic ADC was detected by incubating cells with Alexa Fluor 488-labeled goat anti-human IgG (Thermo Fisher Scientific, catalog no. −11013) in Tris buffered saline at +4° C. overnight. Nuclei were visualized with 4′,6-Diamidino-2-phenylindole dihydrochloride (DAPI), and coverslips were mounted using Fluoroshield (Thermo Fisher Scientific) for imaging. ADC and dextran-Texas Red were visualized by high-resolution images acquired using a Leica THUNDER 3D Tissue Imager.

Each image workflow was optimized with random images as reference. Initial adjustment of Brightness/Contrast and thresholding remained consistent throughout all measurements within all experimental groups. This adjustment was made in order to decrease background noise and increase fluorescence intensity for clearer cell perimeters for the ImageJ software to register. Cells were mapped by ImageJ using DAPI stain, and the cellular outlines were overlayed on the green and red fluorescence images to quantify the amount of fluorescence within the cells. Also, full image fluorescence was measured to compare the amount of green fluorescence within the cell to determine the fluorescence differences in the intracellular and extracellular space. Below are representative images of the green fluorescence observed in groups of varying concentrations of PLL-g-PEG.

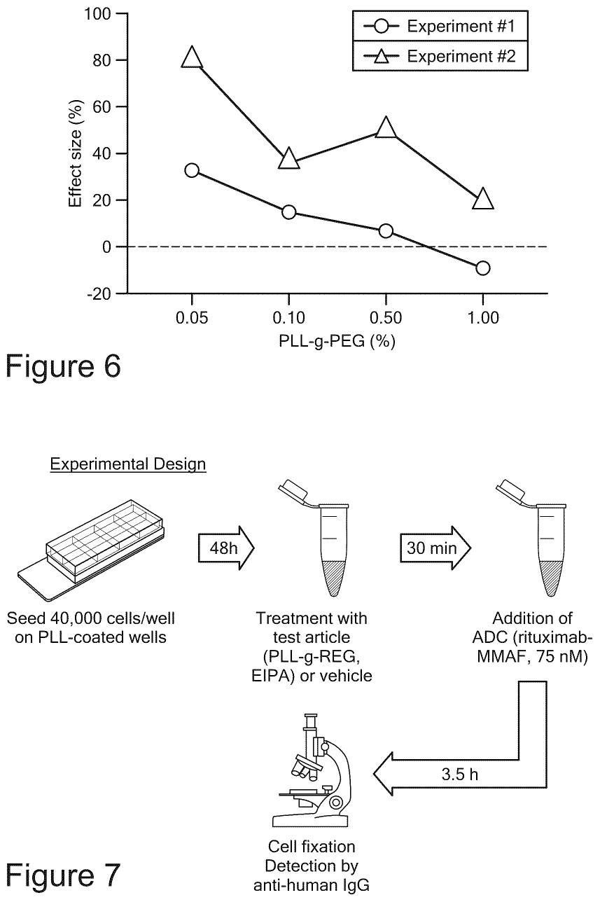

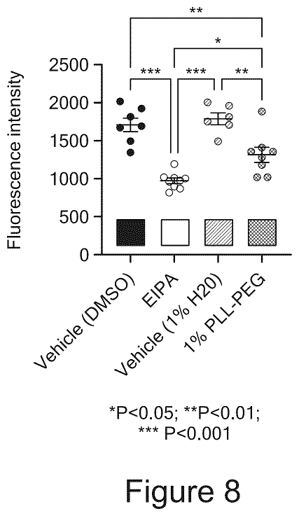

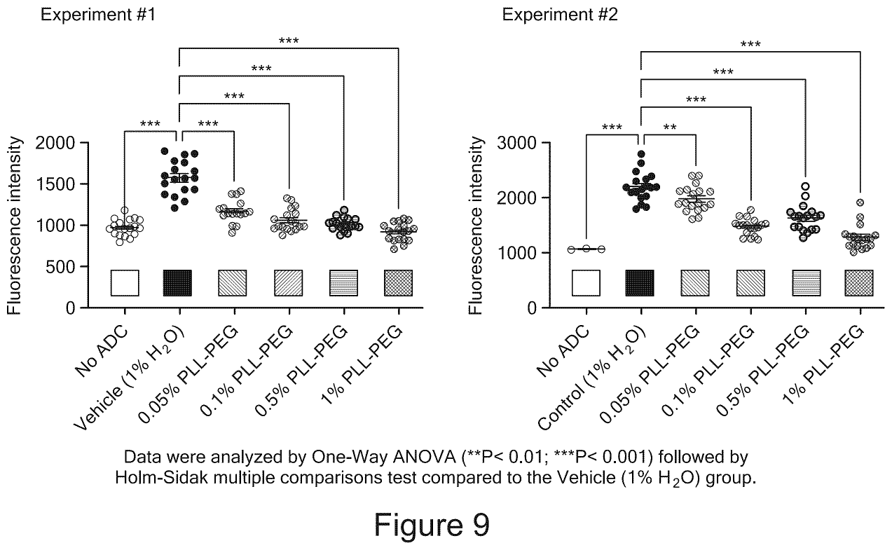

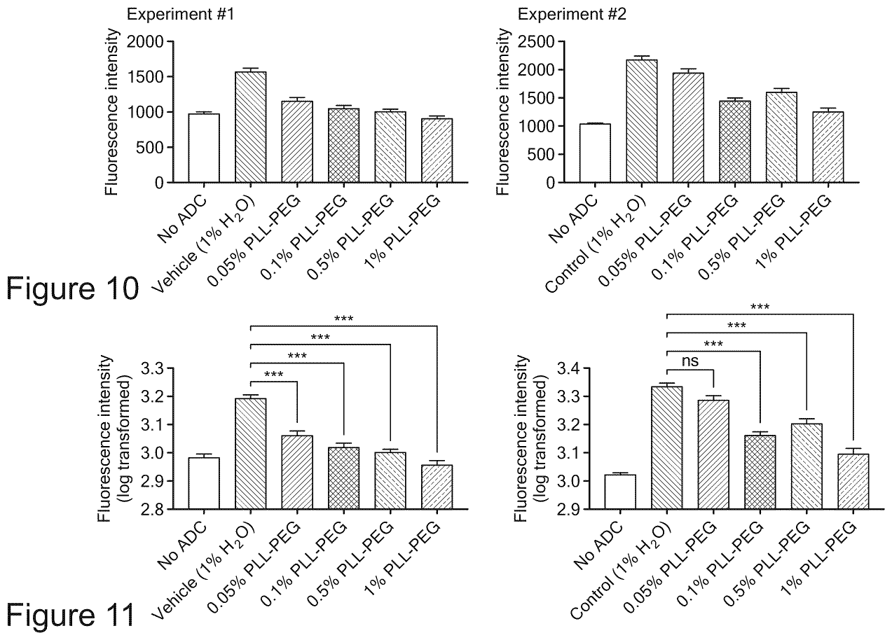

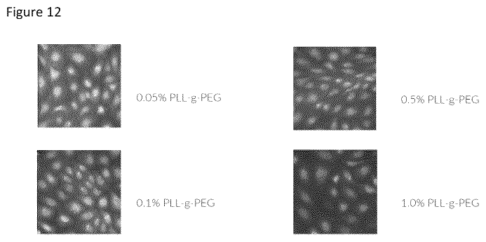

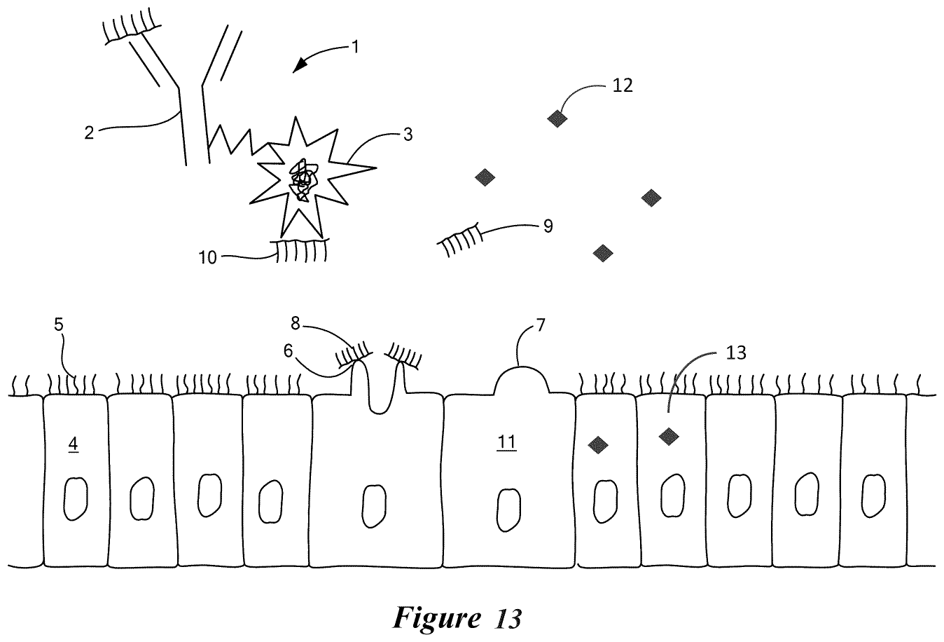

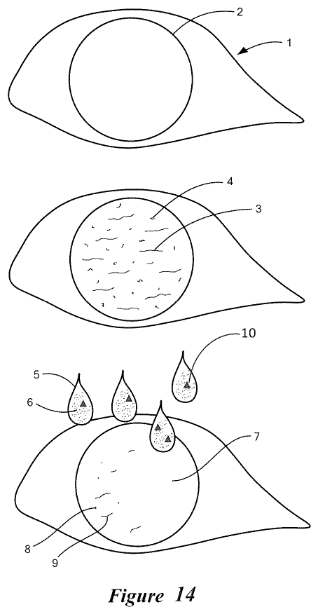

CONCLUSION: PLL-g-PEG showed a dose-dependent inhibition of ADC uptake into HCE-T cells, as evidenced by the data from FIGS. 6 through 12.

Note, other experiments are run such that interference and reduction of uptake and efficacy is demonstrated by PLL-g-PEG and for multifunctional graft copolymers for ADCs that also have on-target driven (receptor-driven) adverse effects on corneal and conjunctival cells as well.

### Example 8

A three-dimensional cell-based assay allowing for proliferation of cells in a suspension as opposed to surviving on a substrate is described whereby cell survival can be assessed independent of effects on the adherence of those cells to a substrate. In this model, the protective effect on corneal epithelial cells can be assessed on a cell-survival based assay measured over days.

### Example 9

A clinical experiment showing reduction of benzalkonium (BAK) related toxicity is carried out with the following results:

When PLL-g-PEG is formulated with BAK for topical ophthalmic use, patients tolerate the PLL-g-PEG formulation better than products with BAK but without PLL-g-PEG. Patients show decreased rates of medicamentosa (a chemical irritation of ocular and/or adnexal tissues by a topically applied drug) and signs of ocular irritation including conjunctival and corneal epithelial cell staining with rose bengal, fluorescein and lissamine green and fewer reported events of discomfort using the SANDE scale and the OSDI scale in PLL-g-PEG treated vs. control. The trial is 2 weeks in duration.

### Example 10

A clinical experiment showing reduction of antibiotic related toxicity is carried out with the following results:

When PLL-g-PEG is formulated with a bacitracin and polymyxin B sulfate antibiotic for topical ophthalmic use, patients tolerate the PLL-g-PEG formulation better than a topical ophthalmic drug product of these antibiotics in combination without PLL-g-PEG. Patients show decreased rates corneal toxicity and ocular discomfort in PLL-g-PEG treated vs. control. The trial is two weeks in duration.

### Example 11

A clinical experiment showing impressive efficacy (very good therapeutic response) with PLL-g-PEG and tissue or mucous penetrating technology is carried out in ADC-treated patients with the following results:

When PLL-g-PEG is formulated with a tissue and mucous penetrating technology for topical ophthalmic use, patients tolerate the therapy well and show reduced ADC-related corneal toxicity as assessed clinically and based on symptoms.

### Example 12

In patients undergoing ADC therapy for cancer, an active agent selected from the lists of pharmaceuticals supra (including but not limited to macropinocytosis inhibitors, neuroprotectants, pinocytosis inhibitors, antihistamines, mast cell stabilizers) in a topical ophthalmic formulation shows fewer adverse events and dose holds in the treated patients vs. the standard of care treated patients. This benefit is greater when coformulation with a multifunctional graft copolymer is carried out. Efficacy either active in this setting alone is also an embodiment of this invention, and shows superior efficacy compared to standard of care eye drops (currently artificial tears) or no treatment. There is synergy in such a combination product or coformulation when utilizing a multifunctional graft copolymer.

### Example 13

In patients undergoing ADC therapy for cancer, the use of a multifunctional graft copolymer topical ophthalmic solution (or delivered locally) shows that compared to standard of care treated controls fewer changes to the corneal subbasal nerve plexus by corneal confocal microscopy. These changes include greater density to nerves, longer nerve fiber length, and less tortuosity by CCM.

### Example 14

Coformulation with a mucopenetration enhancer improves the clinical benefit of topical PLL-g-PEG in patients on ADC therapy as PLL-g-PEG can get more effectively deeper into corneal tissue to inhibit macropinocytosis in cells deeper than the most superficial layers. For example, limbal stem and daughter cells gaining exposure to an ADC through the limbal circulation can be better protected from ADC-induced toxicity and cell damage.

### Example 15

A clinical experiment showing reduction of glaucoma related toxicity is carried out with the following results:

When PLL-g-PEG is formulated with an intraocular pressure lowering agent for topical ophthalmic use, patients tolerate the PLL-g-PEG formulation better than a topical ophthalmic drug product of these intraocular pressure lowering agents (used chronically) without PLL-g-PEG. Patients show decreased rates corneal toxicity and ocular discomfort in PLL-g-PEG treated vs. control. There is less damage to nerves in the subbasal plexus as well. The trial is 6 months in duration.

1. A method to treat one or more adverse events in a patient undergoing therapy comprising treatment with an antibody-drug conjugate, wherein said adverse events comprises damage to or adversely affects cells not specifically targeted by said antibody cells in said patient, the method comprising:
applying to said nontargeted cells or to tissues comprising said nontargeted cells of said patient a formulation comprising a multifunctional graft copolymer in an amount and for a time effective to decrease said one or more adverse events to said cells or to tissues comprising said cells of said patient,
wherein said decrease in said one or more adverse effects to said nontargeted cells or to tissues comprising said nontargeted cells of said patient is with respect to exposure by said nontargeted cells to said antibody-drug conjugate in the absence of said copolymer, wherein said copolymer displays electrostatic and steric mediating properties, wherein the copolymer is selected from the group consisting of a cationic graft, cationic block, hydrophobic graft, hydrophobic block, anionic graft, and an anionic block copolymer,
wherein optionally, the copolymer is formulated with a mucopenetrating agent and/or a macropinocytosis inhibitor,
wherein preferably, the copolymer is formulated as one or more of the following formulations selected from the group consisting of: a powder, a solution, a suspension, a topical preparation, an intravenous preparation, an oral preparation, an oral rinse, a nasal spray, an eye drop, a subconjunctival injection formulation, and an extended-release formulation, wherein optionally said extended-release formulation is inside the polymeric component of the contact lens.

2. A method for treating a patient afflicted with a virus infection by decreasing cell-to-cell viral infectivity transmission within tissues in said patient, the method comprising:
contacting tissues of said patient with a copolymer having bioadhesive and passivation components in an amount and for a time sufficient to limit the extent of viral transfection of neighboring uninfected cells and decreasing viral exposure of at-risk uninfected cells to reduce infection severity in said patient,
wherein said tissues of said patient have been transfected by said virus,
wherein said copolymer is a graft or block copolymer comprising either cationic, hydrophobic, or anionic moieties and a hydrophilic passivation moiety,
wherein optionally, the copolymer is formulated with a mucopenetrating agent and/or a macropinocytosis inhibitor, and
wherein preferably, the copolymer is formulated as one or more of the following formulations selected from the group consisting of: a powder, a solution, a suspension, a topical preparation, an intravenous preparation, an oral preparation, an oral rinse, a nasal spray, an eye drop, a subconjunctival injection formulation, and an extended-release formulation, wherein optionally said extended-release formulation is inside the polymeric component of the contact lens.

3. A method to reduce corneal nerve toxicity in a patient afflicted with cancer undergoing oncologic therapy comprising an antibody-drug conjugate, wherein said corneal nerve toxicity comprises damage to (an adverse effect) on the corneal nerve cells in said patient, the method comprising:
applying to said corneal nerve cells or tissues comprising said corneal nerve cells of said patient a formulation comprising a multifunctional graft copolymer in an amount and for a time effective to reduce corneal nerve toxicity in said patient,
wherein said reduction in corneal nerve toxicity in said patient is with respect to exposure by said corneal nerve cells or tissue comprising said corneal nerve cells to said antibody-drug conjugate in the absence of said copolymer,
wherein said formulation comprises said copolymer at a concentration ranging from 0.01% to 40%,
wherein the copolymer is selected from the group consisting of a cationic graft, cationic block, hydrophobic graft, hydrophobic block, anionic graft, and an anionic block copolymer and
wherein said copolymer displays electrostatic and steric mediating properties,
wherein optionally, the copolymer is formulated with a mucopenetrating agent and/or a macropinocytosis inhibitor, and
wherein preferably the copolymer is formulated as one or more of the following formulations selected from the group consisting of: a powder, a solution, a suspension, a topical preparation, an intravenous preparation, an oral preparation, an oral rinse, a nasal spray, an eye drop, a subconjunctival injection formulation, and an extended-release formulation, wherein optionally said extended-release formulation is inside the polymeric component of the contact lens.

4. A method to reduce corneal nerve toxicity in a patient at risk of such corneal nerve toxicity due to the imminent treatment with a therapeutic agent that can cause such toxicity, wherein said corneal nerve toxicity comprises damage to or an adverse effect on the corneal nerve cells in the subbasal plexus in said patient, the method comprising:
applying to said corneal nerve cells or tissues including the superficial cornea comprising said corneal nerve cells of said patient a formulation comprising a multifunctional graft copolymer in an amount and for a time effective to reduce corneal nerve toxicity in said patient,
wherein said reduction in corneal nerve toxicity in said patient is with respect to exposure by said corneal nerve cells or tissue comprising said corneal nerve cells to said therapeutic agent in the absence of said copolymer,
wherein said formulation comprises said copolymer at a concentration ranging from 0.01% to 40%,
wherein the copolymer is selected from the group consisting of a cationic graft, cationic block, hydrophobic graft, hydrophobic block, anionic graft, and an anionic block copolymer,
wherein said copolymer displays electrostatic and steric mediating properties, wherein optionally, the copolymer is formulated with a mucopenetrating agent and/or a macropinocytosis inhibitor, and
wherein preferably the copolymer is formulated as one or more of the following formulations selected from the group consisting of: a powder, a solution, a suspension, a topical preparation, an intravenous preparation, an oral preparation, an oral rinse, a nasal spray, an eye drop, a subconjunctival injection formulation and an extended-release formulation, wherein optionally said extended-release formulation is inside the polymeric component of the contact lens.

5. A method to treat or prevent a patient from having a sign and/or a symptom of one or more ocular adverse events associated with the chronic use of a topical ophthalmic drug product,
wherein the method comprises administering to said patient said topical ophthalmic drug product formulated with a multifunctional graft copolymer in an amount and for a time effective to treat or prevent said sign and/or symptom of said ocular adverse event associated with the chronic use of said topical ophthalmic drug product,
wherein said copolymer is formulated to be at a concentration ranging from 0.01% to 40%,
wherein the copolymer is a the multifunctional graft copolymer selected from the group consisting of a cationic graft, cationic block, hydrophobic graft, hydrophobic block, anionic graft, and an anionic block copolymer,
wherein said copolymer displays electrostatic and steric mediating properties, wherein preferably, said copolymer is formulated as an ophthalmic coformulation, optionally further comprising a mucopenetration enhancer and/or a macropinocytosis inhibitor, and
wherein preferably the copolymer is formulated as one or more of the following formulations selected from the group consisting of: a powder, a solution, a suspension, a topical preparation, an intravenous preparation, an oral preparation, an oral rinse, a nasal spray, an eye drop, a subconjunctival injection formulation, and an extended-release formulation, wherein optionally said extended-release formulation is inside the polymeric component of the contact lens.

6. A method to treat a patient displaying symptoms associated with a corneal toxicity common to symptoms associated with ADC therapy,
wherein the method comprises placing a multifunctional graft copolymer combined with contact lens placement on the eye in a manner selected from the group consisting of:
i. placing the multifunctional graft copolymer in the contact lens polymeric structure,
ii. placing the multifunctional graft copolymer in the water component of the contact lens,
iii. from the contact lens storage solution comprising the placing the multifunctional graft copolymer,
iv. from a drop on the contact lens, wherein said drop comprises the multifunctional graft copolymer,
V. from an implant,
vi. from application to the eye within 24 hours before a contact lens is placed, within 72 hours after a contact lens is placed,
wherein said placement treats said symptoms associated with said corneal toxicity in said patient,
where the copolymer is a the multifunctional graft copolymer selected from the group consisting of a cationic graft, cationic block, hydrophobic graft, hydrophobic block, anionic graft, and an anionic block copolymer,
wherein said copolymer displays electrostatic and steric mediating properties, and
wherein preferably the copolymer is formulated as one or more of the following formulations selected from the group consisting of: a powder, a solution, a suspension, a topical preparation, an intravenous preparation, an oral preparation, an oral rinse, a nasal spray, an eye drop, a subconjunctival injection formulation, and an extended-release formulation, wherein optionally said extended-release formulation is inside the polymeric component of the contact lens, and
wherein preferably the copolymer formulation is an ophthalmic coformulation, optionally further comprising a mucopenetration enhancer and/or a macropinocytosis inhibitor.

7. A method to treat a patient having a subbasal nerve plexus that is susceptible to adverse corneal nerve cell toxicity, wherein the method comprises:
concomitantly applying to the cornea of said patient a multifunctional graft copolymer and a neuroprotectant,
wherein concomitantly applying to the cornea of said patient said multifunctional graft copolymer and said neuroprotectant helps protect the subbasal nerve plexus of said patient from adverse corneal nerve cell toxicity,
wherein said copolymer is comprised in a formulation at a concentration ranging from 0.01% to 40%,
wherein the copolymer is a the multifunctional graft copolymer selected from the group consisting of a cationic graft, cationic block, hydrophobic graft, hydrophobic block, anionic graft, and an anionic block copolymer,
wherein said copolymer displays electrostatic and steric mediating properties, wherein preferably the copolymer is formulated as one or more of the following formulations selected from the group consisting of: a powder, a solution, a suspension, a topical preparation, an intravenous preparation, an oral preparation, an oral rinse, a nasal spray, an eye drop, a subconjunctival injection formulation, and an extended-release formulation, wherein optionally said extended-release formulation is inside the polymeric component of the contact lens, and
wherein preferably the copolymer formulation is an ophthalmic coformulation, optionally further comprising a mucopenetration enhancer and/or a macropinocytosis inhibitor.

8. The method of claim 1, wherein said patient is afflicted with cancer, wherein said cells not specifically targeted by said antibody are non-cancerous cells, and wherein said noncancerous cells of said patient do not express the epitope of said antibody-drug conjugate.

9. The copolymer for use according to claim 2, wherein the viral infection is selected from coronaviruses, influenzas viruses, Ebola viruses, and novel viruses transmitted through mucous membrane exposure, including, but not limited to the virus is SARS-COV-2.

10. The method of claim 1, wherein the copolymer is PLL-g-PEG.

11. The method of claim 2, wherein the copolymer is PLL-g-PEG.

12. The method of claim 3, wherein the copolymer is PLL-g-PEG.

13. The method of claim 4, wherein the copolymer is PLL-g-PEG.

14. The method of claim 5, wherein the copolymer is PLL-g-PEG.

15. The method of claim 6, wherein the copolymer is PLL-g-PEG.

16. The method of claim 7, wherein the copolymer is PLL-g-PEG.

17. The method of claim 1, wherein said conjugate comprises a toxic payload.

18. The method of claim 3, wherein said conjugate comprises a toxic payload.

19. The method of claim 6, wherein said conjugate comprises a toxic payload.

20. The method of claim 17, wherein said toxic payload is selected from the group consisting of a tubulin inhibitor, an anti-neoplastic agent and a DNA synthesis inhibitor.

21. The method of claim 18, wherein said toxic payload is selected from the group consisting of a tubulin inhibitor, an anti-neoplastic agent and a DNA synthesis inhibitor.

22. The method of claim 19, wherein said toxic payload is selected from the group consisting of a tubulin inhibitor, an anti-neoplastic agent and a DNA synthesis inhibitor.

23. The method of claim 2, wherein the virus is a virus transmitted through mucous membrane exposure.

24. The method of claim 4, wherein said therapeutic agent causing said toxicity is selected from the group consisting of: bortezomib, paclitaxel, and oxaliplatin.

25. The method of claim 5, wherein said chronic use of a topical ophthalmic drug product comprises a glaucoma medication.

26. The method of claim 7, wherein said neuroprotectant is a nerve growth factor.

27. The method of claim 1, wherein said patient undergoing therapy has cancer and wherein said antibody-drug conjugate targets cancer cells of said patient, and wherein said non-targeted cells are non-cancerous cells of said patient.

28. The method of claim 1, wherein said macropinocytosis inhibitor is selected from the group consisting of ethyl-isopropyl amiloride (EIPA), imipramine, phenoxybenzamine, vinblastine, flubendazole, terfenadine, a histamine H1-receptor antagonists, itraconazole an antifungal medicine, an α-adrenergic antagonist, auranofin, an anti-rheumatoid arthritis agent and a tricyclic antidepressant.

29. The method of claim 2, wherein said virus is selected from a coronavirus, an influenza virus, an Ebola virus.

30. The method of claim 2, wherein said virus binds to the ACE2 receptor, and wherein optionally, the virus is SARS-COV-2.


SEQUENCE LISTING (3 sequences)

SEQ ID NO: 1 - DNA, 25 bp, synthetic construct
  source 1..25: mol_type = other DNA
agaagattggttagatgatgatagt

SEQ ID NO: 2 - DNA, 25 bp, synthetic construct
  source 1..25: mol_type = other DNA
ttccatctctaattgaggttgaacc

SEQ ID NO: 3 - DNA, 24 bp, synthetic construct
  source 1..24: mol_type = other DNA
tcctcactgccgtcttgttgacca